SEQUENCE LISTING (23 sequences)

SEQ ID NO: 1 - DNA, 6859 bp, Homo sapiens
ccacccccttgccctctcccaggttctccttcaccccctgcagagccatcggctccctctatcctttgcaaccccctatgaatctttcctacttgtccaggtggagtgagtctgaggacagcagatgaacagacagaaactgaaagatccccaaaaaggtaagtggggagacattgaagatggggagtagcgggggtggacagagatcaagtaccctatctgaggagcaactgggtcctagcagttggtttggcctctggtattcccaaagctgcactggcacttgtgggggcccagcagtggtccaggctcacccctgagcttttggccttgctgcatcctgggacttctcctgctcccttcaatggctgggcagctcctagctactctgtgcctcttgcttccttaggattccctccatttccagcctacccttcagggtagctttgggaggcccaaaccatcaacgatgaaagaggaaaaggaaaggggaagagaacagaggcaaggtggtgcagtgggcagagggcaggcttctaggctctgaactcttccacccacatgctgcctggccttgggcaagtcatttgacttctgttagcctcggtttcacctcttcctttttaaatttaaattctgtttaaattgaagtgctactgcattcccgtggttggcagctcaaaaagcaagaaataaatcaggagaaaaagtctctcttctacctccatctcccaaccaaccagtttcccttttggatggcaaacaatattactagttttaaaatatatttttcaaagctagtttatgcatatctgagcaacatagataatccttcctttatacacattttattttatttatttttgagctagggtctcactctgttgcccaggctggagtgcagtagcacagtcatagatcactgcagcctcaaactcctgatcctctcacctcagcctctggagtagctgggactataggtgtgcaccaccatgcctgactaattttttaattttttgtagagatggggtcttgctatgttgcctaggctggtctcgaacttctggcctcaagtgatcctcctgccttggcctcccaaagtgctggaattagaggcatgaaccaccactcctggcctgccttatacacatcgtatacacaccattctacactttgctttttttcccccttaacagtggattatcactggagatcactgcatgtgtcaggacataaagcaattccccattggtttttttacagctatctggatgggactgcatttgtacagactctttacgatgtattcagccagtcttttactgatggttgtttaggttatgtcctagctttttgagctttggttttctcatctgtaaaacaggtaataataatgttgctttgtaggacagttgtgaagaattcaagagtgtgcatgtgaagtgctggaaccaagcactcaaagagtaggagtcagtattattgtccctcccatgtcacgaccctccatgggcccatagctagttcatcctccaagatgcctcttaaatgttgtctcctccaaaggctctccttttcttcctgcagcactcactactgcctgcatgactctgtgccaggtcgaaagatgcctggggacaaggtgtgcctcctccccatctttgtgcctcccagagtgtgtggcacagagaaggtgcccaggtgcacaggggagcttctgcctggagctgagcagaatgtgtgcttctctccgcaggatgagtgagagggtcgagcggaactggagcacgggcggctggctgctggcactgtgcctggcctggctgtggacccacctgaccttggctgccttgcagcctcccactgccacaggtttggaggaggtggcagggcccggggagggagtttcccaggacacctggggagctggacaccactccaccgtgttccatagcaggcaggtctcccaggcagagtaaaatccagagattctaggggttccggctcctcctctgctctttacccaggaatctctgcccaccccagctgtcccgccccagagaaggtctgggcagaccctttgctcacctctccttccaggctggaaggtgttctgtagtgagagaggccctcgggtgagggcttatctctagcctcctggcctaattctatggttttccttctgggatggctaaagcctgtgtaagcggatgatcctccccacgcattgtaagccctggggcccttgccctcaccctgaggaagctggggaggcggggctggtgcgggactctgtcttctgcccaggggccgtgctctgccaaagggtgaagagcacaggcctcacaggggagggaggaagaataggagctctgtgaagcggcgggaagatttcatccacttcctcctcccctgccactggcagtctcttccatagccctccctgcagcagggaggaggggctggaggccagcccgggtgggcgggagggagcggggtcgggcgtgttgggcgtctgatcctgggcggctcccctcagtgcttgtgcagcagggcacctgcgaggtgattgcggctcaccgctgctgcaaccggaaccgcatcgaggagcgctcccagacggtgaaatgctcctgtttttctggccaggtggccggcaccacgcgggcaaagccctcctgcgtggacggtgagtgagaggccaggacccgggcagggccctgagcagagccataaggaggcctgttcaggcctcgccatctgctggggatacaatttcatacccgtctcagggctttcatgagcagtctcaaatggtggggccccctctgggagaagagggcagacgtggccatccctgtgtcatagatgagaaaagctcttgttatgcgcacagcctcccacactcacgtgccctctgcctgttgcacacacaggagcgttccctcacacagccccactcctctccccacacacttcctccagctccttcccctgctgctccttcccctggctgtctcagcctactctcctctccttctcagcccttctctcacactttcctatgtggacacgtgtgatccttcacacccacactcattaccctaacactcatttggcaccccatacacatgcccactgtgtcacgcgcataccagctatgccagctatgcactcctggtctgtagacccctccagacgactcacacccttgctcacacaccacagagggagtatactggttgtgcatcgatgctagtgacatggtcaccttagagacatttttgcctgccttcacccgtcactgtctggggagatgcagggatcggtgggattaaagttgagagggttacgtccccaggcctcaggctgagtactgcctctctgggcagcctggagtaggggaaagacaagggaaatgcatggctcaaggccaggggtgtgtggaaacgtgcacctcattcgcagacctgctcttggctgcccactgtgctcgtagagaccctagagctgcactccgcctcctgctcccacagcctccatcgtcctgcagagatggtggtgtcagatggagccctgcctgccgggggaggagtgtaaggtgctcccggacctgtcgggatggagctgcagcagtggacacaaagtcaaaaccaccaaggtaccctgggtgggcacctcatcccaccctccccttccctctctaccaagggcagaaaagggggcacaggagccttggaggtctaggatgtcagtgtcccaaggtgagatcgtgaaccatgaaattcacagacagtcctctcaggtctgttctcactggggactggagttaggggggtcaggacttgaaatccagtgtgtcctgggaagattcttgatgtccatatacgctggtttcctcctgtctgcctggaattagtttgcctccagaaaatatcaggcctataaaaacatagtccatggaatactatgcagccataaaaaatgatgagttcatgtcctttgtagggacatggatgaaattggaaatcatcattctcagtaaactatcgcaagaacaaaaaaccaaacaccgcatattctcactcataggtgggaactgaacaatgagatcacatggacacaggaaggggaatatcacactctggggactgtggtggggtggggggaggggggaggggtagcattgggagatatacctaatgctagatgacgagttagtgggtgcagcgcaccagcatggcacatgtatacatatgtaactaacctgcacaatgtgcacatgtaccctaaaacgtaaagtataatttaaaaaaaaaaaaacaacatattagagcagcaaaaaaaaaaaaaaaaaaaaaaaagagcttaaaacagaactaccattcaacccagccatcctattactggtatatacccaaaggaaaataaatcattttgccaaaaagacacatgcacttgtatgttcatcaaagcagtattcataatagcaagggcatataatcagtctaggtgccaatcaacattgaataggataaagaaaatgtggcacttatataccatagaatactacacagccataaaaaaaataatgttttctgtaacaccctggttgcagctgaaggccatgatcctaagggacttagcatggcaacggaaaatcaaataccacatgttctcacttctgagagctaaacattgggtacacatggacataaagatgggaacagtggacgatggtgactaccagagcagtgagagagggaggggagcaagggctgaaaaactacctattgggtcctatgcttactacatgggtaatgagatcaatggcactccaaacctcagtgtcatgcaatatactcatgtaacaaacctgcacatgtaatccctgaatctaaaataaaagttgaaatattaaaaaaaaaaaaaaaaaaaacatagtcgagaatgggaaaaattataagataattgagggaggaattgctacttctacctgagctccaggtacagtagtcactctgtcactcacctctgccgtgaccatcccccacccccaccccattcccactgttgttgactccctacagttcatacctaccctcctctagtcagatttctctgtccactgtcacagcacccaagaggattattattttcatggtttgctgcctgctccacagaacaaggggtaattaaattttttatcaaactcgttagcagctccatgtgtgtcatggctaatcagccgcattgctctgccgtcaccattggtacagaagagtgacactgcagggcaagccagggttctgcctttggctttccttccctcccaccacacacagcaaacaagcaaacacataagaaaatagagaaggagggctgggtgcggtggctcatgcctgtaatcccagcactttgggagacagaggtgggtgaatcaagaggtcaggagttcaagaccaacctggtcaacatggtgaaaacccgtctctactaaaaatacaaaaattagctgggcataatggcacacgcctgtaatcccagctacttgggaggctgaggcaggagaattgcttgaacctaggaggcagaagttgcggtaagcagagatcatgccactgcactccagtctgggcgacagagtgagactccatctcaaaaaaagaaaaaaagaaaaaaaaatagagaaggaaaggcagcctttggcttctgaagagagcaaccattttcacagagcacacaacaaaaggagctacagcaagaaaaagtttggctctaaagtagagcatcctctctctatccggaggcagggatgagggacagccattctgagtatatttaagatcagaagagaatgagacccactacagagcttgtgacatttgtcaccatgttataggtttgggtttgtggcctgtaaggaagtgcttgcaatttgggtagctactctgaagcaaacagtggggaggcttgtctgacgtatgtttgcatgggtcaccaggccatgctttcccattccggcctgctttagggtgagaggattggcagcaatgtcctctaagtgacctgcatattctgggtagtctctggcatgctggaatggaataggcattcccttctctaaccttaccctctcgcttcttcaccaggtcacacgatagctcttgggggtcacggcctggacaagaaaggcttgactgagccgtgaactgaagaatggtatccagtcatcagccaggaaagatggggattcacttacatgcctcatgtcaaatgcagcatcagtctttccggggcatttcagttaagctgctcagcagatatggatggatctgcaatcacatacctaatgtggagctgggcttttctggagacacgaaggtcaacacacaattcctgcccttaaggaatgtccagttgaattggagagttgatgacagacaatttagataatttaggttaaagtacttgataccagactgcggcttctgggccacatgctatggcatgatgggggtttgggaatgagattcccacagttcttcagataccctgtggccacagggcatagaaacaagaggtcacattcagcacccaccacctccctctttcgcatcagttctgaatccccagcaagctgttaacatgttgcaggaaaacactctccccttatgccagaccagcagtatctcatttggatgggattggattgacttgcggaaggaaagtaaaaataaagccaaataacttcccaaatctttggcacaggtatgggccattcattgggtatgggatggacaggactttaaaatcatctccatccaagcacctcaggtttttgtttgtttgcttattt

SEQ ID NO: 2 - DNA, 1077 bp, Homo sapiens
ggtggaattcgtggagtgagtctgaggacagcagatgaacagacagaaactgaaagatccccaaaaaggatgagtgagagggtcgagcggaactggagcacgggcggctggctgctggcactgtgcctggcctggctgtggacccacctgaccttggctgccctgcagcctcccactgccacagtgcttgtgcagcagggcacctgcgaggtgattgcggctcaccgctgctgcaaccggaaccgcatcgaggagcgctcccagacggtgaaatgctcctgtttttctggccaggtggccggcaccacgcgggcaaagccctcctgcgtggacgcctccatcgtcctgcagagatggtggtgtcagatggagccctgcctgccgggggaggagtgtaaggtgctcccggacctgtcgggatggagctgcagcagtggacacaaagtcaaaaccaccaaggtcacacgatagctcttgggggtcacggcctggacaagaaaggcttgactgagccgtgaactgaagaatggtatccagtcatcagccaggaaagatggggattcacttacatgcctcatgtcaaatgcagcatcagtctttccggggcatttcagttaagctgctcagcagatatggatggatctgcaatcacatacctaatgtggagctgggcttttctggagacacgaaggtcaacacacaattcctgcccttaaggaatgtccagttgaattggagagttgatgacagacaatttagataatttaggttaaagtacttgataccagactgcggcttctgggccacatgctatggcatgatgggggtttgggaatgagattcccacagttcttcagataccctgtggccacagggcatagaaacaagaggtcacattcagcacccaccacctccctctttcgcatcagttctgaatccccagcaagctgttaacatgttgcaggaaaacactctccccttatgccagaccagcagtatctcatttggatgggattggattgacttgcggaaggaaagtaaaaataaagccaaataacttcccaaaaaaaaaaaaaa

SEQ ID NO: 3 - protein, 133 aa, Homo sapiens
MSERVERNWSTGGWLLALCLAWLWTHLTLAALQPPTATVLVQQGTCEVIAAHRCCNRNRIEERSQTVKCSCFSGQVAGTTRAKPSCVDASIVLQRWWCQMEPCLPGEECKVLPDLSGWSCSSGHKVKTTKVTR

SEQ ID NO: 4 - protein, 103 aa, Homo sapiens
ALQPPTATVLVQQGTCEVIAAHRCCNRNRIEERSQTVKCSCFSGQVAGTTRAKPSCVDASIVLQRWWCQMEPCLPGEECKVLPDLSGWSCSSGHKVKTTKVTR

SEQ ID NO: 5 - protein, 75 aa, Homo sapiens
CEVIAAHRCCNRNRIEERSQTVKCSCFSGQVAGTTRAKPSCVDASIVLQRWWCQMEPCLPGEECKVLPDLSGWSC

SEQ ID NO: 6 - protein, 87 aa, Homo sapiens
GTCEVIAAHRCCNRNRIEERSQTVKCSCFSGQVAGTTRAKPSCVDASIVLQRWWCQMEPCLPGEECKVLPDLSGWSCSSGHKVKTTK

SEQ ID NO: 7 - DNA, 5745 bp, Mus musculus
aatggctgcctttgccttgggacagcctaagctggagttcctcatcctccctttctcttactctcttccaagcttcctcttagcccctccaggggtctggtctctctccattctgaacaaccgccatattgttcatcagatggaacaagtctgagaccagcagatgaacaggcagaggcctgaagaccccaggaacagtaagtgagacatctggagatgagaattgaggagaagactgcccctccaagggagcagctgtttccttctttttggcccctggtattcctagaactgtgctgtcaattgctggggacagtgaccaggctaacccgtggcctcctggggctcttcctgtccccttctgtggctagtttctctcagcttctttttgttgctacttttcgggtttggcctctggttcttacccacttctggtacagatgttggaggtcttgcttggttcatagaagacaggaggaagagaaagcctcaggcaggcttctgggtcctgacccatgaccctggcccttgggcaaagctttcaacttctaatgggggaggaggtggtcttccttccttccttccttccttccttccttccttccttccttccttccttctttccttccttctttccttccttccttccttccttccttccttccttccttctttccttccttccttctttccttccttccttccttccttctttccttccttccttccttcctttcttccttccttccttctttccttccttccatttatttatttatttatttatttattcattcattcattcattcattcattcattcattcattcattctttttcacgtgtgtgcatgatgtgtgtgcatgtctgcatccacatgaaagccagaggaggcttatttgatagagacatcctgctctgtcacttgctaccttgctccgccttggtgagggtctttcactgactctagctctaggctgtccagcaatccagcaatcttcttacgtagtctagctttttatatctgtgatggtgttcccacgcttgcccttactcactgagccatccccctcagctctgtgctttgtgtttgtttgtttgtttgtttgtttgtttgtttgtttgagacagggtcttcctatagagtcctagctagacttggaactcactatgtagatcaggctgtcctcaaactttcaatgatccgtctgctgttgcttctagagtgctaggaatacgtttgccataataacaagccttcattcctttctgttaaaaatttaaattgtgtttagactgaattgtactgctactacagtcccgtgatttgaaacttaaacacgtaaaagaaagacattgggggaaaataactcatcttcttacaacttcttttttgtttttgtttttgtttttcgagacagggtttctctatatagccctggctgtcctggaactcactttgtagaccaggctggcctcgaactcagaaatcctcctgcctctgcctcccaaatgctggggttaaaggtgtgcgccaccacgcccggcttacttacaacttctagccaagcaaaaagtttcctttctcaatggcaaacagagcaggtatgtacatattaagcaacacacatacacactctctccctcatacacacttgttgcttttttcccatcctttacaggccgactggtttctcagtaaagtctctgtttgtgtcattacaaaattcctcgatggctttggttttctctgtgttcccagcagccatctggagggaactgcattttcaagttctccatggtgtactcagcccctccctctactgatggctggtcaggttgtatccagacttttggagctttatttacaaacgatcatagcaatctctcgtgtagcgagatatcaccaagagcacacgagtgaagtgttccactcaagagctcaaagagtgggagtcggcatcattgtccagtccatggcacagcccttaatttactctgcgagatctctgaaatgtctcctccacaggctctgccttcctgttccttgatccctctctgtcaatcatcatatcctacagtgacatcccgccccaccgaaagctgcctggagacaaggtctgtatcttgcctaaccttgggcttctagcgcatggcatagagaaggggctgagtgcaggagcagagggagtgtctgcttcgagctgagtagggtgtatacttctctccacagaccgatggagaggcccaccagcaactggagcgcaggcagctgggtgcttgcactgtgcctcgcctggctgtggacgtgtccggcctctgcttccttgcagcctccaacatccgcaggtctggggaaggtggtgagacgagggaaggggtttttacaggatgcttggagagcaagaaggcactccagcccatcccacaaagcaggcagatctctggggcagagtaaaatctgcagatgtgactcctccagtcattacccaggaaactgtcctcttcccgtccccagtctagagaaggcctgggaagacccattggcaccatctcttccaaggggaaggtgttctgcagtgaggccctcgggcggaggcttaagtctagtctcctggcctaattctggggttttccttctggaatggctaaagcctctgtaagcggatgatcctcccccacgcattgtaagctctggggcccttgccctcaccctgagaaagtggaaggtgggggctggtgtgggactccagctttcaaagagtcatgaacagtgtgtgttgaaggtctgatcccagccttcacctatatgctagaaaacactgctgctgagccgtagtcccagcgctgggtttctcttaaatgacttttccatgagcaaggaggagggggttgagggcctgcagcagggtaggagagagggaggagggcagggttggagacctgggtctcctgagcttgtaattgctggctgcatcagtcctagtgaagcagggcacctgcgaggtgattgctgcacatcgctgctgcaaccggaaccgcattgaggagcgctcccagacggtcaaatgctcctgcctgtccggccaggtggctggcaccaccagagcaaagccctcctgcgtggacggtgagtgagaggccaggccctgggcagagcagagataagaaagtccacttaggcctcaccatctgctgagggtctagtttcatagctatcttaaggctgaccttatctgtctcaagtggagaagagggcagacatggcccgccctgtgtcatagaggaagaaagctcttgttacagaccatgtacacacctgccacctggcccattccctcaagccagctcctctggctaaacacttctcgagggaggtcctcttccacacgtctcttattctcaacctgctctgtgaggaacatgggatccctgatacagcacacccactctccttcctttctgtgcacactgtgttctcacgctctggtggcatccagagcgtcaccatacctgtgctcccatgccagcctgtgcccgtctggatgtggcttggatactagctcatataccctaaagagggcactgaagatggtacctctgtgcgaacgacagctgcccctagcttgatgccctcctcccgagttggtgtgatgcagtgatgaagtttcctgggatagaccccagtccacagcctgtgtacagtgctgcctccttggacagcccagagtagggggatcccagggggaaggtggtaatcagcgcctgcagttggtgggaaggaagctacacctcattcacagaccagctatgaccggtgctgtgctcccagaccttagagccacacctgcctcctgccccacagcctccatcgtcctgcagaagtggtggtgtcagatggagccctgcctgctgggagaggagtgtaaggtgctcccggacctgtcagggtggagctgcagcagtggacacaaggtcaaaaccaccaaggtattgagacgggtggctcaccctcccctcctgctcctccctcacccatgatagcacaggggccaaaccctggagggccaggccctgctactggtgatggcatgcctgccttgactttctcctcttcgattcccatgggtttacccctagaaaatatcaggccttttaaaacaaaatggagcagggcacagaggcacacatctgtaatcctagcactcaggaacaagatcaggagttcagttagcctgggttgcatgatacctgtatccaaaaatacaaaaacaaacaaacaaaaacaaaacaaaacaggaaaaaaaaaaccctaaggaaaaaaaatgtagagaactgagaaaattacctagatcaaccagggatgaatttatccttctatctgagttccagaaactgtatcctctctgcctgccacctcaccacagactgtcccctgtcacctacaccactcctaccccttcccctcccacttcctgcctgccctcccctagtcggatgctctgtcctcagtaacagcacctaagaagattattatcgtcacagcttgccgcctgccccacaaaacaaggggtaattaaaattttatcaaactctttagcggttccacaagtgccgtggctaattagttgtatgtcattgccaccatcacctgtgcaagtgagtgacatttccaggcaagccaaggcagtgtccccgccaccagccccactacacacagcaagcaggttaactaacagaagcaaaaccacttctggggttctgaggaaaatggaggttttcacagaacacacgacactgggaacttcagcagaaaaaggtttgggggctggagaggtagctcagtggttaggagcactttttgcttttacagaagacccaggttcaatttccagcacctccagtagcttagtactctctgtaacacagtcctaaaggatccaataccctcttctggcctccttgggcaccacacatgtggtggacagatgttcatgcagacacagttctcatatacataaaacttaaaatgtggggagggagcagttggctgactaaaaggcagggcagcccatctgtaggcagtaaaggggggcagccattctgagaatgtttattaagacttgcaatgtgagacctatcacagagccagggacatttggcatactgttacaggcctgaaagacagatcactgcctttgcaatccgtgtagtgactcagaagcaaacggcagtgaggtttgcctgacgggtgccagcatggatggatcaccaggccacaggtgctcagcttgggtggtgttaggagactggcagcggtgacctctcggtgactcccctatgctggggctctctggcatgccacaatggccggttccctgttgctttctcaagccttcccctacctgacgatttgctttctaggtcacacggtaactctcggaggtcatggcttaggtaggacagccttgactgagctcgggactgaagaaaggcctggtcaccagacagcagataagaggacttacctggacatgtgcccatgtcaagtgtaacatagaccggccagggccctgggcagcactctgttcagctaaaatgcttggatctttggccacacacttgagagacctgtgctctcctatgaacaaagtcaacacacaaacctatccttaaggaatatcttgtcaagttaaggagtggatgacaggcaatttcaacagtttaaagtgtttggtaccgtggc

SEQ ID NO: 8 - DNA, 801 bp, Mus musculus
tctctctccattctgaacaaccgccatattgttcatcagacggaacaagtctgagaccagcagatgaacaggcagaggcctgaagaccccaggaacagctctgccttccttgttccttgatccctctctgtcaatcatcatatcctacagtgacatcccgccccaccgaaagctgcctggagacaagaccgatggagaggcccaccagcaactggagcgcaggcagctgggtgcttgcactgtgcctcgcctggctgtggacgtgtccggcctctgcttccttgcagcctccaacatccgcagtcctagtgaagcagggcacctgcgaggtgattgctgcacatcgctgctgcaaccggaaccgcattgaggagcgctcccagacggtcaaatgctcctgcctgtccggccaggtggctggcaccaccagagcaaagccctcctgcgtggacgcctccatcgtcctgcagaagtggtggtgtcagatggagccctgcctgctgggagaggagtgtaaggtgctcccggacctgtcagggtggagctgcagcagtggacacaaggtcaaaaccaccaaggtcacacggtaactctcggaggtcatggcttaggtaggacagccttgactgagctcgggactgaagaaaggcctggtcaccagacagcagataagaggacttacctggacatgtgcccatgtcaagtgtaacatagaccggccagggcccctgggcagcactctgttcagctaaaatgcttggatctttggccacacacttgagagacctgtgctctcctatg

SEQ ID NO: 9 - protein, 132 aa, Mus musculus
MERPTSNWSAGSWVLALCLAWLWTCPASASLQPPTSAVLVKQGTCEVIAAHRCCNRNRIEERSQTVKCSCLSGQVAGTTRAKPSCVDASIVLQKWWCQMEPCLLGEECKVLPDLSGWSCSSGHKVKTTKVTR

SEQ ID NO: 10 - protein, 103 aa, Mus musculus
SLQPPTSAVLVKQGTCEVIAAHRCCNRNRIEERSQTVKCSCLSGQVAGTTRAKPSCVDASIVLQKWWCQMEPCLLGEECKVLPDLSGWSCSSGHKVKTTKVTR

SEQ ID NO: 11 - protein, 75 aa, Mus musculus
CEVIAAHRCCNRNRIEERSQTVKCSCLSGQVAGTTRAKPSCVDASIVLQKWWCQMEPCLLGEECKVLPDLSGWSC

SEQ ID NO: 12 - protein, 87 aa, Mus musculus
GTCEVIAAHRCCNRNRIEERSQTVKCSCLSGQVAGTTRAKPSCVDASIVLQKWWCQMEPCLLGEECKVLPDLSGWSCSSGHKVKTTK

SEQ ID NO: 13 - DNA, 6859 bp, Homo sapiens; identical to SEQ ID NO: 1

SEQ ID NO: 14 - DNA, 1144 bp, Homo sapiens
ggtggaattcgtggagtgagtctgaggacagcagatgaacagacagaaactgaaagatccccaaaaaggatgagtgagagggtcgagcggaactggagcacgggcggctggctgctggcactgtgcctggcctggctgtggacccacctgaccttggctgccctgcagcctcccactgccacagtgcttgtgcagcagggcacctgcgaggtgattgcggctcaccgctgctgcaaccggaaccgcatcgaggagcgctcccagacggtgaaatgctcctgtttttctggccaggtggccggcaccacgcgggcaaagccctcctgcgtggacgacctgctcttggctgcccactgtgctcgtagagaccctagagctgcactccgcctcctgctcccacagcctccatcgtcctgcagagatggtggtgtcagatggagccctgcctgccgggggaggagtgtaaggtgctcccggacctgtcgggatggagctgcagcagtggacacaaagtcaaaaccaccaaggtcacacgatagctcttgggggtcacggcctggacaagaaaggcttgactgagccgtgaactgaagaatggtatccagtcatcagccaggaaagatggggattcacttacatgcctcatgtcaaatgcagcatcagtctttccggggcatttcagttaagctgctcagcagatatggatggatctgcaatcacatacctaatgtggagctgggcttttctggagacacgaaggtcaacacacaattcctgcccttaaggaatgtccagttgaattggagagttgatgacagacaatttagataatttaggttaaagtacttgataccagactgcggcttctgggccacatgctatggcatgatgggggtttgggaatgagattcccacagttcttcagataccctgtggccacagggcatagaaacaagaggtcacattcagcacccaccacctccctctttcgcatcagttctgaatccccagcaagctgttaacatgttgcaggaaaacactctccccttatgccagaccagcagtatctcatttggatgggattggattgacttgcggaaggaaagtaaaaataaagccaaataacttcccaaaaaaaaaaaaa

SEQ ID NO: 15 - protein, 169 aa, Homo sapiens
MSERVERNWSTGGWLLALCLAWLWTHLTLAALQPPTATVLVQQGTCEVIAAHRCCNRNRIEERSQTVKCSCFSGQVAGTTRAKPSCVDDLLLAAHCARRDPRAALRLLLPQPPSSCRDGGVRWSPACRGRSVRCSRTCRDGAAAVDTKSKPPRSHDSSWGSRPGQERLD

SEQ ID NO: 16 - protein, 139 aa, Homo sapiens
ALQPPTATVLVQQGTCEVIAAHRCCNRNRIEERSQTVKCSCFSGQVAGTTRAKPSCVDDLLLAAHCARRDPRAALRLLLPQPPSSCRDGGVRWSPACRGRSVRCSRTCRDGAAAVDTKSKPPRSHDSSWGSRPGQERLD

SEQ ID NO: 17 - DNA, 815 bp, Homo sapiens
gacatcagaaccccaggctctccagcctttggacttcaggactgacacaagcaacctgctgggttcttaggcctttggcttggtactgagacttacaccatcagcttccctggtcctgagacttttggacttggattgagccacgctactggcatcccaggatctccagcttgcagacagcctgtcgtgggacttcacagcctccataattatagaatggcaatggtctctgcgatgtcctgggtcctgtatttgtggataagtgcttgtgcaatgctactctgccatggatcccttcagcacactttccagcagcatcacctgcacagaccagaaggagggacgtgtgaagtgatagcagcacaccgatgttgtaacaagaatcgcattgaggagcggtcacaaacagtaaagtgttcctgtctacctggaaaagtggctggaacaacaagaaaccggccttcttgcgtcgatgcctccatagtgattgggaaatggtggtgtgagatggagccttgcctagaaggagaagaatgtaagacactccctgacaattctggatggatgtgcgcaacaggcaacaaaattaagaccacgagaattcacccaagaacctaacagaagcatttgtggtagtaaaggaaaaccaaccctctggaaaatacattttgagaatctcaaacatctcacatatatacaagccaaatggatttcttacttgcactttgactggctaccagataatcacagtgcgtttactgtgtgtaacgaaatatcctacagtgagaagacacagcgttttggcaacaccatgg

SEQ ID NO: 18 - protein, 133 aa, Homo sapiens [sequence omitted]

SEQ ID NO: 19 - protein, 108 aa, Homo sapiens
SLQHTFQQHHLHRPEGGTCEVIAAHRCCNKNRIEERSQTVKCSCLPGKVAGTTRNRPSCVDASIVIGKWWCEMEPCLEGEECKTLPDNSGWMCATGNKIKTTRIHPRT

SEQ ID NO: 20 - protein, 75 aa, Homo sapiens
CEVIAAHRCCNKNRIEERSQTVKCSCLPGKVAGTTRNRPSCVDASIVIGKWWCEMEPCLEGEECKTLPDNSGWMC

SEQ ID NO: 21 - protein, 87 aa, Homo sapiens
GTCEVIAAHRCCNKNRIEERSQTVKCSCLPGKVAGTTRNRPSCVDASIVIGKWWCEMEPCLEGEECKTLPDNSGWMCATGNKIKTTR

SEQ ID NO: 22 - DNA, 801 bp, Mus musculus
tacagtgaacgagcccaacgtcttcacaactgtggaaacgggactggaagagaaaagcttgcctttttttcttttcttttcttttcttttttttttttcaattcttaataaagaattgctgggaagcattctctttgaaaaatctcagaactgtggcacagatggattttaaaaagtgttagctctttccaatgagcactaggagggttccctgctcttggctggatttttcagagaatggcaatggtctctgcaatgtcctgggccctgtacttgtggataagtgcttgtgcgatgctgctctgccatgggtcactccaacacaccttccagcagcatcacctgcaccggccagaaggagggacctgtgaagtgatcgcggcccacaggtgttgtaacaagaaccgcatcgaggagcggtcacaaacagtgaagtgttcctgtttacctgggaaagtggctgggacaacaagaaaccgaccttcctgtgtggatgcctccatagtaattgggaaatggtggtgtgagatggagccctgcctagaaggagaagaatgtaagacactccctgacaattctggatggatgtgtgctacaggcaacaagattaagactacacgaattcacccaagaacctaacagaagcatttgttatataaataggaaaaagaacaacctgtggaatatacgttgtgaggatttaaaacatcttccatagttgcaagccaagtggatctcttatctgcactttggttaccagataaccacagtgcacttactctgatacacagtatcccaaaa

SEQ ID NO: 23 - protein, 133 aa, Mus musculus
MAMVSAMSWALYLWISACAMLLCHGSLQHTFQQHHLHRPEGGTCEVIAAHRCCNKNRIEERSQTVKCSCLPGKVAGTTRNRPSCVDASIVIGKWWCEMEPCLEGEECKTLPDNSGWMCATGNKIKTTRIHPRT

(19) United States
(12) Patent Application Publication
(10) Pub. No.: US 2009/0087474 A1
(43) Pub. Date: Apr. 2, 2009

(54) Therapeutic use of growth factors,nsg29 and nsg31

(76) Inventors: Thomas N. Petersen, Kobenhavn (DK); Nikolaj Blom, Copenhagen (DK); Mette Gronborg, Copenhagen (DK); Philip Kusk, Lynge (DK); Soren Brunak, Hellerup (DK); Teit E. Johansen, Horsholm (DK); Lars U. Wahlberg, Asnaes (DK)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 11/794,993

(22) PCT Filed: Jan. 9, 2006
(86) PCT No.: PCT/EP06/00178

§ 371 Date: Nov. 18, 2008

(51) Int. Cl. A61K 48/00; C07K 14/47; C12N 5/10; C12N 15/12; C12N 15/70; C12N 15/74; A61K 35/12; A61K 31/711; A61P 25/28; C12N 5/00; C12N 5/02; A61P 29/00; A61P 15/16; A61P 15/10; A61P 35/00; A61P 15/00; A61P 25/02; A61P 25/00; C07K 16/18; A61P 25/16; A61K 38/17; C12N 15/85; C12N 1/15; C12N 1/19; C12N 1/21; A61L 27/54

(52) U.S. Cl. 424/425; 530/350; 530/324; 536/23.5; 435/320.1; 435/252.33; 435/254.2; 435/254.11; 435/348; 435/352; 424/424; 424/93.2; 424/93.21; 514/12; 514/44; 435/377; 435/375; 530/387.9; 530/387.3

(57) ABSTRACT

The present invention relates to the field of therapeutic use of proteins, genes and cells, in particular to the therapy based on secreted therapeutic proteins, NsG29 and NsG31. NsG29 and Ns31 are members of a newly identified family of growth factors with a specific cystein pattern and characterised by expression in the nervous system. The secreted growth factors have potential for the treatment of disorders of the nervous system. The invention also relates to bioactive NsG29 and NsG31 polypeptide fragments and the corresponding encoding DNA sequences.

### CROSS REFERENCE TO RELATED APPLICATIONS

This application claims priority from Danish Application No. PA 2005 00035, filed Jan. 7, 2005, Danish Application No. PA 2005 01096, filed Aug. 1, 2005, Danish Application No. PA 2005 00037, filed Jan. 7, 2005, and Danish Application No. PA 2005 01097, filed Aug. 1, 2005. The entire content of each of these prior applications is incorporated herein by reference in its entirety.

### TECHNICAL FIELD

The present invention relates to the field of therapeutic use of proteins, genes and cells, in particular to the therapy based on the biological function of secreted therapeutic proteins, NsG29 and NsG31, in particular for the treatment of disorders of the nervous system. The invention also relates to bioactive NsG29 and NsG31 polypeptide fragments and the corresponding encoding DNA sequences.

### BACKGROUND ART

Extracellular proteins play important roles in, among other things, the formation, differentiation and maintenance of multicellular organisms. The fate of many individual cells, e.g., growth, proliferation, migration, differentiation, or interaction with other cells, is typically governed by information received from other cells and/or the immediate environment. This information is often transmitted by secreted polypeptides (for instance, mitogenic factors, survival factors, cytotoxic factors, differentiation factors, neuropeptides, and hormones) which are, in turn, received and interpreted by diverse cell receptors or membrane-bound proteins. These secreted polypeptides or signaling molecules normally pass through the cellular secretory pathway to reach their site of action in the extracellular environment.

Disorders such as Parkinson's disease, Alzheimer's disease, Huntington's disease, multiple and amyotrophic lateral sclerosis, stroke, schizophrenia, epilepsy and peripheral neuropathy and associated pain affect millions of people. It is the loss of normal neuronal function, which produces the behavioral and physical deficits which are characteristic of each of the different neurological disorders. In addition to chronic and acute neurodegenerative disorders, the aging process, physical trauma to the nervous system, and metabolic disorders may result in the loss, dysfunction, or degeneration of neural cells accompanied by the associated behavioral and physical deficits. Many of these diseases are today incurable, highly debilitating, and traditional drug therapies often fail. There is thus a great medical need for new therapeutic proteins that are disease modifying and not only for symptomatic use.

Several secreted factors with expression in the nervous system or associated target areas have important therapeutic uses in various neurological indications associated with reduction or loss of neuronal functions. E.g. NGF is a candidate for treatment of Alzheimer's disease, Neublastin (Artemin) a candidate for treatment of peripheral neuropathy, and GDNF is a candidate for treatment of Parkinson's Disease.

### SUMMARY OF THE INVENTION

The present invention relates to various aspects and uses of Nerve Survival and Growth factor 29 and 31, NsG29 and NsG31.

In a first aspect the invention relates to an isolated polypeptide for medical use, said polypeptide comprising an amino acid sequence selected from the group consisting of:

a) the amino acid sequence selected from the group consisting of SEQ ID No. 3, 4, 5, 6, 9, 10, 11, 12, 15, 16, 18, 19, 20, 21, and 23;
b) a sequence variant of the amino acid sequence selected from the group consisting of SEQ ID No. 3, 4, 5, 6, 9, 10, 11, 12, 15, 16, 18, 19, 20, 21, and 23, wherein the variant has at least 70% sequence identity to said SEQ ID No.; and
c) a biologically active fragment of at least 50 contiguous amino acids of any of a) through b).

In a further aspect the invention relates to an isolated nucleic acid molecule for medical use comprising a nucleotide sequence selected from the group consisting of:

a) a nucleotide sequence coding for a polypeptide having the amino acid sequence selected from the group consisting of SEQ ID No. 3, 4, 5, 6, 9, 10, 11, 12, 15, 16, 18, 19, 20, 21, and 23;
b) a nucleotide sequence coding for a sequence variant of the amino acid sequence selected from the group consisting of SEQ ID No. 3, 4, 5, 6, 9, 10, 11, 12, 15, 16, 18, 19, 20, 21, and 23, wherein the variant has at least 70% sequence identity to said SEQ ID No.;
c) a nucleotide sequence coding for a biologically active fragment of at least 50 contiguous amino acids of any of a) through b);
d) a nucleotide sequence selected from the group consisting of SEQ ID No. 2, 8, 14, 17, and 22;
e) a nucleotide sequence having at least 70% sequence identity to a coding sequence selected from the group consisting of the coding sequence of SEQ ID No. 2, 8, 14, 17, and 22;
f) a nucleic acid sequence of at least 150 contiguous nucleotides of a coding sequence selected from the group consisting of the coding sequence of SEQ ID No. 2, 8, 14, 17, and 22;
g) the complement of a nucleic acid capable of hybridising with a nucleic acid molecule having the sequence of the coding sequence of SEQ ID No.: 2, 8, 14, 17, and 22 under conditions of high stringency; and
h) the nucleic acid sequence of the complement of any of the above.

In a further aspect the invention relates to a vector comprising the nucleic acid molecule of the invention.

In a further aspect, the invention relates to an isolated host cell transfected or transduced with the vector of the invention.

In a further aspect, the invention relates to a packaging cell line capable of producing an infective virus particle, said virus particle comprising a Retroviridae derived genome comprising a 5′ retroviral LTR, a tRNA binding site, a packaging signal, a promoter operably linked to a polynucleotide sequence encoding the polypeptide of the invention, an origin of second strand DNA synthesis, and a 3′ retroviral LTR.

In a further aspect, the invention relates to an implantable biocompatible cell device, the device comprising:

i) a semipermeable membrane permitting the diffusion of a protein of the invention and/or a virus vector; and
ii) a composition of cells according to the invention or a packaging cell line according to the invention.

In a further aspect, the invention relates to a pharmaceutical composition comprising

i) the polypeptide of the invention; or
ii) the Isolated nucleic acid sequence of the invention; or
iii) the expression vector of the invention; or
iv) a composition of host cells according to the invention; or
v) a packaging cell line according to the invention; or
vi) an implantable biocompatible cell device according to the invention; and
vii) a pharmaceutically acceptable carrier.

In a further aspect, the invention relates to the use of

i) the polypeptide of the Invention; or
ii) the Isolated nucleic acid sequence of the Invention; or
iii) the expression vector of the invention; or
iv) a composition of host cells according to the invention;
v) an implantable biocompatible cell device according to the Invention; or
vi) a packaging cell line according to the invention;
for the manufacture of a medicament.

In a further aspect, the invention relates to a method of treatment of a pathological condition in a subject comprising administering to an individual in need thereof a therapeutically effective amount of:

i) the polypeptide of the invention; or
ii) the isolated nucleic acid sequence of the invention; or
iii) the expression vector of the invention; or
iv) a composition of host cells according to the invention; or
v) an implantable biocompatible cell device according to the invention; or
vi) a packaging cell line according to the invention.

In a further aspect, the invention relates to the use of

i) the polypeptide of the invention; or
ii) the Isolated nucleic acid sequence of the invention; or
iii) the expression vector of the invention; or
iv) a composition of host cells according to the invention;
v) an implantable biocompatible cell device according to the invention;
as a male contraceptive.

In a further aspect, the invention relates to a method of expanding a composition of mammalian cells, comprising administering to said composition the polypeptide of the invention; or transducing/transfecting the cells with the expression vector of the invention.

In a further aspect, the invention relates to a method of differentiating a composition of mammalian cells, comprising administering to said composition the polypeptide of the invention; or transducing/transfecting the cells with the expression vector of the invention.

In a further aspect, the invention relates to an antibody capable of binding to a polypeptide of the invention.

In a further aspect, the invention relates to an immunoconjugate comprising the antibody of the invention and a conjugate selected from the group consisting of: a cytotoxic agent such as a chemotherapeutic agent, a toxin, or a radioactive isotope; a member of a specific binding pair, such as avidin or streptavidin or an antigen; an enzyme capable of producing a detectable product.

In a further aspect, the Invention relates to an isolated polypeptide having an amino acid sequence selected from the group consisting of SEQ ID No. 5 and 11 and variants of said polypeptides, wherein any amino acid specified in the chosen sequence is changed to a different amino acid, provided that no more than 15 of the amino acid residues in the sequence are so changed.

In a further aspect, the invention relates to an isolated polypeptide having an amino acid sequence selected from the group consisting of SEQ ID No 6 and 12, and variants of said polypeptides, wherein any amino acid specified in the chosen sequence is changed to a different amino acid, provided that no more than 15 of the amino acid residues in the sequence are so changed.

In a further aspect, the invention relates to an isolated polypeptide having an amino acid sequence of AA₁₄-AA₁₃₉ of SEQ ID No. 16, and variants of said polypeptide, wherein any amino acid specified in the chosen sequence is changed to a different amino acid, provided that no more than 15 of the amino acid residues in the sequence are so changed.

In a further aspect, the Invention relates to an isolated polypeptide having an amino acid sequence of SEQ ID No 20, and variants of said polypeptides, wherein any amino acid specified in the chosen sequence is changed to a different amino acid, provided that no more than 15 of the amino acid residues in the sequence are so changed.

In a further aspect, the invention relates to an isolated polypeptide having an amino acid sequence of SEQ ID No 21, and variants of said polypeptides, wherein any amino acid specified in the chosen sequence is changed to a different amino acid, provided that no more than 15 of the amino acid residues in the sequence are so changed.

In a further aspect, the invention relates to an isolated polynucleotide coding for a polypeptide according to one of the three preceding aspects of the invention.

In a further aspect, the invention relates to a method of preventing apoptosis in a neuronal cell comprising contacting a neuronal cell with an effective amount of the polypeptide of the invention; or the isolated nucleic acid sequence of the invention; or the expression vector of the invention.

### FIGURES

FIG. 1. Clustal W (1.82) alignment of the mature human and mouse Lm) peptides of the growth factor family, Cys10. In the alignments, conserved regions are shown in bold and variable regions in grey. 1A shows an alignment based on the four most similar members of the family. 1B shows an alignment based on all five members of the family of growth factors.

FIG. 1C shows a phylogenetic tree of the human protein family. The tree was generated by using Multi-Way Alignment (Align Plus 5 version 5.03, Scientific and Educational Software) and BLOSUM62 as scoring Matrix. The branch length is proportional to the difference between the sequences. The corresponding full length protein sequences are shown in Table 1.

FIG. 2. Output for human NsG31 (SEQ ID No. 3) from signal peptide prediction server SignalP v.2.0. Graphs shown for neural network based method (panel A) and hidden Markov model method (panel B).

FIG. 3. Prediction output from the ProtFun 2.1 protein function prediction algorithm for human NsG31 (SEQ ID No. 3), human NsG31-long (SEQ ID No. 15), and mouse NsG31 (SEQ ID No. 9).

FIG. 4. Clustal W (1.82) alignment of human NsG31 (SEQ ID No. 3) to mouse NsG31 (SEQ ID No 9). The signal sequence is shown in bold.

FIG. 5 shows the relative expression of NsG31, as measured by quantitative RT-PCR, (relative to tissue with the lowest expression) assuming same amounts of cDNA were synthesized from equal amounts of total RNA used for the cDNA step. For experimental details see Example 4a.

Upper panel—data obtained with primers NsG1129/1130 amplifying only the long form, NsG31-long.
Lower panel—data obtained with primers NsG131/1132 amplifying both forms of NsG31.

FIG. 6 shows the relative expression of NsG31, as measured by quantitative RT-PCR, normalised to β₂-microglobulin (relative to tissue with the lowest normalized expression). Results should be interpreted with caution as β₂-microglobulin expression levels vary between some tissues. For experimental details see Example 4a.

Upper panel—data obtained with primers NSG1129/1130 amplifying only the long form, NsG31-long
Lower panel—data obtained with primers NsG1131/1132 amplifying both forms of NsG31.

FIG. 7 shows the relative expression of NsG31, as measured by quantitative RT-PCR, (relative to tissue with the lowest expression) assuming same amounts of cDNA were synthesized from equal amounts of poly(A) RNA used for the cDNA step. For experimental details see Example 4a.

Upper panel—data obtained with primers NsG1129/1130 amplifying only the long form of NsG31.
Lower panel—data obtained with primers NSG1131/1132 amplifying both forms of NsG31.

FIG. 8 shows the relative expression of NsG31, as measured by quantitative RT-PCR, normalised to β2-microglobulin (relative to tissue with the lowest normalized expression). For experimental details see Example 4a.

Upper panel—data obtained with primers NsG1129/1130 amplifying only the long form, NsG31-long.
Lower panel—data obtained with primers NsG1131/1132 amplifying both forms of NsG31.

FIG. 9. Gene structure of human NsG31 leading to two splice variants of NsG31: NsG31 and NsG31-long.

FIG. 10. Human NsG31 cDNA (SEQ ID No. 2) and encoded polypeptide (SEQ ID No. 3).

FIG. 11. Mouse NsG31 cDNA (SEQ ID No. 8) and encoded polypeptide (SEQ ID No 9).

FIG. 12. Human NsG31-long cDNA (SEQ ID No. 14) and encoded polypeptide (SEQ ID No. 15).

FIG. 13 shows the relative expression of mGAPDH (panel A) as measured by quantitative RT-PCR (relative to tissue with the lowest expression) assuming same amounts of cDNA were synthesized from equal amounts of total RNA used for the cDNA step. In panel B and C is shown the expression of mALDH1A1 (panel B) and mOTX2 (panel C) has been normalised to the expression of mGADPH and is shown relative to the tissue with the lowest normalised expression. The expression is shown for regions of the developing mouse brain. For details, see example 13. Legend: dorsal forebrain (DFB), ventral forebrain (VFB), ventral mesencephalon (VM), dorsal mesencephalon (DM), spinal cord (SC), cortex (CTX), medial and lateral ganglionic eminences (MGE/LGE), cerebellum (Cb). E10.5, E11.5, and E13.5: embryo days (or days post conception) 10.5, 11.5 and 13.5 respectively. P1: one day post-natum. Ad: adult.

FIG. 14 shows the relative expression of mGDNF In regions of the developing mouse brain. The expression is measured by quantitative RT-PCR normalised to the expression of mGAPDH relative to tissue with the lowest normalised expression. For details, see example 13. Legend as in FIG. 13.

FIG. 15. shows the relative expression of mNsG31 in regions of the developing mouse brain. The expression is measured by quantitative RT-PCR normalised to the expression of mGAPDH relative to tissue with the lowest normalised expression. For details, see example 13a. Legend as in FIG. 13.

FIG. 16. Output for human NsG29 (SEQ ID No. 18) from signal peptide prediction server SignalP v.2.0. Graphs shown for neural network based method (panel A) and hidden Markov model method (panel B).

FIG. 17. Prediction output from the ProtFun 2.1 protein function prediction algorithm.

FIG. 18. Clustal W (1.82) alignment of human NsG29 (SEQ ID No. 18) to mouse NsG29 (SEQ ID No 23). The signal sequence is shown in bold.

FIG. 19 shows in the upper panel the relative expression of NsG29, as measured by quantitative RT-PCR, (relative to tissue with the lowest expression) assuming same amounts of cDNA were synthesized from equal amounts of total RNA used for the cDNA step. For experimental details, see Example 4b.

FIG. 19 shows in the Lower panel the relative expression of NsG29 normalised to β2-microglobulin, as measured by quantitative RT-PCR, (relative to tissue with the lowest normalized expression). Results should be interpreted with caution as β2-microglobulin expression levels vary between some tissues. For experimental details, see Example 4b.

FIG. 20 shows in the upper panel, the relative expression of NsG29, as measured by quantitative RT-PCR, (relative to tissue with the lowest expression) assuming same amounts of cDNA were synthesized from equal amounts of poly(A) RNA used for the cDNA step. For experimental details, see Example 4b.

FIG. 20 shows in the Lower panel, the relative expression of NsG29 normalised to β2-microglobulin, as measured by quantitative RT-PCR, (relative to tissue with the lowest normalized expression). For experimental details, see Example 4b.

FIG. 21. Human NsG29 cDNA (SEQ ID No.17) and encoded protein (SEQ ID No. 18).

FIG. 22. Mouse NsG29 cDNA (SEQ ID No 22) and encoded protein (SEQ ID No. 23).

FIG. 23. shows the relative expression of mNsG29 in regions of the developing mouse brain. The expression is measured by quantitative RT-PCR normalised to the expression of mGAPDH relative to tissue with the lowest normalised expression. For details, see example 13b. Legend as in FIG. 13.

### DEFINITIONS

NsG31 and NsG29, as used herein, refers to polypeptides having the amino acid sequences of substantially purified NsG29 and NsG31 (including NsG31-long) obtained from any species, particularly mammalian, including chimpanzee, bovine, ovine, porcine, murine, equine, and preferably human, from any source whether natural, synthetic, semi-synthetic, or recombinant. The term also refers to biologically active fragments of NsG29 and NsG31 obtained from any of these species, as well as to biologically active sequence variants of these and to proteins subject to posttranslational modifications. In the following, NsG31 is intended to include both human NsG31 and human NsG31-long, unless one of the forms is specifically referred to.

Growth factor characteristics as used herein define sequence-related features similar to those of classical growth factors, which are secreted proteins acting on a target cell through a receptor to cause one or more of the following responses in the target cell: growth, proliferation, differentiation, survival, regeneration, migration, regain of function, improvement of function.

According to one embodiment of the invention, “treatment”, “therapy”, and “medical use” is intended to cover prophylaxis. “Treatment”, “therapy” and “medical use” may also cover inhibition of a disease or disorder, protection against a disease or disorder, and/or prevention (not absolute) of a disease or disorder. “Treatment”, “therapy” and “medical use” may also comprise curative, ameliorative, and/or symptomatic treatment, therapy and medical use.

An “allele” or “allelic sequence”, as used herein, is an alternative form of the gene encoding NsG29 and NsG31. Alleles may result from at least one mutation in the nucleic acid sequence and may result in altered mRNAs or polypeptides whose structure or function may or may not be altered. Any given natural or recombinant gene may have none, one, or many allelic forms. Common mutational changes, which give rise to alleles, are generally ascribed to natural deletions, additions, or substitutions of nucleotides. Each of these types of changes may occur alone, or in combination with the others, one or more times in a given sequence.

A “deletion”, as used herein, refers to a change in the amino acid or nucleotide sequence and results in the absence of one or more amino acid residues or nucleotides.

An “insertion” or “addition”, as used herein, refers to a change in an amino acid or nucleotide sequence resulting in the addition of one or more amino acid residues or nucleotides, respectively, as compared to the naturally occurring molecule.

The terms “specific binding” or “specifically binding”, as used herein, refers to the high affinity interaction between a protein or peptide and a binding molecule such as an antibody and a receptor or fragments thereof. The interaction is dependent upon the presence of a particular structure (i.e., the antigenic determinant or epitope) of the protein recognized by the binding molecule. For example, if an antibody is specific for epitope “A”, the presence of a protein containing epitope A (or free, unlabeled A) in a reaction containing labelled “A” and the antibody will reduce the amount of labelled A bound to the antibody.

The term “substantially purified”, as used herein, refers to nucleic or amino acid sequences that are removed from their natural environment, isolated or separated, and are at least 60% free, preferably 75% free, and most preferably 90% free from other components with which they are naturally associated.

A “substitution”, as used herein, refers to the replacement of one or more amino acids or nucleotides by different amino acids or nucleotides, respectively.

“Sequence identity”: the determination of percent identity between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the BLASTN and BLASTP programs of Altschul, et al. (1990) J. Mol. Biol. 215:403-410.

In order to characterize the identity, subject sequences are aligned so that the highest order homology (match) is obtained. Based on these general principles the “percent identity” of two amino acid sequences may be determined using the BLASTP algorithm [Tatiana A. Tatusova, Thomas L. Madden: Blast 2 sequences—a new tool for comparing protein and nucleotide sequences; FEMS Microbiol. Lett. 1999 174 247-250], which is available from the National Center for Biotechnology Information (NCBI) web site (http://www.ncbi.nlm.nih.gov), and using the default settings suggested here (i.e. Matrix=Blosum62; Open gap=11; Extension gap=1; Penalties gap x_dropoff=50; Expect=10; Word size=3; Filter on). The BLAST algorithm performs a two-step operation by first aligning two sequences based on the settings and then determining the % sequence identity in a range of overlap between two aligned sequences. In addition to % sequence identity, BLASTP also determines the % sequence similarity based on the settings.

In order to characterize the identity, subject sequences are aligned so that the highest order homology (match) is obtained. Based on these general principles, the “percent identity” of two nucleic acid sequences may be determined using the BLASTN algorithm [Tatiana A. Tatusova, Thomas L. Madden: Blast 2 sequences—a new tool for comparing protein and nucleotide sequences; FEMS Microbiol. Lett. 1999 174 247-250], which is available from the National Center for Biotechnology Information (NCBI) web site (http://www.ncbi.nlm.nih.gov), and using the default settings suggested here (i.e. Reward for a match=1; Penalty for a match=−2; Strand option=both strands; Open gap=5; Extension gap=2; Penalties gap x_dropoff=50; Expect=10; Word size=11; Filter on). The BLASTN algorithm determines the % sequence identity in a range of overlap between two aligned nucleotide sequences.

Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the FASTA sequence alignment software package (Pearson W R, Methods Mol Biol, 2000, 132:185-219). Align calculates sequence identities based on a global alignment. Align0 does not penalise to gaps in the end of the sequences. When utilizing the ALIGN og Align0 program for comparing amino acid sequences, a BLOSUM50 substitution matrix with gap opening/extension penalties of −12/−2 is preferably used.

### DETAILED DESCRIPTION OF THE INVENTION

### I The Cys10 Family of Growth Factors

The present invention in one aspect relates to medical use of Nsg29 and NsG31 and to bioactive fragments of Nsg29 and NsG31. NsG29 and NsG31 are distinct members of a family of growth factors in the following referred to as the Cys10 family (referring to the 10 cysteines conserved among four of the five family members).

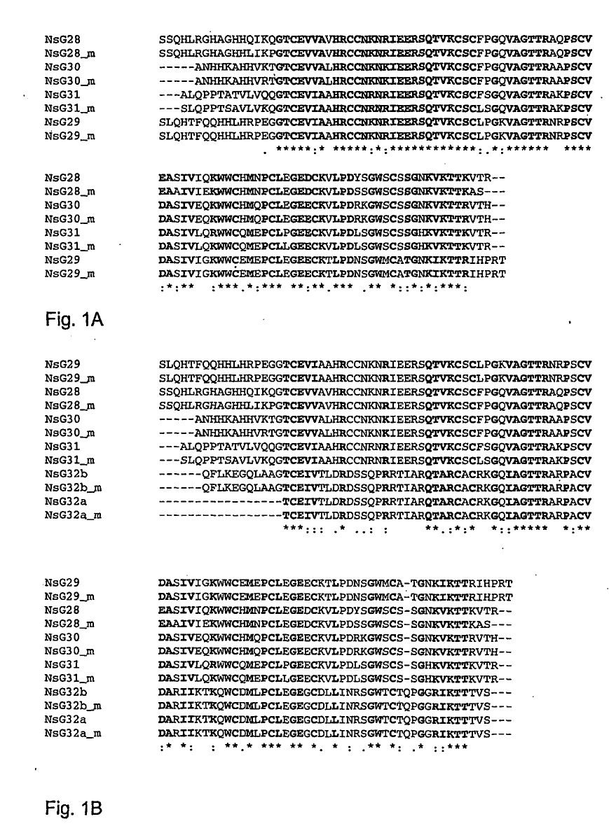

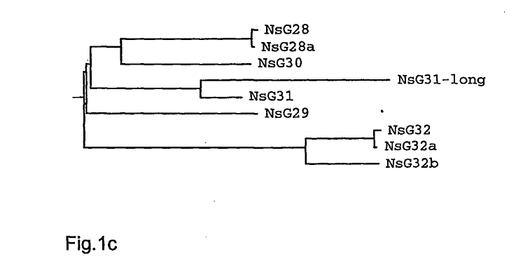

The five members of the family are referred to as NsG28, NsG29, NsG30, NsG31, and NsG32 (See Table 1). For NsG28, there are two alternative start codons in human beings, referred to as NsG28 and NsG28a. NsG31 exists as two different splice variants with different length. The long form NsG31-long (IPI00174927.1 REFSEQ_XP:XP_{—}209222 Tax_Id=9606 similar to hypothetical protein) differs from the other members of the family from position 89 to the C-terminal as a result of a frameshift. NsG32 also exists as two different splice variants, called NsG32a and NsG32b. For the NsG32a splice variant there are two possible start codons. The difference between NsG32a and NsG32b results in different signal peptides and also in different mature proteins, both of which are shown in FIG. 1B.

TABLE 1
Full length Cys10 proteins.

Human NsG28, >IPI00065186.1 (ver. 2.24)
MRSPRMRVCA KSVLLSHWLF LAYVLMVCCK LMSASSQHLR
GHAGHHQIKQ GTCEVVAVHR CCNKNRIEER SQTVKCSCFP
GQVAGTTRAQ PSCVEASIVI QKWWCHMNPC LEGEDCKVLP
DYSGWSCSSG NKVKTTKVTR
Human NsG28a, >IPI00065186.1.modi N-terminal
predicted using NetStart (ver. 2.24)
MRVCAKSVLL SHWLFLAYVL MVCCKLMSAS SQHLRGHAGH
HQIKQGTCEV VAVHRCCNKN RIEERSQTVK CSCFPGQVAG
TTRAQPSCVE AAIVIQKWWC HMNPCLEGED CKVLPDYSGW
SCSSGNKVKT TKVTR
Mouse NsG28a, >IPI00311118.3 (ver. 1.23)
MRVCAKWVLL SRWLVLTYVL MVCCKLMSAS SQHLRGHAGH
HLIKPGTCEV VAVHRCCNKN RIEERSQTVK CSCFPGQVAG
TTRAQPSCVE AAIVIEKWWC HMNPCLEGED CKVLPDSSGW
SCSSGNKVKT TKVTR
Human NsG29, >IPI00376089.1 (ver. 2.29)
MAMVSAMSWV LYLWISACAM LLCHGSLQHT FQQHHLHRPE
GGTCEVIAAH RCCNKNRIEE RSQTVKCSCL PGKVAGTTRN
RPSCVDASIV IGKWWCEMEP CLEGEECKTL PDNSGWMCAT
GNKIKTTRIH PRT
Mouse NsG29, >IPI00380407.1 (ver. 1.20)
MAMVSAMSWA LYLWISACAM LLCHGSLQHT FQQHHLHRPE
GGTCEVIAAH RCCNKNRIEE RSQTVKCSCL PGKVAGTTRN
RPSCVDASIV IGKWWCEMEP CLEGEECKTL PDNSGWMCAT
GNKIKTTRIH PRT
Human NsG30, >IPI00166553.1 (ver. 2.24)
MSKRYLQKAT KGKLLIIIFI VTLWGKVVSS ANHHKAHHVK
TGTCEVVALH RCCNKNKIEE RSQTVKCSCF PGQVAGTTRA
APSCVDASIV EQKWWCHMQP CLEGEECKVL PDRKGWSCSS
GNKVKTTRVT H
Mouse NsG30, >IPI00338844.1 (ver. 1.22)
MNKRYLQKAT QGKLLIIIFI VTLWGKAVSS ANHHKAHHVR
TGTCEVVALH RCCNKNKIEE RSQTVKCSCF PGQVAGTTRA
APSCVDASIV EQKWWCHMQP CLEGEECKVL PDRKGWSCSS
GNKVKTTRVT H
Human NsG31, >IPI00334480.1 (ver. 2.24)
MSERVERNWS TGGWLLALCL AWLWTHLTLA ALQPPTATVL
VQQGTCEVIA AHRCCNRNRI EERSQTVKCS CFSGQVAGTT
RAKPSCVDAS IVLQRWWCQM EPCLPGEECK VLPDLSGWSC
SSGHKVKTTK VTR
Mouse NsG31, >IPI00380405.1 (ver. 1.22)
MERPTSNWSA GSWVLALCLA WLMTCPASAS LQPPTSAVLV
KQGTCEVIAA HRCCNENRIE ERSQTVKCSC LSGQVAGTTR
AKPSCVDASI VLQKWWCQME PCLLGEECKV LPDLSGWSCS
SGHKVKTTKV TR
Human NsG31-long, >IPI00174927.1 (ver. 2.24)
MSERVERNWS TGGWLLALCL AWLWTHLTLA ALQPPTATVL
VQQGTCEVIA AHRCCNRNRI EERSQTVKCS CFSGQVAGTT
RAKPSCVDDL LLAAHCARRD PRAALRLLLP QPPSSCRDGG
VRWSPACRGR SVRCSRTCRD GAAAVDTKSK PPRSHDSSWG
SRPGQERLD
Human NsG32a, >IPI00239265.1.modi, N-terminal
predicted using NetStart (ver. 2.24)
MSSTFWAFMI LASLLIAYCS QLAAGTCEIV TLDRDSSQPR
RTIARQTARC ACRKGQIAGT TRARPACVDA RIIKTKQWCD
MLPCLEGEGC DLLINRSGWT CTQPGGRIKT TTVS
Human NsG32b, >IPI00385234.1 (ver. 2.29)
MQLLKALWAL AGAALCCFLV LVIHAQFLKE GQLAAGTCEI
VTLDRDSSQP RRTIARQTAR CACRKGQIAG TTRARPACVD
ARIIKTKQWC DMLPCLEGEG CDLLINRSGW TCTQPGGRIK
TTTVS
Human NsG32a alternative start codon,
>IPI00239265.1.(ver. 2.24)
MAPSPRTGSR QDATALPSMS STFWAFMILA SLLIAYCSQL
AAGTCEIVTL DRDSSQPRRT IARQTARCAC RKGQIAGTTR
ARPACVDARI IKTKQWCDML PCLEGEGCDL LINRSGWTCT
QPGGRIKTTT VS
Mouse NsG32a, >IPI00224493.1.modi N-terminal
predicted using NetStart (ver. 1.19)
MSSTFWAFMI LASLLIAYCS QLAAGTCEIV TLDRDSSQPR
RTIARQTARC ACRKGQIAGT TRARPACVDA RIIKTKQWCD
MLPCLEGEGC DLLINRSGWT CTQPGGRIKT TTVS
Mouse NsG32b, >IPI00128075.1 (ver. 1.20)
MQLLKALWAL AGAALCCFLV LVIHAQFLKE GQLAAGTCEI
VTLDRDSSQP RRTIARQTAR CACRKGQIAG TTRARPACVD
ARIIKTKQWC DMLPCLEGEG CDLLINRSGW TCTQPGGRIK
TTTVS
Mouse NsG32a alternative start codon,
>IPI00224493.1.(ver. 1.19)
MAPSPRTSSR QDATALPSMS STFWAPMILA SLLIAYCSQL
AAGTCEIVTL DRDSSQPRRT IARQTARCAC RKGQIAGTTR
ARPACVDARI IKTKQWCDML PCLEGEGCDL LINRSGWTCT
QPGGRIKTTT VS

The signal peptides are shown in bold. The IPI reference numbers (IPI - The International Protein Index. The European Bioinformatics Institute) refer to the accession numbers of the sequences. The database version is shown in parentheses. The IPI databases were downloaded from ftp://ftp.ebi.ac.uk/pub/databases/IPI/

As can be seen from FIGS. 1A and 1B, the proteins are highly conserved between man and mouse. Corresponding proteins with very high conservation are found in rat, and there is also evidence of expression of proteins with similar conserved cysteine-pattern in distantly related species such as Drosophila melanogaster, Danio rerio, Caenorhabditis elegans, Xenopus laevis, and Caenorhabditis briggsae. In these species, predicted protein fragments can be found, which fragments over lengths of 50-110 amino acids have 80-90% sequence identity to Cys10 proteins. The extremely high degree of evolutionary conservation among these species points to important functions in maintaining normal cell function in the tissues where the genes are expressed. In Xenopus laevis and in C. briggsae only two members of the Cys10 family are found. These are most similar to NsG28 and NsG32 respectively. A calculation of sequence identity and construction of a phylogenetic tree also indicate that NsG28-NsG31 have similar features and are distinct from NsG32. As predicted from the high degree of identity among NsG28, NsG29, NsG30, and NsG31, these genes may have a common ancestor gene and some functional redundancy among NsG28, NsG29, NsG30 and NsG31 may therefore be possible. Together, this indicates that there are at least two distinct functions related to the Cys10 family and that NsG28 in higher animals has evolved into four distinct members.

The percent sequence Identity calculated on the basis of the mature part of the human growth factors is shown in table 2. NsG28-NsG31 share sequence identities above 60%. NsG32 only has 8 of the 10 conserved cysteines. NsG32 is approximately 40% identical to the other members of the family. None of the members of the Cys10 family have a significant sequence identity to any characterised proteins outside this family. NsG31-long only shows partial homology to the other family members due to the frameshift located after the 6^{th} cysteine.

TABLE 2
Percent sequence identity between human mature protein
sequences using Align0 (global alignment). Scoring
matrix Blosum50. Gap penalty - 12/−2.

| | NsG29 | NsG30 | NsG31-long | NsG31 | NsG32a | NsG32b |
|---|---|---|---|---|---|---|
| NsG28a | 67.6 | 77.4 | 33.8 | 73.6 | 41.1 | 44.9 |
| NsG29 | — | 66.1 | 31.4 | 61.4 | 39.4 | 40.5 |
| NsG30 | | — | 36.1 | 72.8 | 44.1 | 46.1 |
| NsG31- | | | — | 48.2 | 19.9 | 20.5 |
| long | | | | | | |
| NsG31 | | | | — | 40.4 | 41.3 |
| NsG32a | | | | | — | 89.0 |

The Clustal W (1.82) multiple sequence alignment of the five human and mouse sequences (FIG. 1B) allows for the identification of one family fingerprint:

G-T-C-E-[V/I]-[V/I]-x(3)-R-x(5)-[R/K]-x(5)-Q-T-
[V/A]-[K/R]-C-x-C-x(2)-G-x-[V/I]-A-G-T-T-R-x(2)-P-
x-C-V-[D/E]-A-x-I-[V/I]-x(2)-[K/R]-x-W-C-x-M-x-P-
C-L-x-G-E-x-C-x(2)-L-x(4)-G-W-x-C-x(2-3)-G-x-
[K/R]-[V/I]-K-T-T

It is believed that amino acids, which are fully or partly conserved in this family fingerprint, are important for biological activity, mainly because they are believed to be important for the secondary and/or tertiary structure of the proteins. Non-conserved residues (marked as x) can probably be substituted without affecting biological activity, in particular if the substitution is a conservative substitution or to a residue found at the same or corresponding position in another Cys10 protein. Only NgG32a does not have the initial glycine residue. The same core sequence with minor modifications can be found in Drosophila melanogaster, X. laevis, C. briggsae, and Danio rerio. The present inventors therefore in one embodiment predict that the core sequence from GTCE to KTT is the bioactive core sequence, which can be used to elicit the same biological effects as mature Cys10 proteins.

From the alignment it can be seen, that NsG32 only has 8 of the 10 cysteines, that are conserved in the other members of the family and that the mature NsG32 protein in the region, where the other members have a double cysteine, has a stretch of amino acids, which differs from all the other members of the family. Another Clustal W (1.82) alignment of only the most similar members of the family, NsG28-NsG31 (FIG. 1A) gives another family fingerprint with a higher degree of conservation across the sub-family:

G-T-C-E-V-[V/I]-A-x-H-R-C-C-N-[K/R]-N-[R/K]-I-E-E-
R-S-Q-T-V-K-C-S-C-x(2)-G-x-V-A-G-T-T-R-x(2)-P-S-C-
V-[D/E]-A-x-I-V-x(2)-[K/R]-W-W-C-x-M-x-P-C-L-x-G-
E-[E/D]-C-K-x-L-P-D-x(2)-G-W-x-C-x-[S/T]-G-x-K-
[V/I]-K-T-T-[R/K]

In another embodiment, the present inventors predict that a NsG28-NsG31 polypeptide comprising this conserved (GTCEV - - - KTT[RK]) region can elicit the biological effects of mature Cys10 proteins. It is believed that amino acids, which are fully or partly conserved in this family fingerprint, are important for biological activity, mainly because they are believed to be important for the secondary and/or tertiary structure of the proteins. Non-conserved residues (marked as x) can probably be substituted without affecting biological activity, in particular if the substitution is a conservative substitution or to a residue found at the same or corresponding position in another Cys10 protein.

The conserved cysteines may participate in forming intra- and Inter-molecular cystine bridges and may thus play an important role in the secondary and tertiary structure of the proteins, Just as is the case for other growth factors with a conserved cysteine pattern (e.g. the TGF-beta family). It is possible that Cys10 proteins may form both homo- and heterodimers.

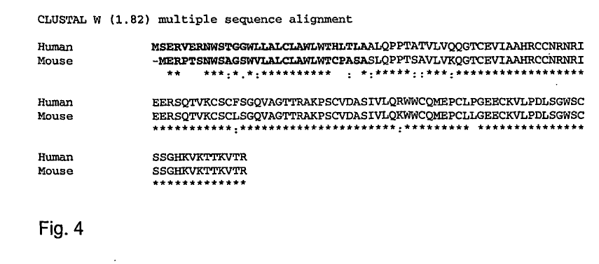

The ClustalW alignments in FIGS. 1 and 4 can be used to identify those parts of the growth factors in which mutations can be made without substantially altering the biological function. In particular it is expected that a residue, which is not fully conserved among mouse, rat, and man (FIG. 4) in the same Cys10 member can be replaced with a residue found at the same position in another species. Furthermore, it is expected that a residues that are non-conserved among the Cys10 members (FIGS. 1A and 1B) can be substituted with a residue found in another Cys10 member. In particular, it is possible that residues at positions that are non-conserved within the NsG28-NsG31 group can be substituted with each other.

Apart from sharing a high degree of sequence Identity, all members and variants of the Cys10 family are predicted to belong to the gene ontology class, growth factor and/or hormone (ProtFun prediction, see Example 2). Expression of all five members seems to be restricted to the central and peripheral nervous system including the eye, but each individual member shows differential expression in sub-regions.

Until now, none of the members of the Cys10 family of growth factors have been characterised and none of the members have shown a biological activity in any in vitro or in vivo assay. The proteins have not been produced, purified or characterised. In the prior art they are predicted as hypothetical proteins, and there is no credible prediction available about their biological function.

### IIa NsG31

NsG31 is a 133 amino acid secreted growth factor protein or hormone. The mouse (IPI00380405.1 version 1.22) homologue has a full length of 132 amino acids with a % sequence identity of 85.7.

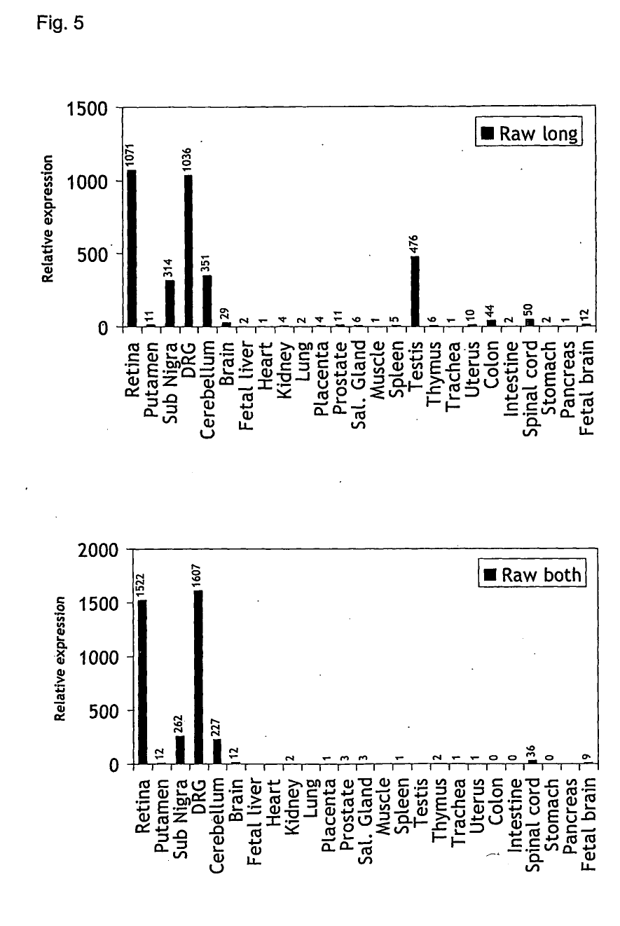

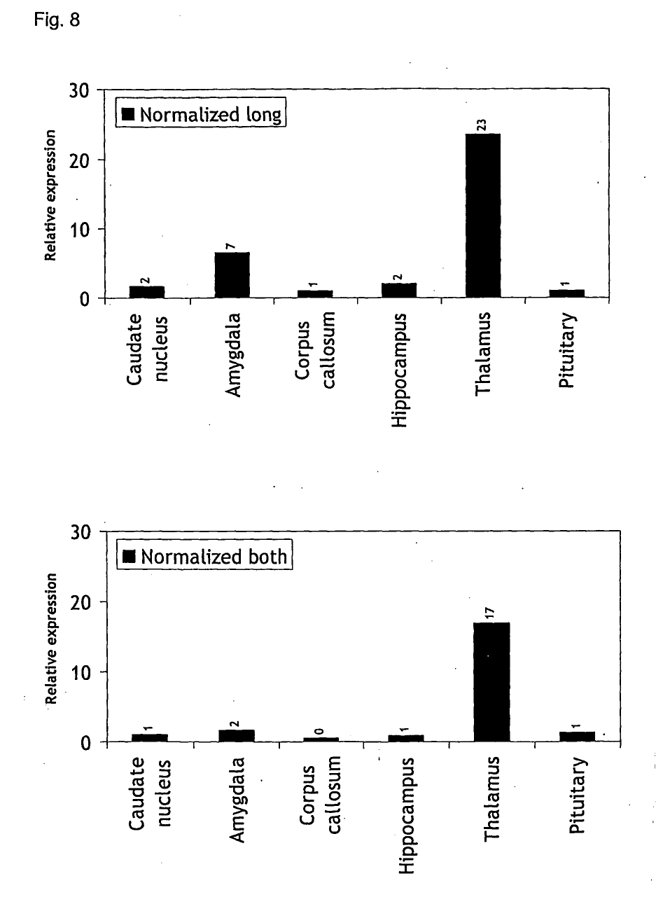

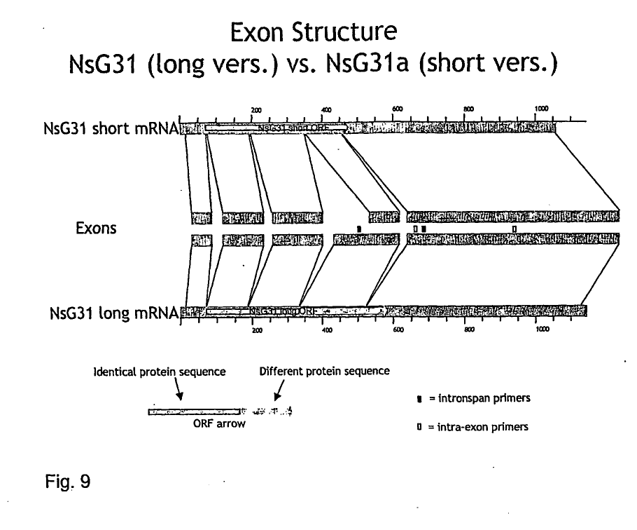

In human beings an alternative splice form is present. In the alternative splice form, which is referred to in the present application as NsG31-long, exon 4 contains an additional 68 nucleotides (FIG. 9). As these additional 68 nucleotides are present in the open reading frame, the insertion results in a frameshift compared to human NsG31 (see FIG. 9). It has been possible to amplify both cDNAs from a retina cDNA library with a primerpair spanning from exon 1 to exon 5. Both ORFs have been cloned and are inserted into lentivirus. The quantitative expression analysis has been performed with two primerpairs, one of which amplifies only the NsG31-long form (intronspan primer in FIG. 9). As can be seen from FIGS. 5, 6, 7, and 8 (upper panels), the NsG31-long form is expressed at significant levels. The expression pattern seen with a primer pair amplifying both the long and the short form (FIG. 5-8 lower panels), is almost the same. There is thus evidence that both forms of human NsG31 are transcribed. A similar splice variation is not found in mouse.

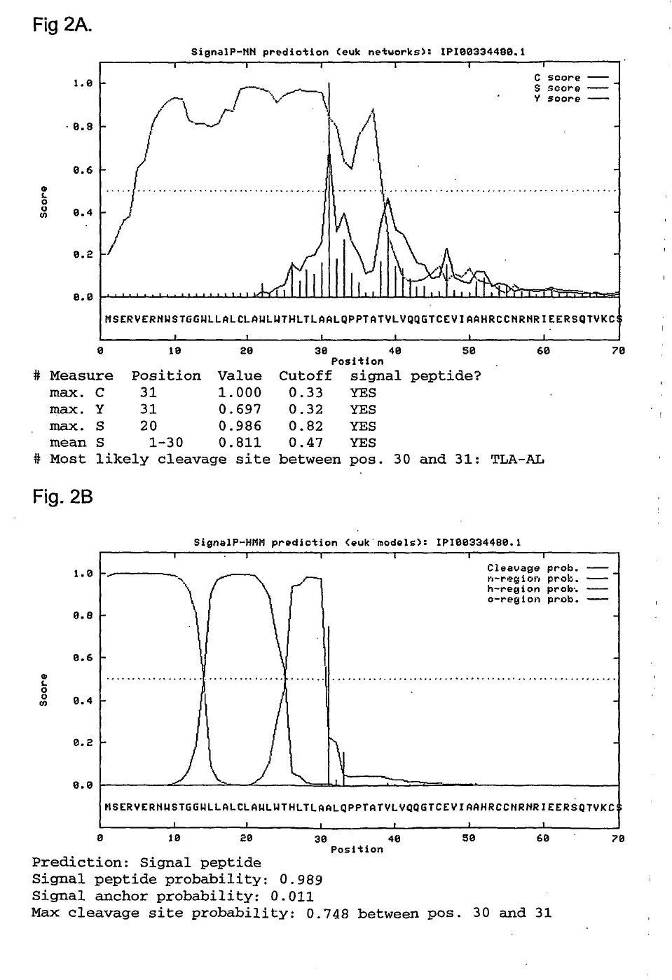

Human NsG31 contains an N-terminal signal peptide sequence of 30 amino acids, which is cleaved at the sequence motif TLA-AL. This signal peptide cleavage site is predicted by the SignalP method (Nielsen et al., 1997) and the output graph shown in FIG. 2. A signal peptide cleavage site is found at a similar location in the mouse NsG31 after position 29. The same signal sequence is present in the human alternative splice form NsG31-long.

As it is known in the art, signal peptide processing is not always exactly as predicted and actual cleavage may vary from case to case. Thus, it is expected that the N-terminal of mature NsG31 may vary by one to two or three amino acids from the predicted cleavage site. The actual N-terminal of mature NsG31 can be verified experimentally by C-terminal tagging with e.g. a his-tag, subsequent purification using a poly-his specific antibody or purification on a Ni column, and finally N-terminal sequencing of the purified mature peptide.

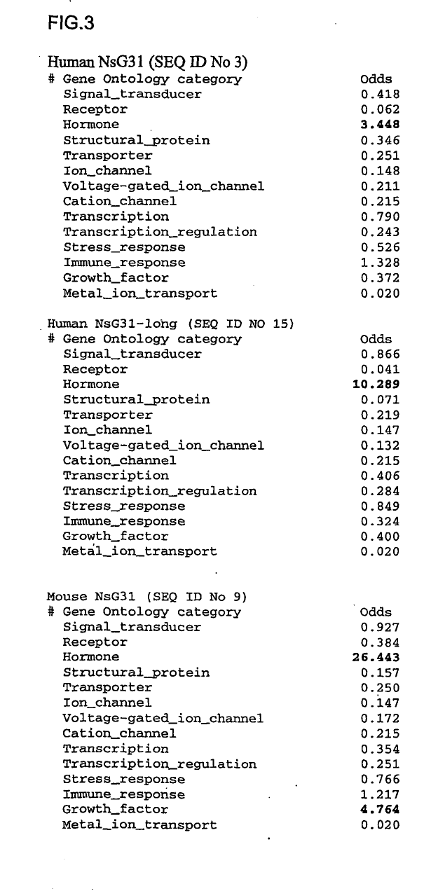

NsG31 belongs to the category of proteins acting as hormones or growth factors. This notion is supported by predictions by the ProtFun protein function prediction server (Jensen et al., 2002 & 2003), which provides odds scores above 1 for the hormone category as shown in FIG. 3. The mouse homologue provides odds scores above 1 for both the hormone and the growth factor category. Human NsG31-long also provides odds score above 1 for the hormone category.

Results of the quantitative expression analysis in human tissues are shown in FIGS. 5, 6, 7, and 8. The expression analyses show:

High and intermediate expression in: Retina, Dorsal Root Ganglion, Cerebellum, Substantia Nigra, testis, and Thalamus
Low expression in: Brain, Foetal Brain, Colon, Foetal Liver, Heart, Kidney, Lung, Placenta, Putamen, Prostate, Salivary Gland, Skeletal muscle, spleen, Thymus, Trachea, Uterus, Small Intestine, Spinal Cord, Stomach, Pancreas, Amygdala, Caudate nucleus, Corpus Callosum, Hippocampus, and Pituitary

These conclusions were drawn from data obtained from quantitative RT-PCR analyses performed with primer set NsG1129/1130 detecting NsG31-long. The same conclusions were drawn from data obtained with another primerset (NsG1131/1132) detecting both forms. The tissues for which no relative expression data are shown in the lower panels of FIGS. 4-5, have not been tested in the PCR which detects both the long and short form of human NsG31.

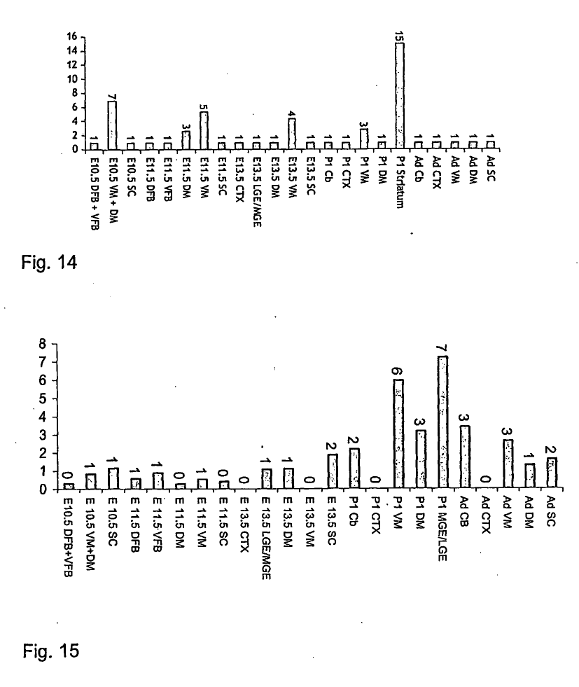

The results of quantitative expression analysis in the developing mouse CNS are shown in FIG. 15. These results should be interpreted with care, as the absolute expression levels measured with this primerset is much lower than the expression measured in human CNS. Expression in the developing mouse CNS peaks at P1 in the ventral mesencephalon and in the striatum. i.e. at around the time of terminal differentiation in these tissues and around the time of differentiation and termination of the projections between the striatum and the ventral mesencephalon.

Unlike structural proteins, growth factors are involved in cell signalling and in various functions such as growth, proliferation, differentiation, survival, regeneration, migration, regain of function and improvement of function. Therefore, growth factors can be administered and be used to exert a therapeutic effect. Based on the tissue specific expression, the fact that NsG31 is predicted to be a secreted growth factor, and the fact that the closely related growth factor, NsG30 has shown an effect on survival in a cell line with neuronal potential (PC12 assay corresponding to Example 5 of the present invention), NsG31 is contemplated for use in treating disorders of the nervous system in general (based on the nervous system specific expression), in particular Parkinson's disease (based on the expression in human substantia nigra and thalamus, and based on the expression in the developing mouse VM and striatum), Huntington's disease (based on expression in human substantia nigra, and the developing mouse striatum and VM), cerebellar disorders (based on expression in cerebellum), peripheral neuropathies (based on expression in dorsal root ganglion), thalamic pain and essential tremor (based on expression in the thalamus), testicular disorders and male contraception (based on expression in the testicles), and retinopathies (based on expression in retina). The function for the various indications can be verified in in vitro and in vivo assays as described in the examples.

Likewise, expression of therapeutically relevant secreted growth factors including GDNF, NGF, and Neublastin (Artemin) is found in target areas of the neurological and/or testicular disorder they may be used to treat.

The therapeutic effect of NsG31 may be mediated through an effect on growth, proliferation, regeneration, regain of function, improvement of function, survival, migration, and/or differentiation of targeted cells.

Other members of the Cys10 family, NsG30, NsG32a, and NsG32b, have shown a survival enhancing effect in a cell line with neuronal potential (PC12 assay described in Example 5 herein). The assay tests the ability of the factors to protect a neuronal cell line against apoptotic cell death. Apoptotic cell death contributes to neuronal cell loss in the adult nervous system causing various neurological disorders like ischemic stroke, neurodegenerative diseases or brain traumata (Becker & Bonni, Prog Neurobiol, 2004 January; 72(1):1-25). Similarly, NsG28 and NsG32a have shown a small but significant effect in protecting cerebellar granule cells from potassium-depravation induced apoptosis. Taken together these data indicate that the Cys10 factors are antiapoptotic factors with an effect on the nervous system. A secreted growth factor capable of protecting neuronal cells against apoptotic cell death is therefore a candidate for treating neurological disorders. The ability of a secreted growth factor to promote survival under conditions leading to apoptosis is an indication that this factor has a similar effect in other neuronal cell types of the central and/or peripheral nervous system.

### IIb NsG29

Human NsG29 (SEQ ID No. 18) is a 133 amino acid secreted growth factor protein or hormone, which is very similar to the mouse NsG29 (SEQ ID No. 23) with a length of 133 and sequence identity of 99.2%.

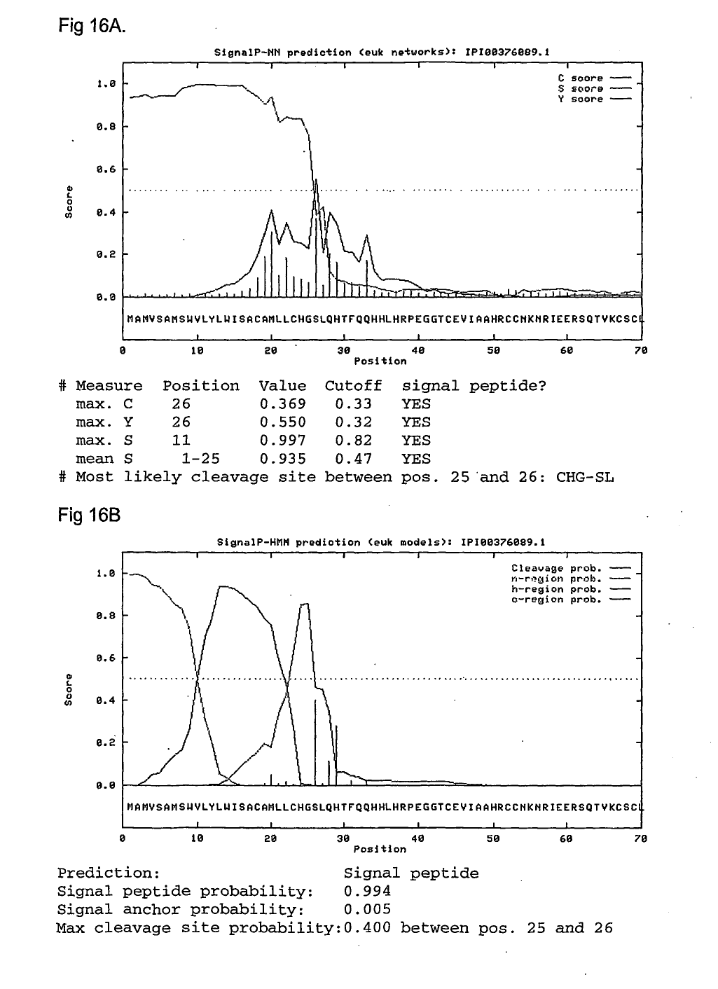

Human NsG29 contains a N-terminal signal peptide sequence of 25 amino acids which is cleaved at the sequence motif CHG-SL. This signal peptide cleavage site is predicted by the SignalP method (Nielsen et al., 1997) and the output graph shown in FIG. 16. The mouse NsG29 cleavage site is also found after position 25.

As it is known in the art, signal peptide processing is not always exactly as predicted and actual cleavage may vary from case to case. Thus, it is expected that the N-terminal of mature NsG29 may vary by one to two or three amino acids from the predicted cleavage site. The actual N-terminal of mature NsG29 can be verified experimentally by C-terminal tagging with e.g. a his-tag, subsequent purification using a poly-his specific antibody or purification on a Ni column, and finally N-terminal sequencing of the purified mature peptide.

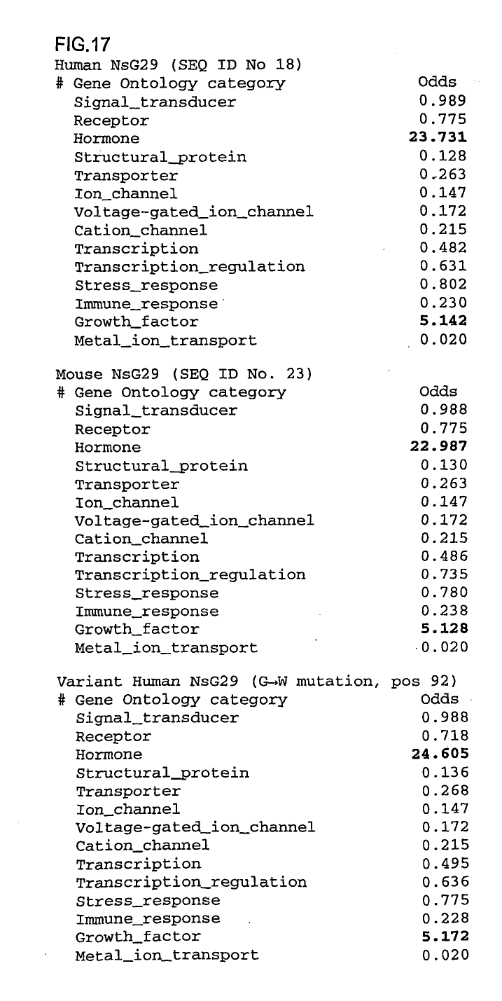

Human NsG29 belongs to the category of proteins acting as hormones or growth factors. This notion is supported by predictions by the ProtFun protein function prediction server (Jensen et al., 2002 & 2003), which provides scores above 1 for both these categories for both mouse and human NsG29 as shown in FIG. 17. Also the variant human NsG29 with a G→W mutation at position 92 provides scores above 1 for both the hormone and growth factor ontology classes.

The ProtFun method predicts protein function based on sequence-derived features as opposed to sequence similarity. Features which are important for discriminating between the ‘growth factor/hormone’ classes versus all other classes are: protein sorting potential, protein targeting potential, signal peptide potential, low complexity regions, secondary protein structure, number of negative residues and number of atoms (Jensen et al., 2003).

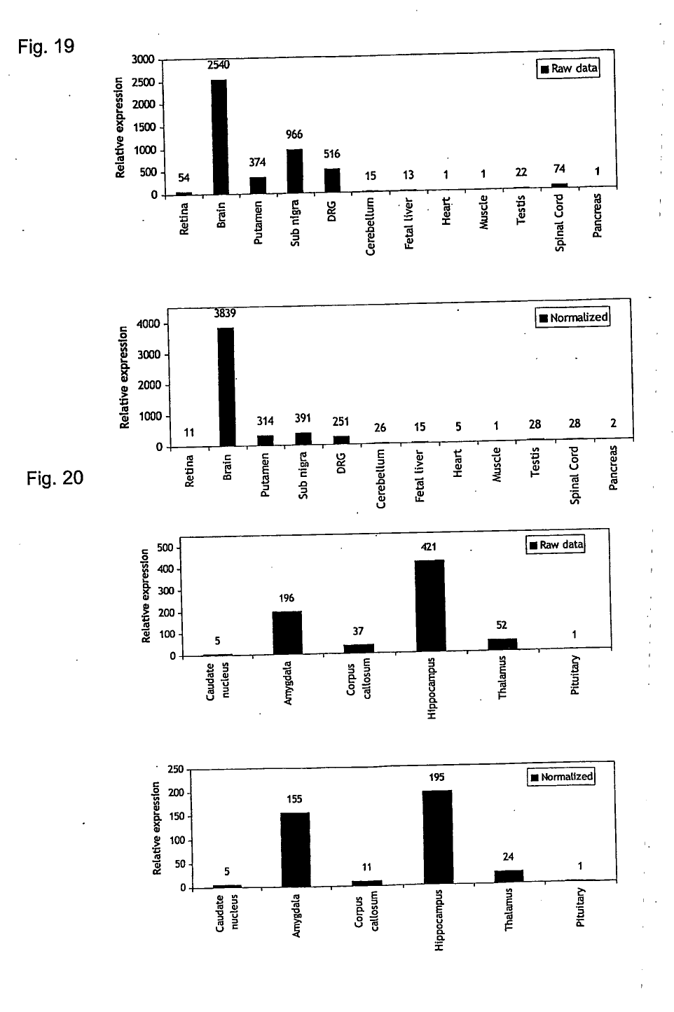

Results of the quantitative expression analysis (Example 4b) are shown in FIGS. 19 and 20. The expression analyses show:

High to moderate expression in: Brain, Substantia Nigra, Dorsal Root Ganglion, Putamen, Hippocampus, and Amygdala
Intermediate expression in: Thalamus, Corpus Callosum, and Caudate Nucleus
Low expression in: Spinal Cord, Retina, testis, cerebellum, and foetal liver
Very low or no expression in: Heart, pancreas, muscle, and pituitary gland

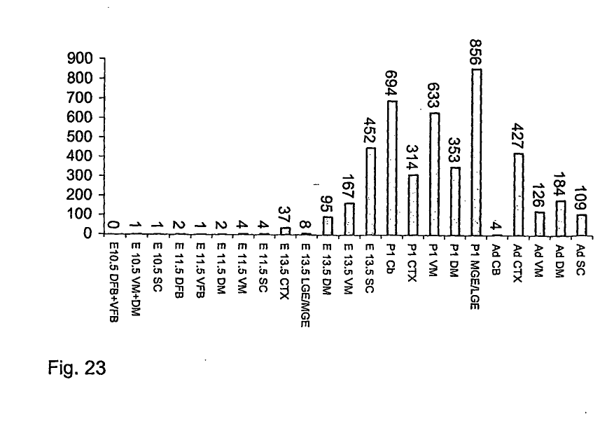

Results of the quantitative expression analysis (Example 13b; FIG. 23) in the developing mouse CNS indicate a peak in expression in various areas of the nervous system during the late foetal and neonatal period that are involved in terminal differentiation. The pattern of expression indicates a peaking in the caudal areas of the CNS, e.g. spinal cord before expression in the rostral regions. This coincides with the myelination of the nervous system. The retention of partial expression in the adult CNS may indicate a role in the myelination and maintenance of the axon sheaths and oligodendrocytes. Expression in the adult amygdale and hippocampus may indicate a role in stem cell biology and neurogenesis as these regions show extensive plasticity and stem cell proliferation during adulthood. Expression also peaks during the late foetal and neonatal period in the cerebellum and the dorsal mesencephalon. Expression in the adult mouse CNS remains relatively high except for the cerebellum, where expression is very low compared to P1.

Furthermore, a genechip analysis (Example 4b) has shown that NsG29 is expressed in the developing mesencephalon of the human embryo, indicating that the NsG29 may play a role in early foetal brain development. Expression of a growth factor in the human mesencephalon during embryo development is predictive of a possible therapeutic function in the treatment of Parkinson's Disease.

Unlike structural proteins, growth factors are involved in cell signalling and in various functions such as growth, proliferation, differentiation, survival, regeneration, migration, regain of function and improvement of function. Therefore, growth factors can be administered and be used to exert a therapeutic effect. Based on the tissue specific expression, the fact that NsG29 is predicted to be a secreted growth factor, and the fact that other Cys10 factors (human NsG28, NsG30, and NsG32) have shown a survival enhancing effect on primary neuronal cells (cerebellar granule cells) and on a cell line with neuronal potential (corresponding to the PC12 assay as described herein), NsG29 is contemplated for use in treating disorders of the nervous system in general (based on the nervous system specific expression), in particular Parkinson's disease (based on the expression in substantia nigra and in the human developing mesencephalon; and based on the expression in the developing mouse CNS), Alzheimer's Disease (based on expression in human Amygdala and Hippocampus), Huntingtons disease (based on expression in human putamen and substantia nigra; and based on the expression during mouse CNS development), cerebellar disorders (based on expression in human cerebellum and developing mouse Cerebellum), Spinal Cord injury (based on expression in the human spinal cord and the developing mouse spinal cord), ALS (based on expression in the human spinal cord and the developing mouse spinal cord), multiple sclerosis (based on the expression in the developing mouse CNS which indicates a role in myelinisation), peripheral neuropathies (based on expression in dorsal root ganglion), and retinopathies (based on expression in retina). The function for the various indications can be verified in in vitro and in vivo assays as described in the examples.

Likewise, expression of therapeutically relevant secreted growth factors including GDNF, NGF, and Neublastin (Artemin) is found in target areas of the neurological disorder they may be used to treat.

The therapeutic effect of NsG29 may be mediated through an effect on growth including proliferation, regeneration, regain of function, improvement of function, survival, migration, and/or differentiation of targeted cells.

Other members of the Cys10 family, NsG30, NsG32a, and NsG32b, have shown a survival enhancing effect in a cell line with neuronal potential (PC12 assay described in Example 5 herein). The assay tests the ability of the factors to protect a neuronal cell line against apoptotic cell death. Apoptotic cell death contributes to neuronal cell loss in the adult nervous system causing various neurological disorders like Ischemic stroke, neurodegenerative diseases or brain traumata (Becker & Bonni, Prog Neurobiol, 2004 January; 72(1):1-25). NsG28 and NsG32a have also demonstrated an antiapoptotic effect by protecting mouse cerebellar granule neurons from low calcium induced apoptosis (assay essentially as described by Nomura et al, 2001, Dev. Neurosci. 23: 145-152). One function of NsG29 inferred from the sequence homology to NsG28, NsG30, NsG32a and NsG32b is therefore an antiapoptotic effect on cellular populations from the nervous system. A secreted growth factor capable of protecting neuronal cells against apoptotic cell death is therefore a candidate for treating neurological disorders. The ability of a secreted growth factor to promote survival under conditions leading to apoptosis is an indication that this factor has a similar effect in other neuronal cell types of the central and/or peripheral nervous system.

### III NsG29 and NsG31 Polypeptides

In addition to full-length NsG29 and NsG31 (including NsG31-long), substantially full-length NsG29 and NsG31, mature NsG29 and NsG31, and to truncated forms of NsG29 and NsG31, the present invention provides for biologically active variants of the polypeptides. An NsG29 and/or NsG31 polypeptide or fragment is biologically active if it exhibits a biological activity of naturally occurring NsG29 and/or NsG31 respectively. It is to be understood that the invention relates to substantially purified Nsg29 and/or NsG31 as herein defined.

One biological activity is the ability to compete with naturally occurring NsG29 and/or NsG31 in a receptor-binding assay. Another biological activity is the ability to bind to an antibody, which is directed at an epitope, which is present on naturally occurring NsG29 and/or NsG31.

Biologically active variants may also be defined with reference to one or more of the other in vitro and/or in vivo biological assays described in the examples.

Variants can differ from naturally occurring NsG29 and NsG31 in amino acid sequence or in ways that do not involve sequence, or in both ways. Variants in amino acid sequence (“sequence variants”) are produced when one or more amino acids in naturally occurring NsG29 and NsG31 is substituted with a different natural amino acid, an amino acid derivative or non-native amino acid. Particularly preferred variants include naturally occurring NsG29 and NsG31, or biologically active fragments of naturally occurring NsG29 and NsG31, whose sequences differ from the wild type sequence by one or more conservative and/or semiconservative amino acid substitutions, which typically have minimal influence on the secondary and tertiary structure and hydrophobic nature of the protein or peptide. Variants may also have sequences, which differ by one or more non-conservative amino acid substitutions, deletions or insertions, which do not abolish the NsG29 and/or NsG31 biological activity. The Clustal W alignments in FIG. 1A, 1B, 4, and 18 can be used to predict which amino acid residues can be substituted without substantially affecting the biological activity of the protein.

Substitutions within the following group (Clustal W, ‘strong’ conservation group) are to be regarded as conservative substitutions within the meaning of the present invention
STA, NEQK, NHQK, NDEQ, QHRK, MILV, MILF, HY, FYW.
   Substitutions within the following group (Clustal W, ‘weak’ conservation group) are to be regarded as semi-conservative substitutions within the meaning of the present invention
CSA, ATV, SAG, STNK, STPA, SGND, SNDEQK, NDEQHK, NEQHRK, VLIM, HFY.

Other variants within the invention are those with modifications which increase peptide stability. Such variants may contain, for example, one or more nonpeptide bonds (which replace the peptide bonds) in the peptide sequence. Also included are: variants that include residues other than naturally occurring L-amino acids, such as D-amino acids or non-naturally occurring or synthetic amino acids such as beta or gamma amino acids and cyclic variants. Incorporation of D-instead of L-amino acids into the polypeptide may increase its resistance to proteases. See, e.g., U.S. Pat. No. 5,219,990. Splice variants are specifically included in the invention.

When the result of a given substitution cannot be predicted with certainty, the derivatives may be readily assayed according to the methods disclosed herein to determine the presence or absence of biological activity.

Non-sequence modifications may include, for example, in vivo or in vitro chemical derivatisation of portions of naturally occurring NsG29 or NsG31, as well as acetylation, methylation, phosphorylation, carboxylation, PEG-ylation, or glycosylation. Just as it is possible to replace substituents of the protein, it is also possible to substitute functional groups, which are bound to the protein with groups characterized by similar features. Such modifications do not alter primary sequence. These will initially be conservative, i.e., the replacement group will have approximately the same size, shape, hydrophobicity and charge as the original group.

Many amino acids, including the terminal amino acids, may be modified in a given polypeptide, either by natural processes such as glycosylation and other post-translational modifications, or by chemical modification techniques, which are well known in the art. Among the known modifications which may be present in polypeptides of the present invention are, to name an illustrative few, acetylation, acylation, ADP-ribosylation, amidation, covalent attachment of flavin, covalent attachment of a heme moiety, covalent attachment of a polynucleotide or polynucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Such modifications are well known to those of skill and have been described in great detail in the scientific literature. Several particularly common modifications, glycosylation, lipid attachment, sulfation, gamma-carboxylation of glutamic acid residues, hydroxylation and ADP-ribosylation, for instance, are described in most basic texts, such as, for instance, I. E. Creighton, Proteins-Structure and Molecular Properties, 2nd Ed., W. H. Freeman and Company, New York, 1993. Many detailed reviews are available on this subject, such as, for example, those provided by Wold, F., in Posttranslational Covalent Modification of Proteins, B. C. Johnson, Ed., Academic Press, New York, pp 1-12, 1983; Seifter et al., Meth. Enzymol. 182: 626-646, 1990 and Rattan et al., Protein Synthesis: Posttranslational Modifications and Aging, Ann. N.Y. Acad. Sci. 663: 48-62, 1992.

In addition, the protein may comprise a protein tag to allow subsequent purification and optionally removal of the tag using an endopeptidase. The tag may also comprise a protease cleavage site to facilitate subsequent removal of the tag. Non-limiting examples of affinity tags include a polyhis-tag, a GST tag, a HA tag, a Flag tag, a C-myc tag, a HSV tag, a V5 tag, a maltose binding protein tag, a cellulose binding domain tag. Preferably for production and purification, the tag is a polyhis-tag. Preferably, the tag is in the C-terminal portion of the protein.

The native signal sequence of NsG29 and NsG31 may also be replaced in order to increase secretion of the protein in recombinant production in other mammalian cell types.

It will be appreciated, as is well known and as noted above, that polypeptides are not always entirely linear. For instance, polypeptides may be branched as a result of ubiquitination, and they may be circular, with or without branching, generally as a result of posttranslational events, including natural processing events and events brought about by human manipulation which do not occur naturally. Circular, branched and branched circular polypeptides may be synthesized by non-translational natural processes and by entirely synthetic methods, as well and are all within the scope of the present invention.

Modifications can occur anywhere in a polypeptide, including the peptide backbone, the amino acid side-chains and the amino or carboxyl termini. In fact, blockage of the amino or carboxyl group in a polypeptide, or both, by a covalent modification, is common in naturally occurring and synthetic polypeptides and such modifications may be present in polypeptides of the present invention, as well. For instance, the amino terminal residue of polypeptides made in E. coli, prior to proteolytic processing, almost invariably will be N-formylmethionine.

The modifications that occur in a polypeptide often will be a function of how it is made. For polypeptides made by expressing a cloned gene in a host, for instance, the nature and extent of the modifications in large part will be determined by the host cell's posttranslational modification capacity and the modification signals present in the polypeptide amino acid sequence. For instance, glycosylation often does not occur in bacterial hosts such as E. coli. Accordingly, when glycosylation is desired, a polypeptide should be expressed in a glycosylating host, generally a eukaryotic cell. Insect cells often carry out the same posttranslational glycosylations as mammalian cells and, for this reason, insect cell expression systems have been developed to efficiently express mammalian proteins having native patterns of glycosylation, inter alia. Similar considerations apply to other modifications.

It will be appreciated that the same type of modification may be present to the same or varying degree at several sites in a given polypeptide. Also, a given polypeptide may contain many types of modifications.

In general, as used herein, the term polypeptide encompasses all such modifications, particularly those that are present in polypeptides synthesized by expressing a polynucleotide in a host cell.

Also included within the invention are agents, which specifically bind to a protein of the invention, or a fragment of such a protein. These agents include Ig fusion proteins and antibodies (including single chain, double chain, F_{ab} fragments, and others, whether native, humanized, primatized, or chimeric). Additional descriptions of these categories of agents are in WO 95/16709, the disclosure of which is herein incorporated by reference.

Antibodies refer to intact molecules as well as fragments thereof, such as F_{ab}, F_{(ab′)}, and Fᵥ, which are capable of binding the epitopic determinant. Antibodies that bind NsG29 or NsG31 polypeptides can be prepared using intact polypeptides or fragments containing small peptides of Interest as the immunizing antigen. The polypeptide or oligopeptide used to immunize an animal can be derived from the translation of RNA or synthesized chemically and can be conjugated to a carrier protein, if desired. Commonly used carriers that are chemically coupled to peptides include bovine serum albumin and thyroglobulin, keyhole limpet hemocyanin. The coupled peptide is then used to immunize the animal (e.g., a mouse, a rat, or a rabbit).

Humanised antibodies, as used herein, refer to antibody molecules in which amino acids have been replaced in the non-antigen binding regions in order to more closely resemble a human antibody, while still retaining the original binding ability. Humanised antibodies may be used therapeutically to treat conditions, where it is desirable to limit or block the action of NsG29 and/or NsG31.

Also included within the scope of the present invention are immunoconjugates of antibodies and conjugates selected from the group consisting of: a cytotoxic agent such as a chemotherapeutic agent, a toxin, or a radioactive isotope; a member of a specific binding pair, such as avidin, or streptavidin, or an antigen; an enzyme capable of producing a detectable product. These immunoconjugates can be used to target the conjugates to cells expressing an NsG29 and/or NsG31 receptor.

Specific antibodies to any NsG29 and/or NsG31 are also useful in immunoassays to quantify the substance for which a given antibody has specificity. Specific antibodies to an NsG29 and/or NsG31 may also be bound to solid supports, such as beads or dishes, and used to remove the ligand from a solution, either for use in purifying the protein or in clearing it from the solution. Each of these techniques is routine to those of skill in the immunological arts.

Also with the scope of the present invention are NsG29 and NsG31 fusion proteins. An Nsg29 and/or NsG31 fusion protein can be used to allow imaging of tissues which express a receptor for NsG29 and/or NsG31, or in the immunohistological or preparative methods described above for antibodies to an NsG29 and/or NsG31. Fusion proteins encompassing an NsG29 and/or NsG31 can be used to specifically target medical therapies against cells, which express an NsG29 and/or NsG31 receptor.

In the following polypeptide sequences of NsG31 are described first, followed by NsG29 polypeptide sequences.

Preferably, biological activity of naturally occurring NsG31 is the activity of human or mouse NsG31 having the amino acid sequence of SEQ ID No. 3 and 9, respectively.

In another embodiment, biological activity of naturally occurring NsG31 is the activity of NsG31-long (SEQ ID No. 15).

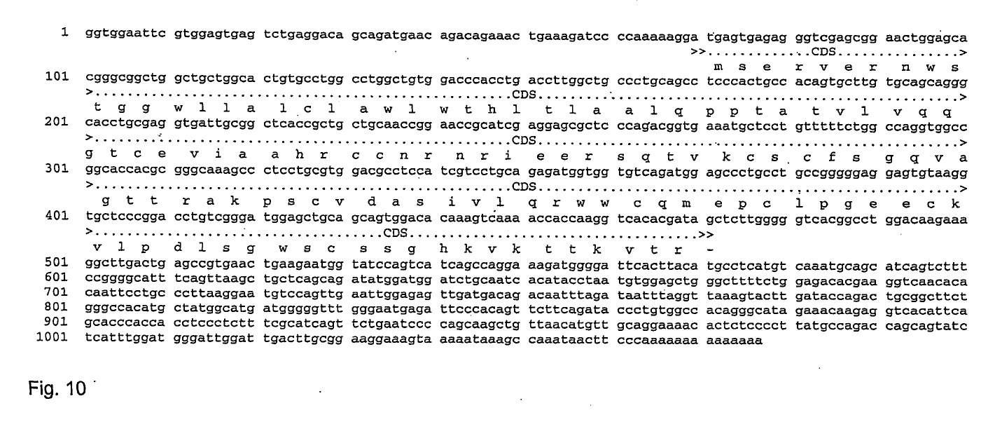

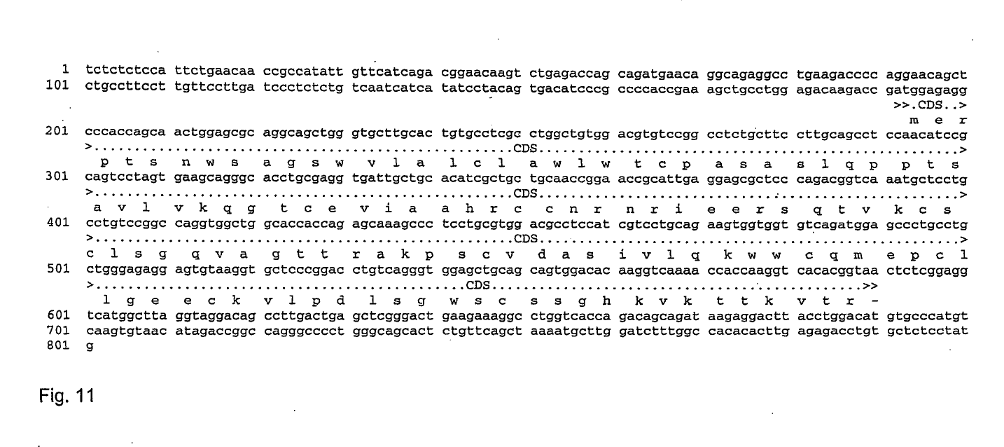

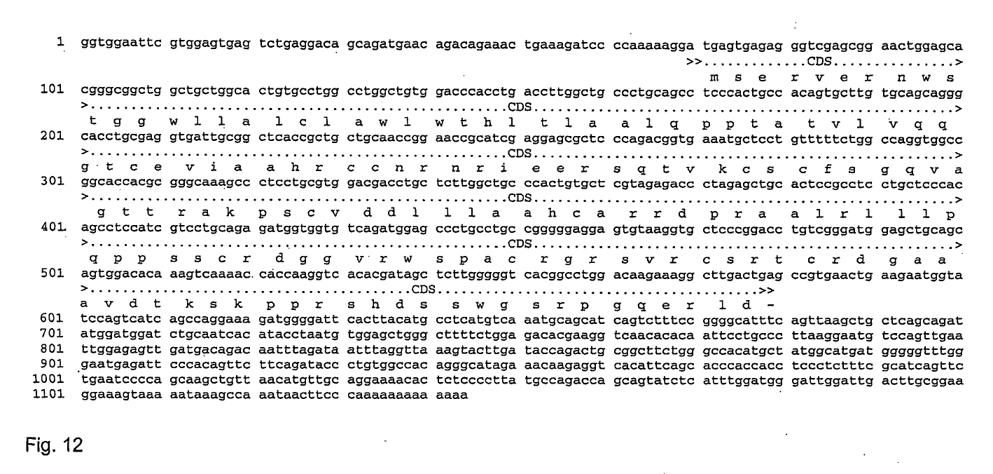

A preferred biological activity is the ability to elicit substantially the same response as in the PC12 assay described in the Examples. In this assay PC12 cells are transduced with full-length human NsG31 or NsG31-long coding sequence (FIGS. 10 and 12 respectively). By substantially the same response in the PC12 assay is intended that the number of neurite bearing cells is at least 10% of the number obtained in Example 5 (transduction with full length human NsG31 or NsG31-long), more preferably at least 20%, more preferably at least 30%, more preferably at least 40%, more preferably at least 50%, more preferably at least 60%, more preferably at least 70%, more preferably at least 75%, more preferably at least 80%, more preferably at least 85%, more preferably at least 90%. The PC12 assay may also be used to document the percentage improvement in survival over a control treatment. Substantially the same response in this context means an activity resulting in at least 10% of the improvement obtained in Example 5, more preferably at least 20%, more preferably at least 30%, more preferably at least 40%, more preferably at least 50%, more preferably at least 60%, more preferably at least 70%, more preferably at least 75%, more preferably at least 80%, more preferably at least 90%. The biological activity of a fragment or variant of NsG31 may also be higher than that of the naturally occurring NsG31.

Specific fragments of NsG31 include polypeptides having a sequence selected from the group consisting of SEQ ID No. 5 and 11, and sequence variants of said polypeptides, wherein any amino acid specified in the chosen sequence is changed to a different amino acid, provided that no more than 10 of the amino acid residues in the sequence are so changed. These isolated polypeptides constitute NsG31 polypeptides spanning from the first to the last of the 10 conserved cysteine residues of the Cys10 family. It is believed that the biological activity mainly resides in this part of the protein, and that the sequence with all the ten cysteines is required for correct folding of the protein. Preferably any changes in these peptides are to residues marked in the alignment of FIG. 1A as unconserved, weakly conserved, or strongly conserved. More preferably any changed amino acids are selected from those designated as unconserved, weakly conserved or strongly conserved in FIG. 4. Preferably any changed amino acid is changed to a residue found at the same or corresponding position in another Cys10 protein (see FIGS. 1A and 1B), more preferably to a residue found at the same or corresponding position in an NsG31 sequence from another species, such as the species shown in FIG. 4. In a preferred embodiment, less than 8 amino acids have been changed, more preferably less than 5 amino acids, more preferably 1 or 2 amino acids, more preferably no amino acids have been changed. These truncated NsG31 polypeptides may have up to 5 additional C and/or N-terminal amino acids selected from those of the mature NsG31 polypeptides, i.e. fragments up to AA₁₀-AA₉₅ of SEQ ID No 4 and fragments up to AA₁₀-AA₉₅ of SEQ ID No. 10.

Further specific fragment are selected from the group consisting of SEQ ID No 6 and 12, and sequence variants of said polypeptides, wherein any amino acid specified in the chosen sequence is changed to a different amino acid, provided that no more than 10 of the amino acid residues in the sequence are so changed. These isolated polypeptides constitute the NsG31 core sequence, which is conserved in the Cys10 subfamily shown in FIG. 1A. Preferably any changes in these peptides are to residues marked in the alignment of FIG. 1A as unconserved, weakly conserved, or strongly conserved. More preferably any changed amino acids are selected from those designated as unconserved, weakly conserved or strongly conserved in FIG. 4. Preferably any changed amino acid is changed to a residue found at the same or corresponding position in another Cys10 protein (see FIGS. 1A and 1B), more preferably to a residue found at the same or corresponding position in an NsG31 sequence from another species, such as the species shown in FIG. 4. In a preferred embodiment, less than 8 amino acids have been changed, more preferably less than 5 amino acids, more preferably 1 or 2 amino acids, more preferably no amino acids have been changed. These truncated NsG31 polypeptides may have up to 5 additional C and/or N-terminal amino acids selected from those of the mature NsG31 polypeptides, i.e. fragments up to AA₈-AA₁₀₃ of SEQ ID No 4, and fragments up to AA₈-AA₁₀₃ of SEQ ID No. 10.

Further fragments include those consisting of AA₁₄-AA₁₃₉ of SEQ ID No. 16 and AA₁₆-AA₁₃₉ of SEQ ID No. 16 and variants of these fragments, wherein any amino acid specified in the chosen sequence is changed to a different amino acid, provided that no more than 15 of the amino acid residues in the sequence are so changed. These fragments are N-terminally truncated polypeptide fragment of NsG31-long truncated down to the N-terminal of the Cys10 subfamily (FIG. 1A) core sequence (AA₁₄) and to the first conserved cysteine (AA₁₆) respectively.

Variants within the scope of the invention in one embodiment include proteins and peptides with amino acid sequences having at least 60 percent identity with human or mouse NsG31 (SEQ ID NO: 3 and 9). More preferably the sequence identity is at least 65%, more preferably at least 70%, more preferably at least 75%, more preferably at least 80%, more preferably at least 85%, more preferably at least 90%, more preferably at least 95%, more preferably at least 98%.

Variants within the scope of the invention in one embodiment include proteins and peptides with amino acid sequences having at least 60 percent identity with a polypeptide having a sequence selected from the group consisting of SEQ ID NO: 3, 4, 5, 6, 9, 10, 11, and 12. More preferably the sequence identity is at least 65%, more preferably at least 70%, more preferably at least 75%, more preferably at least 80%, more preferably at least 85%, more preferably at least 90%, more preferably at least 95%, more preferably at least 98%. These polypeptides constitute full length NsG31, mature NsG31, and fragments, which are similar to the other members of the Cys10 family.

Variants within the scope of the invention in one embodiment include proteins and peptides with amino acid sequences having at least 60 percent identity with a polypeptide having the sequence of SEQ ID NO: 3, 4, 5, 6, 15, and 16. More preferably the sequence identity is at least 65%, more preferably at least 70%, more preferably at least 75%, more preferably at least 80%, more preferably at least 85%, more preferably at least 90%, more preferably at least 95%, more preferably at least 98%. These polypeptides constitute full length human NsG31, mature human NsG31 and fragments thereof.

Variants within the scope of the invention in one embodiment include proteins and peptides with amino acid sequences having at least 60 percent identity with a polypeptide having the sequence of SEQ ID NO: 4 and 10, More preferably the sequence identity is at least 65%, more preferably at least 70%, more preferably at least 75%, more preferably at least 80%, more preferably at least 85%, more preferably at least 90%, more preferably at least 95%, more preferably at least 98%.

Variants within the scope of the invention in one embodiment include proteins and peptides with amino acid sequences having at least 60 percent identity with a polypeptide having the sequence of SEQ ID NO: 5 and 11, More preferably the sequence Identity is at least 65%, more preferably at least 70%, more preferably at least 75%, more preferably at least 80%, more preferably at least 85%, more preferably at least 90%, more preferably at least 95%, more preferably at least 98%.

Variants within the scope of the invention in one embodiment include proteins and peptides with amino acid sequences having at least 60 percent identity with a polypeptide having the sequence of SEQ ID NO: 6 and 12, More preferably the sequence identity is at least 65%, more preferably at least 70%, more preferably at least 75%, more preferably at least 80%, more preferably at least 85%, more preferably at least 90%, more preferably at least 95%, more preferably at least 98%.

For the purposes of determining homology the minimum length of comparison sequences will generally be at least 8 amino acid residues, usually at least 12 amino acid residues. For the purposes of the present invention, the percent sequence identity is preferably calculated in a range of overlap of at least 25 amino acids, more preferably at least 30 amino acids, more preferably at least 35, more preferably at least 40, more preferably at least 45, more preferably at least 50, more preferably at least 55, more preferably at least 60, such as at least 70, for example at least 80, such as at least 90, the range being determined by BLASTP under default settings.

In one embodiment the percent sequence identity is calculated using global alignment (GAP or Align), so that the variant and SEQ ID sequences are aligned, the total number of identical amino acid residues calculated and divided by the length of the SEQ ID NO.

In one embodiment, a variant NsG31 comprises a naturally occurring allelic variant of the sequence selected from the group consisting of SEQ ID No 3, 4, 5, 6, 9, 10, 11, 12, 15, and 16. Said allelic variant sequence may be an amino acid sequence that is the translation of a nucleic acid sequence differing by a single nucleotide from a nucleic acid sequence selected from the group consisting of SEQ ID No 1, 2, 7, 8, 13 and 14.

In one embodiment, the variants include proteins comprising an amino acid sequence having at least 60% sequence identity to SEQ ID NO 3, more preferably at least 65%, more preferably at least 70%, more preferably at least 75%, more preferably at least 80%, more preferably at least 85%, more preferably at least 90%, more preferably at least 95%, more preferably at least 98%.

In one embodiment, the variants include proteins comprising an amino acid sequence having at least 60% sequence identity to SEQ ID NO 4, more preferably at least 65%, more preferably at least 70%, more preferably at least 75%, more preferably at least 80%, more preferably at least 85%, more preferably at least 90%, more preferably at least 95%, more preferably at least 98%.

In one embodiment, the variants include proteins comprising an amino acid sequence having at least 60% sequence identity to SEQ ID NO 5, more preferably at least 65%, more preferably at least 70%, more preferably at least 75%, more preferably at least 80%, more preferably at least 85%, more preferably at least 90%, more preferably at least 95%, more preferably at least 98%.

In one embodiment, the variants include proteins comprising an amino acid sequence having at least 60% sequence Identity to SEQ ID NO 6, more preferably at least 65%, more preferably at least 70%, more preferably at least 75%, more preferably at least 80%, more preferably at least 85%, more preferably at least 90%, more preferably at least 95%, more preferably at least 98%.

In one embodiment, the variants include proteins comprising an amino acid sequence having at least 60% sequence identity to SEQ ID NO 9, more preferably at least 65%, more preferably at least 70%, more preferably at least 75%, more preferably at least 80%, more preferably at least 85%, more preferably at least 90%, more preferably at least 95%, more preferably at least 98%.

In one embodiment, the variants include proteins comprising an amino acid sequence having at least 60% sequence identity to SEQ ID NO 10, more preferably at least 65%, more preferably at least 70%, more preferably at least 75%, more preferably at least 80%, more preferably at least 85%, more preferably at least 90%, more preferably at least 95%, more preferably at least 98%.

In one embodiment, the variants include proteins comprising an amino acid sequence having at least 60% sequence identity to SEQ ID NO 11, more preferably at least 65%, more preferably at least 70%, more preferably at least 75%, more preferably at least 80%, more preferably at least 85%, more preferably at least 90%, more preferably at least 95%, more preferably at least 98%.

In one embodiment, the variants include proteins comprising an amino acid sequence having at least 60% sequence identity to SEQ ID NO 12, more preferably at least 65%, more preferably at least 70%, more preferably at least 75%, more preferably at least 80%, more preferably at least 85%, more preferably at least 90%, more preferably at least 95%, more preferably at least 98%.

In one embodiment, the variants include proteins comprising an amino acid sequence having at least 60% sequence identity to SEQ ID NO 15, more preferably at least 65%, more preferably at least 70%, more preferably at least 75%, more preferably at least 80%, more preferably at least 85%, more preferably at least 90%, more preferably at least 95%, more preferably at least 98%.

In one embodiment, the variants include proteins comprising an amino acid sequence having at least 60% sequence identity to SEQ ID NO 16, more preferably at least 65%, more preferably at least 70%, more preferably at least 75%, more preferably at least 80%, more preferably at least 85%, more preferably at least 90%, more preferably at least 95%, more preferably at least 98%.

In one embodiment, a variant NsG31 at corresponding positions comprises the residues marked in FIG. 4 as fully conserved (*), more preferably a variant NsG31 also comprises at corresponding positions the residues marked in FIG. 4 as strongly conserved (: strongly conserved groups include: STA, NEQK, NHQK, NEDQ, QHRK, MILV, MILF, HY FYW), more preferably a variant NsG31 also comprises at corresponding positions the residues marked in FIG. 4 as less conserved (. less conserved groups include: CSA, ATV, SAG, STNK, STPA, SGND, SNDEQK, NDEQHK, NEQHK, NEQHRK, VLIM, HFY). In particular, it is contemplated that the conserved cysteines (FIG. 1B) preferably are located at corresponding positions in a variant NsG31.

### Reference is Now Made to NsG29 Polypeptides

A preferred biological activity is the ability to elicit substantially the same response as in the PC12 assay described in the Examples. In this assay PC12 cells are transduced with a sequence coding for full length human NsG29 (Example 3b). By substantially the same response in the PC12 assay is intended that the number of neurite bearing cells is at least 10% of the number obtained in Example 5 (transduction with full length human NsG29), more preferably at least 20%, more preferably at least 30%, more preferably at least 40%, more preferably at least 50%, more preferably at least 60%, more preferably at least 70%, more preferably at least 75%, more preferably at least 80%, more preferably at least 85%, more preferably at least 90%. The PC12 assay may also be used to document the percentage improvement in survival over a control treatment. Substantially the same response in this context means an activity resulting in at least 10% of the improvement obtained in Example 5, more preferably at least 20%, more preferably at least 30%, more preferably at least 40%, more preferably at least 50%, more preferably at least 60%, more preferably at least 70%, more preferably at least 75%, more preferably at least 80%, more preferably at least 90%. The biological activity of a fragment or variant of NsG29 may also be higher than that of the naturally occurring NsG29.

Specific fragments of NsG29 include polypeptides having a sequence of SEQ ID No. 20, and sequence variants of said polypeptides, wherein any amino acid specified in the chosen sequence is changed to a different amino acid, provided that no more than 10 of the amino acid residues in the sequence are so changed. These isolated polypeptides constitute NsG29 polypeptide spanning from the first to the last of the 10 conserved cysteine residues of the Cys10 family. It is believed that the biological activity mainly resides in this part of the protein, and that the sequence with all the ten cysteines is required for correct folding of the protein. Preferably any changes in these peptides are to residues marked in the alignment of FIG. 1A as unconserved, weakly conserved, or strongly conserved. Preferably any changed amino acid is changed to a residue found at the same or corresponding position in another Cys10 protein (see FIGS. 1A and 1B). In a preferred embodiment, less than 8 amino acids have been changed, more preferably less than 5 amino acids, more preferably 1 or 2 amino acids, more preferably no amino acids have been changed. These truncated NsG29 polypeptides may have up to 5 additional C and/or N-terminal amino acids selected from those of the mature NsG29 polypeptides, i.e. fragments up to AA₁₄-AA₉₈ of SEQ ID No 19.

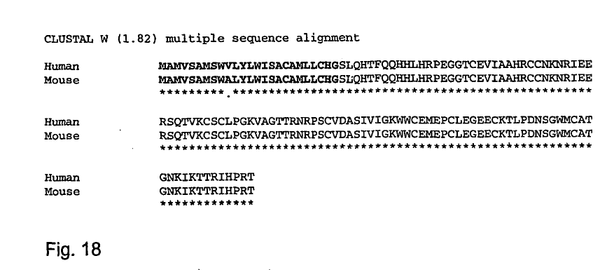

Further specific polypeptides have the sequence of SEQ ID No 21, and sequence variants of said polypeptide, wherein any amino acid specified in the chosen sequence is changed to a different amino acid, provided that no more than 10 of the amino acid residues in the sequence are so changed. These isolated polypeptides constitute the NsG29 core sequence, which is conserved in the Cys10 subfamily shown in FIG. 1A. Preferably any changes in these peptides are to residues marked in the alignment of FIG. 1A as unconserved, weakly conserved, or strongly conserved. Preferably any changed amino acid is changed to a residue found at the same or corresponding position in another Cys10 protein (see FIGS. 1A and 1B), more preferably to a residue found at the same or corresponding position in an NsG29 sequence from another species, such as the species shown in FIG. 18. In a preferred embodiment, less than 8 amino acids have been changed, more preferably less than 5 amino acids, more preferably 1 or 2 amino acids, more preferably no amino acids have been changed. These truncated NsG29 polypeptides may have up to 5 additional C and/or N-terminal amino acids selected from those of the mature NsG29 polypeptides, i.e. fragments up to AA₁₂-AA₁₀₈ of SEQ ID No 19.

Variants within the scope of the invention in one embodiment include proteins and peptides with amino acid sequences having at least 60 percent identity with human or mouse NsG29 (SEQ ID NO: 18, and 23). More preferably the sequence identity is at least 65%, more preferably at least 70%, more preferably at least 75%, more preferably at least 80%, more preferably at least 85%, more preferably at least 90%, more preferably at least 95%, more preferably at least 98%.

Variants within the scope of the invention in one embodiment include proteins and peptides with amino acid sequences having at least 60 percent identity with a polypeptide having a sequence selected from the group consisting of SEQ ID NO: 18, 19, 20, and 21. More preferably the sequence identity is at least 65%, more preferably at least 70%, more preferably at least 75%, more preferably at least 80%, more preferably at least 85%, more preferably at least 90%, more preferably at least 95%, more preferably at least 98%.

In one embodiment, a variant NsG29 comprises a naturally occurring allelic variant of the sequence selected from the group consisting of SEQ ID No 18, 19, 20, 21, and 23. Said allelic variant sequence may be an amino acid sequence that is the translation of a nucleic acid sequence differing by a single nucleotide from a nucleic acid sequence selected from the group consisting of SEQ ID No 17 and 22.

In one embodiment, the variants include proteins comprising an amino acid sequence having at least 60% sequence identity to SEQ ID NO 18, more preferably at least 65%, more preferably at least 70%, more preferably at least 75%, more preferably at least 80%, more preferably at least 85%, more preferably at least 90%, more preferably at least 95%, more preferably at least 98%.

In one embodiment, the variants include proteins comprising an amino acid sequence having at least 60% sequence identity to SEQ ID NO 19, more preferably at least 65%, more preferably at least 70%, more preferably at least 75%, more preferably at least 80%, more preferably at least 85%, more preferably at least 90%, more preferably at least 95%, more preferably at least 98%.

In one embodiment, the variants include proteins comprising an amino acid sequence having at least 60% sequence identity to SEQ ID NO 20, more preferably at least 65%, more preferably at least 70%, more preferably at least 75%, more preferably at least 80%, more preferably at least 85%, more preferably at least 90%, more preferably at least 95%, more preferably at least 98%.

In one embodiment, the variants include proteins comprising an amino acid sequence having at least 60% sequence identity to SEQ ID NO 21, more preferably at least 65%, more preferably at least 70%, more preferably at least 75%, more preferably at least 80%, more preferably at least 85%, more preferably at least 90%, more preferably at least 95%, more preferably at least 98%.

In one embodiment, the variants include proteins comprising an amino acid sequence having at least 60% sequence identity to SEQ ID NO 23, more preferably at least 65%, more preferably at least 70%, more preferably at least 75%, more preferably at least 80%, more preferably at least 85%, more preferably at least 90%, more preferably at least 95%, more preferably at least 98%.

In one embodiment, a variant NsG29 at corresponding positions comprises the residues marked in FIG. 18 as fully conserved (*), more preferably a variant NsG29 also comprises at corresponding positions the residues marked in FIG. 18 as strongly conserved (: strongly conserved groups include: STA, NEQK, NHQK, NEDQ, QHRK, MILV, MILF, HY FYW), more preferably a variant NsG29 also comprises at corresponding positions the residues marked in FIG. 18 as less conserved (. less conserved groups include: CSA, ATV, SAG, STNK, STPA, SGND, SNDEQK, NDEQHK, NEQHK, NEQHRK, VLIM, HFY). In particular, it is contemplated that the conserved cysteines (FIG. 1A) preferably are located at corresponding positions in a variant NsG29.

One specific variant is a possible SNP leading to W instead of G at position 92 in human full length NsG29 (SEQ ID No. 18) or the corresponding position in SEQ ID No. 19, 20, 21 or a fragment of NsG29.

### IV NSG29 and NSG31 Nucleotide Sequences

The invention in one aspect provides medical use of genomic DNA and cDNA coding for NsG31, including for example the human genomic nucleotide sequence (SEQ ID No. 1 and 13), mouse genomic nucleotide sequence (SEQ ID No. 7), the nucleotide sequence of human, and mouse NsG31 cDNA (SEQ ID NO 2, and 8), and the nucleotide sequence of human NsG31-long cDNA (SEQ ID No. 14).

Variants of these sequences are also included within the scope of the present invention.

The invention relates to an isolated nucleic acid molecule for medical use comprising a nucleotide sequence selected from the group consisting of:

a) a nucleotide sequence coding for a polypeptide having the amino acid sequence selected from the group consisting of SEQ ID No. 3, 4, 5, 6, 9, 10, 11, 12, 15, and 16;
b) a nucleotide sequence coding for a sequence variant of the amino acid sequence selected from the group consisting of SEQ ID No. 3, 4, 5, 6, 9, 10, 11, 12, 15, and 16, wherein the variant has at least 70% sequence identity to said SEQ ID No.;
c) a nucleotide sequence coding for a biologically active fragment of at least 50 contiguous amino acids of any of a) through b);
d) a nucleotide sequence selected from the group consisting of SEQ ID No. 1, 2, 7, 8, 13, and 14;
e) a nucleotide sequence having at least 70% sequence identity to a coding sequence selected from the group consisting of the coding sequence of SEQ ID No. 2, 8, or 14;
f) a nucleic acid sequence of at least 150 contiguous nucleotides of a coding sequence selected from the group consisting of the coding sequence of SEQ ID No. 2, 8, and 14;
g) the complement of a nucleic acid capable of hybridising with a nucleic acid molecule having the sequence of the coding sequence of SEQ ID No.: 2, 8, and 14 under conditions of high stringency; and h) the nucleic acid sequence of the complement of any of the above.

The nucleic acid molecule may comprise the nucleotide sequence of a naturally occurring allelic nucleic acid variant.

The nucleic acid molecule of the invention may encode a variant polypeptide, wherein the variant polypeptide has the polypeptide sequence of a naturally occurring polypeptide variant.

In one embodiment the nucleic acid molecule differs by a single nucleotide from a nucleic acid sequence selected from the group consisting of SEQ ID No. 1, 2, 7, 8, 13, and 14, in particular 2, 8, and 14.

Preferably the encoded polypeptide has at least 60% sequence identity to a sequence selected from the group consisting of SEQ ID No. 3, 4, 5, 6, 15, and 16 preferably at least 65% sequence identity, more preferably at least 70% sequence identity, more preferably, 75% sequence identity, more preferably at least 80% sequence identity, more preferably at least 85% sequence Identity, more preferably at least 90% sequence identity, more preferably at least 95% sequence identity, more preferably at least 98% sequence identity, more preferably wherein the polypeptide has a sequence selected from the group consisting of said SEQ ID No.s. Said sequences constitute human NsG31.

In a preferred embodiment the encoded polypeptide has at least 60% sequence identity to a sequence selected from the group consisting of SEQ ID No. 3, 4, 5, 6, 9, 10, 11, and 12, preferably at least 65% sequence identity, more preferably at least 70% sequence identity, more preferably, 75% sequence Identity, more preferably at least 80% sequence identity, more preferably at least 85% sequence identity, more preferably at least 90% sequence identity, more preferably at least 95% sequence identity, more preferably at least 98% sequence identity, more preferably wherein the polypeptide has a sequence selected from the group consisting of said SEQ ID No.s.

In a preferred embodiment the encoded polypeptide has at least 60% sequence identity to a sequence selected from the group consisting of SEQ ID No. 3 and 9, preferably at least 65% sequence identity, more preferably at least 70% sequence identity, more preferably, 75% sequence identity, more preferably at least 80% sequence identity, more preferably at least 85% sequence identity, more preferably at least 90% sequence identity, more preferably at least 95% sequence identity, more preferably at least 98% sequence identity, more preferably wherein the polypeptide has a sequence selected from the group consisting of said SEQ ID No.s.

In a preferred embodiment the encoded polypeptide has at least 60% sequence identity to a sequence selected from the group consisting of SEQ ID No. 4 and 10, preferably at least 65% sequence identity, more preferably at least 70% sequence Identity, more preferably, 75% sequence Identity, more preferably at least 80% sequence identity, more preferably at least 85% sequence identity, more preferably at least 90% sequence identity, more preferably at least 95% sequence identity, more preferably at least 98% sequence identity, more preferably wherein the polypeptide has a sequence selected from the group consisting of said SEQ ID No.s.

In a preferred embodiment the encoded polypeptide has at least 60% sequence identity to a sequence selected from the group consisting of SEQ ID No. 5 and 11, preferably at least 65% sequence identity, more preferably at least 70% sequence identity, more preferably, 75% sequence identity, more preferably at least 80% sequence identity, more preferably at least 85% sequence identity, more preferably at least 90% sequence Identity, more preferably at least 95% sequence identity, more preferably at least 98% sequence identity, more preferably wherein the polypeptide has a sequence selected from the group consisting of said SEQ ID No.s.

In a preferred embodiment the encoded polypeptide has at least 60% sequence identity to a sequence selected from the group consisting of SEQ ID No. 6 and 12, preferably at least 65% sequence identity, more preferably at least 70% sequence Identity, more preferably, 75% sequence identity, more preferably at least 80% sequence Identity, more preferably at least 85% sequence identity, more preferably at least 90% sequence identity, more preferably at least 95% sequence identity, more preferably at least 98% sequence identity, more preferably wherein the polypeptide has a sequence selected from the group consisting of said SEQ ID No.s.

In a preferred embodiment the encoded polypeptide has at least 60% sequence identity to the sequence of SEQ ID No. 3, preferably at least 65% sequence identity, more preferably at least 70% sequence identity, more preferably, 75% sequence identity, more preferably at least 80% sequence identity, more preferably at least 85% sequence identity, more preferably at least 90% sequence identity, more preferably at least 95% sequence identity, more preferably at least 98% sequence identity, more preferably wherein the polypeptide has a sequence selected from the group consisting of said SEQ ID No.

In a preferred embodiment the encoded polypeptide has at least 60% sequence identity to the sequence of SEQ ID No. 4, preferably at least 65% sequence Identity, more preferably at least 70% sequence identity, more preferably, 75% sequence identity, more preferably at least 80% sequence identity, more preferably at least 85% sequence identity, more preferably at least 90% sequence identity, more preferably at least 95% sequence identity, more preferably at least 98% sequence identity, more preferably wherein the polypeptide has a sequence selected from the group consisting of said SEQ ID No.

In a preferred embodiment the encoded polypeptide has at least 60% sequence identity to the sequence of SEQ ID No. 5, preferably at least 65% sequence identity, more preferably at least 70% sequence identity, more preferably, 75% sequence identity, more preferably at least 80% sequence identity, more preferably at least 85% sequence identity, more preferably at least 90% sequence identity, more preferably at least 95% sequence identity, more preferably at least 98% sequence identity, more preferably wherein the polypeptide has a sequence selected from the group consisting of said SEQ ID No.

In a preferred embodiment the encoded polypeptide has at least 60% sequence identity to the sequence of SEQ ID No. 6, preferably at least 65% sequence identity, more preferably at least 70% sequence identity, more preferably, 75% sequence identity, more preferably at least 80% sequence identity, more preferably at least 85% sequence identity, more preferably at least 90% sequence identity, more preferably at least 95% sequence identity, more preferably at least 98% sequence identity, more preferably wherein the polypeptide has a sequence selected from the group consisting of said SEQ ID No.

In a preferred embodiment the encoded polypeptide has at least 60% sequence identity to the sequence of SEQ ID No. 15, preferably at least 65% sequence identity, more preferably at least 70% sequence identity, more preferably, 75% sequence identity, more preferably at least 80% sequence identity, more preferably at least 85% sequence identity, more preferably at least 90% sequence identity, more preferably at least 95% sequence identity, more preferably at least 98% sequence identity, more preferably wherein the polypeptide has a sequence selected from the group consisting of said SEQ ID No.

In a preferred embodiment the encoded polypeptide has at least 60% sequence identity to the sequence of SEQ ID No. 16, preferably at least 65% sequence identity, more preferably at least 70% sequence identity, more preferably, 75% sequence identity, more preferably at least 80% sequence identity, more preferably at least 85% sequence identity, more preferably at least 90% sequence identity, more preferably at least 95% sequence identity, more preferably at least 98% sequence identity, more preferably wherein the polypeptide has a sequence selected from the group consisting of said SEQ ID No.

In one aspect the nucleic acid molecule comprises a nucleotide sequence selected from the group consisting of

a) the nucleotide sequence selected from the group consisting of SEQ ID No. 1, 2, 7, 8, 13, and 14;
b) a nucleotide sequence having at least 70% sequence identity to a coding sequence selected from the group consisting of the coding sequence of SEQ ID No. 2, 8, and 14;
c) a nucleic acid sequence of at least 150 contiguous nucleotides of a coding sequence selected from the group consisting of the coding sequence of SEQ ID No. 2, 8 and 14;
d) the complement of a nucleic acid capable of hybridising with a nucleic acid having a sequence selected from the group consisting of the coding sequence of SEQ ID No.: 2, 8, and 14 under conditions of high stringency; and
e) the nucleic acid sequence of the complement of any of the above.

SEQ ID No 4, 5, 6, 10, 11, 12, and 15 represent the polypeptide sequences of mature NsG31 and NsG31 fragments. For recombinant expression in a eukaryotic expression system, these are preferably ligated to an appropriate signal sequence to ensure that the NsG31 polypeptide is secreted from the cells.

In one preferred embodiment, the isolated polynucleotide of the invention has at least 50%, preferably at least 60%, more preferably at least 70%, more preferably at least 75%, more preferably at least 80%, preferably at least 85%, more preferred at least 90%, more preferred at least 95%, more preferred at least 98% sequence identity to a polynucleotide sequence selected from the group consisting of the coding sequence of SEQ ID NO: 2, 8, and 14.

In one preferred embodiment, the isolated polynucleotide of the invention has at least 50%, preferably at least 60%, more preferably at least 70%, more preferably at least 75%, more preferably at least 80%, preferably at least 85%, more preferred at least 90%, more preferred at least 95%, more preferred at least 98% sequence identity to a polynucleotide sequence selected from the group consisting of the coding sequence of SEQ ID NO: 2 and 8.

In one embodiment, the isolated polynucleotide of the invention has at least 60, more preferably at least 65%, more preferably at least 70%, more preferably at least 75%, more preferably at least 80%, preferably at least 85%, more preferred at least 90%, more preferred at least 95%, more preferred at least 98% sequence identity to the polynucleotide sequence presented as SEQ ID NO: 1.

In one preferred embodiment, the isolated polynucleotide of the invention has at least 50%, preferably at least 60%, more preferably at least 70%, more preferably at least 75%, more preferably at least 80%, preferably at least 85%, more preferred at least 90%, more preferred at least 95%, more preferred at least 98% sequence identity to the coding sequence of SEQ ID NO: 2.

In one embodiment, the Isolated polynucleotide of the invention has at least 50%, preferably at least 60%, more preferably at least 70%, more preferably at least 75%, more preferably at least 80%, preferably at least 85%, more preferred at least 90%, more preferred at least 95%, more preferred at least 98% sequence identity to a polynucleotide sequence presented as SEQ ID NO: 7.

In one preferred embodiment, the isolated polynucleotide of the invention has at least 50%, preferably at least 60%, more preferably at least 70%, more preferably at least 75%, more preferably at least 80%, preferably at least 85%, more preferred at least 90%, more preferred at least 95%, more preferred at least 98% sequence identity to the coding sequence of SEQ ID NO: 8.

In one embodiment, the isolated polynucleotide of the invention has at least 50%, preferably at least 60%, more preferably at least 70%, more preferably at least 75%, more preferably at least 80%, preferably at least 85%, more preferred at least 90%, more preferred at least 95%, more preferred at least 98% sequence identity to a polynucleotide sequence presented as SEQ ID NO: 13.

In one preferred embodiment, the isolated polynucleotide of the invention has at least 50%, preferably at least 60%, more preferably at least 70%, more preferably at least 75%, more preferably at least 80%, preferably at least 85%, more preferred at least 90%, more preferred at least 95%, more preferred at least 98% sequence identity to the coding sequence of SEQ ID NO: 14.

A preferred isolated polynucleotide includes SEQ ID No 1 and 2, which are human NsG31 polynucleotides. Another preferred group of isolated polynucleotides include SEQ ID No. 2 and 8, more preferably the coding sequence of SEQ ID No. 2 and 8, which represent the cDNA sequences. Generally the cDNA sequences are much shorter than the genomic sequences and are more easily inserted into an appropriate expression vector for transduction/transfection into a production cell or a human cell in vivo or ex vivo. The coding sequences are even shorter but may require addition of appropriate 3′ and 5′ untranslated sequences for correct expression.

The nucleotide sequence of nucleic acids coding for full length NsG31, for mature NsG31 and for fragments of NsG31 can be derived from FIGS. 10, 11, and 12, which show the cDNA sequence and the translated peptides of human NsG31 (FIG. 10), mouse NsG31 (FIG. 11), and human NsG31-long (FIG. 12). Specific fragments of these cDNA sequences include those coding for mature NsG31, mature NsG31-long and fragments of NsG31 and NsG31 long, including the nucleic acid molecule having the nucleotide sequence of nucleotides 70471 of SEQ ID No. 2 (human NsG31 CDS); the nucleic acid molecule having the nucleotide sequence of nucleotides 160-468 of SEQ ID No. 2 (human mature NsG31); the nucleic acid molecule having the nucleotide sequence of nucleotides 205-429 of SEQ ID No. 2 (human Cys1-Cys10 fragment); the nucleic acid molecule having the nucleotide sequence of nucleotides 199-459 of SEQ ID No. 2 (human core fragment); the nucleic acid molecule having the nucleotide sequence of nucleotides 192-590 of SEQ ID No. 8 (mouse NsG31 CDS); the nucleic acid molecule having the nucleotide sequence of nucleotides 279-587 of SEQ ID No. 8 (mouse mature NsG31); the nucleic acid molecule having the nucleotide sequence of nucleotides 324-548 of SEQ ID No. 8 (mouse Cys1-Cys10 fragment); the nucleic acid molecule having the nucleotide sequence of nucleotides 318-578 of SEQ ID No. 8 (mouse core fragment); the nucleic acid molecule having the nucleotide sequence of nucleotides 70-579 of SEQ ID No. 14 (human NsG31-long CDS); the nucleic acid molecule having the nucleotide sequence of nucleotides 160-576 of SEQ ID No. 14 (mature human NsG31-long); and the nucleic acid molecule having the nucleotide sequence of nucleotides 199-576 of SEQ ID No. 14 (N-truncated human NsG31-long).

In the aspects relating to NsG29, the invention provides medical use of genomic DNA and cDNA coding for NsG29, including for example the nucleotide sequence of human, and mouse NsG29 cDNA (SEQ ID NO 17 and 22).

Variants of these sequences are also included within the scope of the present invention.

The invention relates to an isolated nucleic acid molecule for medical use comprising a nucleotide sequence selected from the group consisting of:

a) a nucleotide sequence coding for a polypeptide having the amino acid sequence selected from the group consisting of SEQ ID No. 18, 19, 20, 21, and 23;
b) a nucleotide sequence coding for a sequence variant of the amino acid sequence selected from the group consisting of SEQ ID No. 18, 19, 20, 21, and 21, wherein the variant has at least 70% sequence identity to said SEQ ID No.;
c) a nucleotide sequence coding for a biologically active fragment of at least 50 contiguous amino acids of any of a) through b);
d) a nucleotide sequence selected from the group consisting of SEQ ID No. 17 and 22;
e) a nucleotide sequence having at least 70% sequence identity to a coding sequence selected from the group consisting of the coding sequence of SEQ ID No. 17 and 22;
f) a nucleic acid sequence of at least 150 contiguous nucleotides of a coding sequence selected from the group consisting of the coding sequence of SEQ ID No. 17 and 22;
g) the complement of a nucleic acid capable of hybridising with a nucleic acid molecule having the sequence of the coding sequence of SEQ ID No.: 17 and 22 under conditions of high stringency;
h) the nucleic acid sequence of the complement of any of the above.

The nucleic acid molecule may comprise the nucleotide sequence of a naturally occurring allelic nucleic acid variant.

The nucleic acid molecule of the invention may encode a variant polypeptide, wherein the variant polypeptide has the polypeptide sequence of a naturally occurring polypeptide variant.

In one embodiment the nucleic acid molecule differs by a single nucleotide from a nucleic acid sequence selected from the group consisting of SEQ ID No. 17 and 22.

Preferably the encoded polypeptide has at least 60% sequence identity to a sequence selected from the group consisting of SEQ ID No. 18, 19, 20, and 21 preferably at least 65% sequence identity, more preferably at least 70% sequence identity, more preferably, 75% sequence identity, more preferably at least 80% sequence identity, more preferably at least 85% sequence identity, more preferably at least 90% sequence identity, more preferably at least 95% sequence identity, more preferably at least 98% sequence identity, more preferably wherein the polypeptide has a sequence selected from the group consisting of said SEQ ID No.s. Said sequences constitute human NsG29.

In a preferred embodiment the encoded polypeptide has at least 60% sequence identity to a sequence selected from the group consisting of SEQ ID No. 18 and 23, preferably at least 65% sequence identity, more preferably at least 70% sequence identity, more preferably, 75% sequence identity, more preferably at least 80% sequence identity, more preferably at least 85% sequence identity, more preferably at least 90% sequence identity, more preferably at least 95% sequence identity, more preferably at least 98% sequence identity, more preferably wherein the polypeptide has a sequence selected from the group consisting of said SEQ ID No.s.

In a preferred embodiment the encoded polypeptide has at least 60% sequence Identity to the sequence of SEQ ID No. 18, preferably at least 65% sequence identity, more preferably at least 70% sequence identity, more preferably, 75% sequence identity, more preferably at least 80% sequence identity, more preferably at least 85% sequence identity, more preferably at least 90% sequence identity, more preferably at least 95% sequence identity, more preferably at least 98% sequence identity, more preferably wherein the polypeptide has a sequence selected from the group consisting of said SEQ ID No.

In a preferred embodiment the encoded polypeptide has at least 60% sequence identity to the sequence of SEQ ID No. 19, preferably at least 65% sequence identity, more preferably at least 70% sequence identity, more preferably, 75% sequence identity, more preferably at least 80% sequence identity, more preferably at least 85% sequence identity, more preferably at least 90% sequence identity, more preferably at least 95% sequence identity, more preferably at least 98% sequence identity, more preferably wherein the polypeptide has a sequence selected from the group consisting of said SEQ ID No.

In a preferred embodiment the encoded polypeptide has at least 60% sequence identity to the sequence of SEQ ID No. 20, preferably at least 65% sequence Identity, more preferably at least 70% sequence identity, more preferably, 75% sequence identity, more preferably at least 80% sequence identity, more preferably at least 85% sequence identity, more preferably at least 90% sequence identity, more preferably at least 95% sequence identity, more preferably at least 98% sequence identity, more preferably wherein the polypeptide has a sequence selected from the group consisting of said SEQ ID No.

In a preferred embodiment the encoded polypeptide has at least 60% sequence Identity to the sequence of SEQ ID No. 21, preferably at least 65% sequence identity, more preferably at least 70% sequence identity, more preferably, 75% sequence identity, more preferably at least 80% sequence identity, more preferably at least 85% sequence identity, more preferably at least 90% sequence identity, more preferably at least 95% sequence identity, more preferably at least 98% sequence identity, more preferably wherein the polypeptide has a sequence selected from the group consisting of said SEQ ID No.

In a preferred embodiment the encoded polypeptide has at least 60% sequence identity to the sequence of SEQ ID No. 23, preferably at least 65% sequence identity, more preferably at least 70% sequence identity, more preferably, 75% sequence identity, more preferably at least 80% sequence identity, more preferably at least 85% sequence identity, more preferably at least 90% sequence identity, more preferably at least 95% sequence identity, more preferably at least 98% sequence identity, more preferably wherein the polypeptide has a sequence selected from the group consisting of said SEQ ID No.

SEQ ID No 19, 20, and 21 represent the polypeptide sequences of NsG29 fragments. For recombinant expression in a eukaryotic expression system, these are preferably ligated to an appropriate signal sequence to ensure that the NsG29 polypeptide is secreted from the cells.

In one aspect the nucleic acid molecule comprises a nucleotide sequence selected from the group consisting of

a) the nucleotide sequence selected from the group consisting of SEQ ID No. 17 and 22;
b) a nucleotide sequence having at least 70% sequence Identity to a nucleotide sequence selected from the group consisting of the coding sequence of SEQ ID No. 17 and 22;
c) a nucleic acid sequence of at least 150 contiguous nucleotides of a sequence selected from the group consisting of the coding sequence of SEQ ID No. 17 and 22;
d) the complement of a nucleic acid capable of hybridising with nucleic acid having the sequence selected from the group consisting of the coding sequence of SEQ ID No.: 17 and 22 under conditions of high stringency; and
e) the nucleic acid sequence of the complement of any of the above.

In one preferred embodiment, the isolated polynucleotide of the invention has at least 50%, preferably at least 60%, more preferably at least 70%, more preferably at least 75%, more preferably at least 80%, preferably at least 85%, more preferred at least 90%, more preferred at least 95%, more preferred at least 98% sequence identity to a polynucleotide sequence selected from the group consisting of the coding sequence of SEQ ID NO: 17 and 22.

In one embodiment, the isolated polynucleotide of the invention has at least 60, more preferably at least 65%, more preferably at least 70%, more preferably at least 75%, more preferably at least 80%, preferably at least 85%, more preferred at least 90%, more preferred at least 95%, more preferred at least 98% sequence identity to the polynucleotide sequence presented as the coding sequence of SEQ ID NO: 17.

In one preferred embodiment, the isolated polynucleotide of the invention has at least 50%, preferably at least 60%, more preferably at least 70%, more preferably at least 75%, more preferably at least 80%, preferably at least 85%, more preferred at least 90%, more preferred at least 95%, more preferred at least 98% sequence identity to a polynucleotide sequence presented as the coding sequence of SEQ ID NO: 22.

A preferred isolated polynucleotide is SEQ ID No 17, which is a human NsG29 polynucleotide.

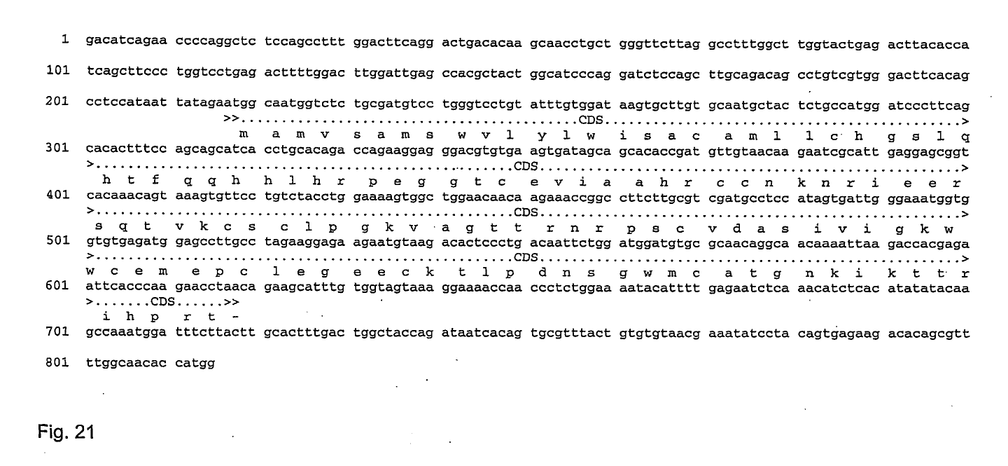

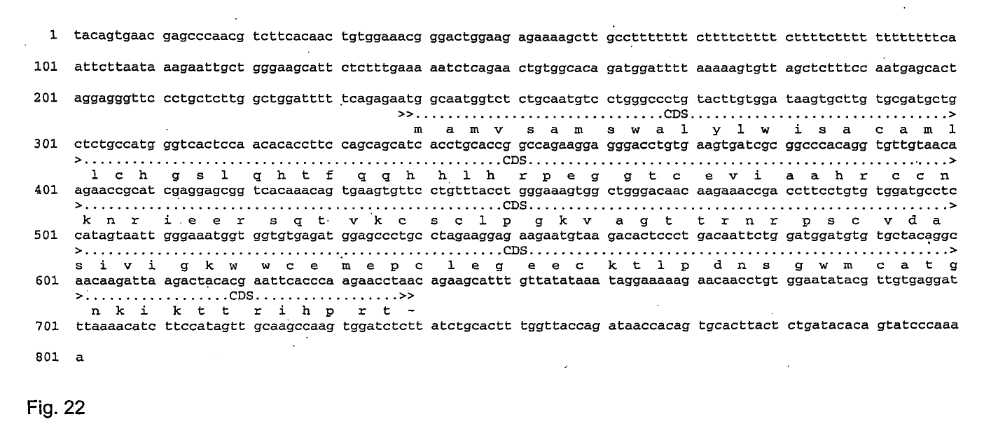

The nucleotide sequence of nucleic acids coding for full length NsG29, for mature NsG29 and for fragments of NsG29 can be derived from FIGS. 21 and 22, which show the cDNA sequence and the translated peptides of human NsG28 (FIG. 21) and mouse NsG28 (FIG. 22). Specific fragments of these cDNA sequences include those coding for mature NsG28, and fragments of NsG28, including the nucleic acid molecule having the nucleotide sequence of nucleotides 217-618 of SEQ ID No. 17 (human NsG29 CDS); the nucleic acid molecule having the nucleotide sequence of nucleotides 292-615 of SEQ ID No. 17 (human mature NsG29 CDS); the nucleic acid molecule having the nucleotide sequence of nucleotides 346-570 of SEQ ID No. 17 (human Cys1-Cys10 fragment CDS); the nucleic acid molecule having the nucleotide sequence of nucleotides 340-600 of SEQ ID No. 17 (human core fragment CDS); the nucleic acid molecule having the nucleotide sequence of nucleotides 238-639 of SEQ ID No. 22 (mouse NsG29 CDS); the nucleic acid molecule having the nucleotide sequence of nucleotides 313-636 of SEQ ID No. 22 (mouse mature NsG29 CDS); the nucleic acid molecule having the nucleotide sequence of nucleotides 367-591 of SEQ ID No. 22 (mouse Cys1-Cys10 fragment CDS); the nucleic acid molecule having the nucleotide sequence of nucleotides 361-621 of SEQ ID No. 22 (mouse core fragment CDS).

In addition, the nucleotide sequences of the invention include sequences, which are derivatives of these sequences. The invention also includes vectors, liposomes and other carrier vehicles, which encompass one of these sequences or a derivative of one of these sequences. The Invention also includes proteins transcribed and translated from NsG29 and/or NsG31 cDNA, preferably human NsG29 and/or NsG31 cDNA, including but not limited to human NsG29 and/or NsG31 and derivatives and variants.

In another embodiment, the invention relates to the use of the nucleic acids and proteins of the present invention to design probes to Isolate other genes, which encode proteins with structural or functional properties of the NsG29 and/or NsG31 proteins of the invention. The probes can be a variety of base pairs in length. For example, a nucleic acid probe can be between about 10 base pairs in length to about 150 base pairs in length.

Alternatively, the nucleic acid probe can be greater than about 150 base pairs in length. Experimental methods are provided in Ausubel et al., “Current Protocols in Molecular Biology”, J. Wiley (ed.) (1999), the entire teachings of which are herein incorporated by reference in their entirety.

The design of the oligonucleotide (also referred to herein as nucleic acid) probe should preferably follow these parameters:

i) it should be designed to an area of the sequence which has the fewest ambiguous bases, if any and
ii) it should be designed to have a calculated Tₘ of about 80° C. (assuming 2° C. for each A or T and 4° C. for each G or C).

The oligonucleotide should preferably be labelled to facilitate detection of hybridisation. Labelling may be with γ-³²p ATP (specific activity 6000 Ci/mmole) and T4 polynucleotide kinase using commonly employed techniques for labelling oligonucleotides. Other labeling techniques can also be used. Unincorporated label should preferably be removed by gel filtration chromatography or other established methods. The amount of radioactivity incorporated into the probe should be quantitated by measurement in a scintillation counter. Preferably, specific activity of the resulting probe should be approximately 4×10⁸ dpm/pmole. The bacterial culture containing the pool of full-length clones should preferably be thawed and 100 μL of the stock used to inoculate a sterile culture flask containing 25 ml of sterile L-broth containing ampicillin at 100 μg/ml.

The culture should preferably be grown to saturation at about 37° C., and the saturated culture should preferably be diluted in fresh L-broth. Aliquots of these dilutions should preferably be plated to determine the dilution and volume which will yield approximately 5000 distinct and well-separated colonies on solid bacteriological media containing L-broth containing ampicillin at 100 μg/ml and agar at 1.5% in a 150 mm petri dish when grown overnight at about 37° C. Other known methods of obtaining distinct, well-separated colonies can also be employed.

Standard colony hybridization procedures should then be used to transfer the colonies to nitrocellulose filters and lyse, denature and bake them. Highly stringent (also referred to herein as “high stringency”) conditions are those that are at least as stringent as, for example, 1×SSC at about 65° C., or 1×SSC and 50% formamide at about 42° C. “Moderate stringency” conditions are those that are at least as stringent as 4×SSC at about 65° C., or 4×SSC and 50% formamide at about 42° C. “Reduced stringency” conditions are those that are at least as stringent as 4×SSC at about 50° C., or 6×SSC and 50% formamide at 40° C.

The filter is then preferably incubated at about 65° C. for 1 hour with gentle agitation in 6×SSC (20× stock is 175.3 g NaCl/liter, 88.2 g Na citrate/liter, adjusted to pH 7.0 with NaOH) containing 0.5% SDS, 100 g/ml of yeast RNA, and 10 mM EDTA (approximately 10 mL per 150 mm filter). Preferably, the probe is then added to the hybridization mix at a concentration greater than or equal to 1×10⁶ dpm/mL. The filter is then preferably incubated at about 65° C. with gentle agitation overnight. The filter is then preferably washed in 500 mL of 2×SSC/0.5% SDS at room temperature without agitation, preferably followed by 500 mL of 2×SSC/0.1% SDS at room temperature with gentle shaking for 15 minutes. A third wash with 0.1×SSC/0.5% SDS at about 65° C. for 30 minutes to 1 hour is optional. The filter is then preferably dried and subjected to autoradiography for sufficient time to visualize the positives on the X-ray film. Other known hybridization methods can also be employed. The positive colonies are then picked, grown in culture, and plasmid DNA isolated using standard procedures. The clones can then be verified by restriction analysis, hybridisation analysis, or DNA sequencing.

Alternatively, suitable experimental conditions for determining hybridization between a nucleotide probe and a homologous DNA or RNA sequence, involves pre-soaking of the filter containing the DNA fragments or RNA to hybridize in 5×SSC [Sodium chloride/Sodium citrate; cf. Sambrook et al.; Molecular Cloning: A Laboratory Manual. Cold Spring Harbor Lab., Cold Spring Harbor, N.Y. 1989] for 10 minutes, and pre-hybridization of the filter in a solution of 5×SSC, 5×Denhardt's solution [cf. Sambrook et al.; Op cit.], 0.5% SDS and 100 μg/ml of denatured sonicated salmon sperm DNA [cf. Sambrook et al.; Op cit.], followed by hybridization in the same solution containing a concentration of 10 ng/ml of a random-primed [Feinberg A P & Vogelstein B; Anal. Biochem. 1983 132 6-13], ³²P-dCTP-labeled (specific activity>1×10⁹ cpm/μg) probe for 12 hours at approximately 45° C. The filter is then washed twice for 30 minutes in 0.1×SSC, 0.5% SDS at a temperature of at least at least 60° C. (medium stringency conditions), preferably of at least 65° C. (medium/high stringency conditions), more preferred of at least 70° C. (high stringency conditions), and even more preferred of at least 75° C. (very high stringency conditions). Molecules to which the oligonucleotide probe hybridizes under these conditions may be detected using a x-ray film.

In yet another embodiment, the invention relates to nucleic acid sequences (e.g., DNA, RNA) that hybridise to nucleic acids of NsG31. In particular, nucleic acids which hybridise to a molecule having a sequence selected from the group consisting of the coding sequence of SEQ ID NO: 1, SEQ ID NO: 2, SEQ ID No. 7, SEQ ID NO:8, SEQ ID No. 13, and SEQ ID No. 14 under high, moderate or reduced stringency conditions as described above.

In still another embodiment, the invention relates to a complement of nucleic acid of NsG31. In particular, it relates to complements of the coding sequence of SEQ ID NO: 1, SEQ ID NO: 2, SEQ ID No. 7 SEQ ID No 8, SEQ ID No. 13, and SEQ ID No. 14.

In another embodiment, the invention relates to an RNA counterpart of the DNA nucleic acid of NsG31. In particular, it relates to RNA counterparts comprising the coding sequence of SEQ ID NO: 2, SEQ ID No 8, and SEQ ID No. 14.

Codon optimised nucleic acid molecules for enhanced expression in selected host cells, including but not limited to E. coli, yeast species, Chinese Hamster, Baby Hamster, insect, and fungus are also contemplated.

Variant nucleic acids can be made by state of the art mutagenesis methods. Methods for shuffling coding sequences from human with those of mouse, rat or chimpanzee are also contemplated. Specifically a shuffled variant may be between SEQ ID No 2 on one hand and 8 on the other hand. Also included are shuffled variants between SEQ ID No. 2 or 8 on one hand and a sequence coding for another Cys10 protein on the other hand.

In yet another embodiment, the invention relates to nucleic acid sequences (e.g., DNA, RNA) that hybridise to nucleic acids of NsG29. In particular, nucleic acids which hybridise to SEQ ID NO: 17 and/or SEQ ID NO: 22 under high, moderate or reduced stringency conditions as described above.

In still another embodiment, the invention relates to a complement of nucleic acid of NsG29. In particular, it relates to complements of SEQ ID NO: 17 and SEQ ID No 22.

In another embodiment, the invention relates to an RNA counterpart of the DNA nucleic acid of NsG29. In particular, it relates to RNA counterparts of SEQ ID NO: 17 and SEQ ID No 22.

Codon optimised nucleic acid molecules for enhanced expression in selected host cells, including but not limited to E. coli, yeast species, Chinese Hamster, Baby Hamster, insect, and fungus are also contemplated.

Variant nucleic acids can be made by state of the art mutagenesis methods. Methods for shuffling coding sequences from human with those of mouse, rat or chimpanzee are also contemplated. Specifically a shuffled variant may be between SEQ ID No 17 on one hand and 22 on the other hand. Also included are shuffled variants between SEQ ID No. 17 or 22 on one hand and a sequence coding for another Cys10 protein on the other hand.

### Va Use of NSG31 Polypeptides, Polynucleotides, and NsG31 Secreting Cells for Treatment of Disorders of the Nervous System

In one embodiment, native NsG31, variant NsG31, and fragments thereof and/or fusion proteins comprising NsG31 are provided for the treatment of disorders of the mammalian nervous system. NsG31 may be used to stimulate neural cell growth, proliferation, neural function, neural regeneration, neural differentiation, neural migration, and/or neural survival in disease situations where these cells are lost or damaged.

In one embodiment, polynucleotides and/or polypeptides of the invention may be used to treat conditions or diseases where neural growth, proliferation, differentiation, function, survival, and/or regeneration is desirable. The polypeptides of the present invention may be used directly via, e.g., injected, implanted or ingested pharmaceutical compositions to treat a pathological process responsive to the NsG31 polypeptides. This is supported by the bioinformatics analyses showing that NsG31 is a secreted growth factor and the fact that NsG31 is preferentially expressed in the nervous system, including the eye (FIGS. 5, 6, 7, and 8), the fact that mNsG31 is expressed differentially in the developing mouse CNS (FIG. 15) and the fact that the closely related growth factors, NsG30, NsG32a, and NsG32b, have shown a survival-enhancing effect in a cell line with neuronal potential (PC12 cells). In addition the closely related NsG28 and NsG32 have shown a survival enhancing effect on primary cerebellar granule cells.

NsG31 may act on a range of different cell types, which are present in the nervous system. In the context of the present invention, the nervous system is intended to encompass the central nervous system, the peripheral nervous system, the eye, and the cochleovestibular complex.

In one embodiment, NsG31 polypeptides may act on neurons, including but not limited to motor neurons, sensory neurons, relay cells, Purkinje cells, and interneurons.

In another embodiment, the therapeutic effect of NsG31 polypeptides may be through action on glial cells, such as oligodendrocytes and/or astrocytes. Through their action on glial cells, NsG31 polypeptides may be involved in myelination, and in the maintenance of neuron function and survival.

In another embodiment, NsG31 polypeptides may act on sensory cells, including but not limited to retinal ganglion cells, photoreceptor cells, supportive tissue such as retinal epithelial cells, and hair cells of the ear.

In a further embodiment, NsG31 polypeptides may act on stem cells, and downstream precursor cells including but not limited to neuronal precursors and glial precursors. NsG31 polypeptides may act on stem cells and/or neuronal or glial precursors to cause growth, proliferation, enhance survival, to cause differentiation, and/or migration. Stem cell therapy may be done through in vivo or ex vivo gene therapy, or in vitro treatment of isolated stem cells, or the protein may be administered to a location with stem cells. The effect of NsG31 on stem cells may be tested using the Neurosphere assay described herein (Example 12).

The disorder or disease or damage may be damages of the nervous system caused by trauma, surgery, ischaemia, infection, metabolic diseases, nutritional deficiency, malignancy or toxic agents, and genetic or idiopathic processes.

In one embodiment of the method of the invention, the disease or disorder or damage involves injury to the brain, brain stem, the spinal cord, and/or peripheral nerves, resulting in conditions such as stroke, traumatic brain injury (TBI), spinal cord injury (SCI), diffuse axonal injury (DAI), epilepsy, neuropathy, peripheral neuropathy, and associated pain and other symptoms that these syndromes may cause.

In another embodiment, the disease, disorder, or damage involves the degeneration of neurons and their processes in the brain, brain stem, the spinal cord, and/or peripheral nerves, such as neurodegenerative disorders including but not limited to Parkinson's Disease, Alzheimer's Disease, senile dementia, Huntington's Disease, amyotrophic lateral sclerosis (ALS), neuronal/axonal injury associated with Multiple Sclerosis (MS), and associated symptoms.

In another embodiment, the disease, disorder, or damage involves dysfunction, and/or loss of neurons in the brain, brain stem, the spinal cord, and/or peripheral nerves, such as dysfunction and/or loss caused by metabolic diseases, nutritional deficiency, toxic Injury, malignancy, and/or genetic or idiopathic conditions, including but not limited to diabetes, renal dysfunction, alcoholism, chemotherapy, chemical agents, drug abuse, vitamin deficiencies, infection, and associated symptoms.

In another embodiment, the disease, disorder, or damage involves the degeneration or sclerosis of glia such as oligodendrocytes, astrocytes, and Schwann cells in the brain, brain stem, the spinal cord, and peripheral nervous system, including but not limited to Multiple Sclerosis (MS), optic neuritis, cerebral sclerosis, post-infectious encephalomyelitis, and epilepsy, and associated symptoms.

In another embodiment, the disease, disorder, or damage involves the retina, photoreceptors, and associated nerves including but not limited to retinitis pigmentosa, macular degeneration, glaucoma, and associated symptoms.

In another embodiment, the disease, disorder, or damage involves the sensory epithelium and associated ganglia of the vestibuloacoustic complex, including but not limited to noise induced hearing loss, deafness, tinnitus, otitis, labyrintitis, hereditary and cochleovestibular atrophies, Meniere's Disease, and associated symptoms.

In a preferred embodiment, the polypeptides, nucleic acids, expression vectors, capsules and pharmaceutical compositions of the invention are used in the treatment of Parkinson's Disease. This function is based on the finding of high levels of expression in the central midbrain in substantia nigra and thalamus (see Example 4) and the finding of differential expression in the developing mouse VM and striatum (see Example 13) combined with the bioinformatics analyses. The function can be verified using the Bioassay for dopaminergic neurotrophic activities (example 10) and in vivo through the instrastriatal 6-OHDA lesion model (Example 11).

Huntington's disease (HD) is an autosomal dominant disorder that results in the progressive degeneration of various neuronal populations within the brain, particularly the GABA-ergic medium spiny neurons located in the caudate nucleus. Associated with this degeneration, the cortical glutaminergic input neurons also degenerate and the combined degeneration account for most of the characteristic symptoms of progressive dyskinetic motor movements as well as dementia.

In one embodiment, the polypeptides, nucleic acids, expression vectors, capsules and pharmaceutical compositions of the invention are used in the treatment of Huntington's disease. This is based on the finding of expression in the putamen and thalamus (Example 4), and the differential expression found in the developing mouse striatum and VM (Example 13) combined with the results of the bioinformatics analyses. Huntington's disease is an excitotoxic disease. An excitotoxic bioassay is the assay described in Example 6 of the present invention. Another exemplary bioassay for verification of this neuroprotective effect of NsG31 include e.g. the bioassay on protection of primary hippocampal slice cultures against the excitoxic effects of NMDA (WO 03/004527, example 5).

In another preferred embodiment, the polypeptides, nucleic acids, expression vectors, capsules and pharmaceutical compositions of the Invention are used in the treatment of peripheral neuropathies. This is based on the finding of high expression in the dorsal root ganglion combined with the results of the bioinformatics analyses. Verification of this function can be done with the dorsal root ganglion culture assay described in example 9. Among the peripheral neuropathies contemplated for treatment with the molecules of this invention are trauma-induced neuropathies, e.g., those caused by physical injury or disease state, physical damage to the peripheral nerves such as hermited discs, and the brain, physical damage to the spinal cord, stroke associated with brain damage, and neurological disorders related to neurodegeneration. We also contemplate treatment of chemotherapy-induced neuropathies (such as those caused by delivery of chemotherapeutic agents, e.g., taxol or cisplatin); toxin-induced neuropathies, drug-induced neuropathies, vitamin-deficiency-induced neuropathies; idiopathic neuropathies; and diabetic neuropathies.

In another preferred embodiment, the polypeptides, nucleic acids, expression vectors, capsules, and compositions of the Invention are used in the treatment of disorders, diseases, or damages associated with the Cerebellum, including but not limited to sensory ataxia, multiple sclerosis, neurodegenerative spinocerebellar disorders, hereditary ataxia, cerebellar atrophies (such as Olivopontocerebellar Atrophy (OPCA), Shy-Drager Syndrome (multiple systems atrophy)), and alcoholism. This function is supported by the high expression levels in the cerebellum combined with the bloinformatics analyses. Verification of this function may be done with the assays described in Examples 6 and 7 (Protection of cerebellar granule cells from glutamate toxicity and potassium deprivation).

In one embodiment, the polypeptides, nucleic acids, expression vectors, capsules and pharmaceutical compositions of the invention are used in the treatment of amyotrophic lateral sclerosis, spinal muscular atrophy, and spinal cord injury. This is based on the finding of expression in the spinal cord combined with the results of the bioinformatics analyses. Verification of this specific therapeutic function may be done with the motorneuron assay described in example 9.

In a preferred embodiment, the polypeptides, nucleic acids, vectors, capsules, and compositions of the invention are used in the treatment of thalamic pain. This function is based on the finding of high expression levels in the thalamus combined with the results of the bloinformatics analyses. Thalamic pain is a syndrome caused by stroke involving the thalamus.

In a preferred embodiment, the polypeptides, nucleic acids, vectors, capsules, and compositions of the invention are used in the treatment of essential tremor. This function is based on the finding of high expression levels in the thalamus combined with the results of the bioinformatics analyses. Essential tremor is an idiopathic syndrome, which can be treated by deep brain/thalamic stimulation in the thalamus and by thalamotomy.

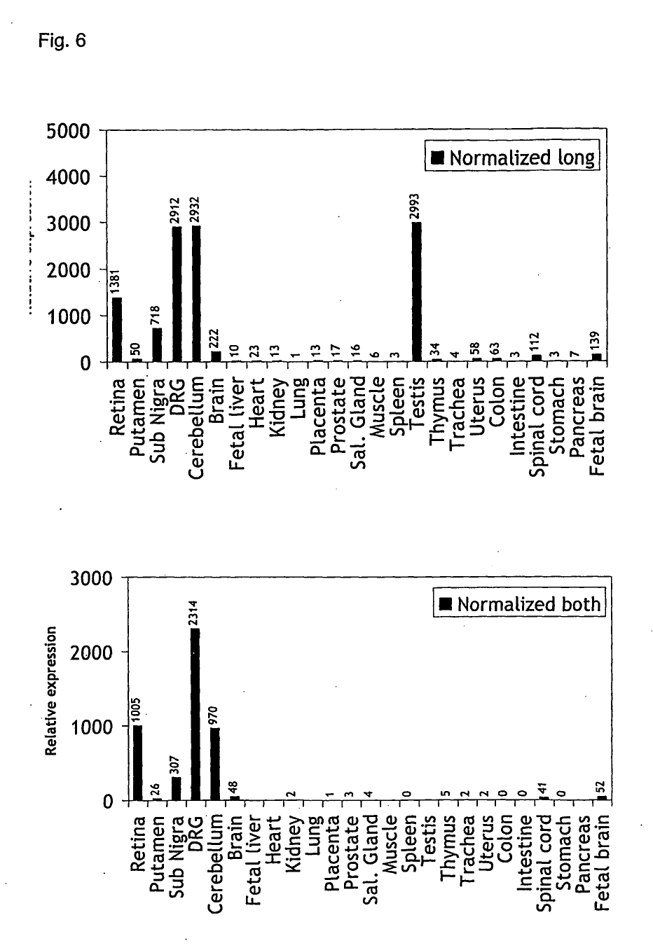

In a preferred embodiment, the polypeptides, nucleic acids, vectors, capsules, and compositions of the invention are used in the treatment of diseases, disorders, or damages involving the retina, including but not limited to retinitis pigmentosa, macular degeneration and glaucoma. This specific therapeutic use is supported by the bioinformatics and experimental analyses showing that NsG31 is a secreted growth factor highly expressed in the retina (FIGS. 5 and 6). Example 14 provides one assay for in vitro verification of this function.

Other growth factors have important therapeutic uses in both the central and peripheral nervous system and in various eye indications associated with loss of cells in retina and/or cornea. E.g NGF, is a candidate for both Alzheimer's disease, corneal ulcer (U.S. Pat. No. 6,063,757 and EP 0 973 872), and retinopathies. Neublastin (Artemin) is a candidate for both peripheral neuropathy (WO 02/078730) and corneal wound healing (EP 1 223 966). GDNF is a candidate for Parkinson's Disease, ALS, spinal cord injury, and for wound healing, in particular in cornea (EP 1 223 966).

Confirmation of such use can be obtained by using various state of the art in vitro assays (retinal explant assays, corneal cultures). Verification of function may also be performed in state of the art animal models for corneal wounds (corneal lesion in rabbits) and retina (retinitis pigmentosa mutant models available for mouse and rat).

In another embodiment the neurodegenerative disease is an excitotoxic disease selected from the group consisting of ischaemia, epilepsy, and trauma due to injury, cardiac arrest or stroke. The above-mentioned hippocampal slice culture assay and the assay of Example 6 of the present invention are non-limiting examples of an assay, which can be used to demonstrate a biological effect, indicative of therapeutic use for the treatment of excitotoxic diseases.

In another preferred embodiment the invention relates to a pathological condition related to testis. This embodiment is based on the present inventor's finding of high NsG31 expression in testis combined with the results of the bloinformatics analyses. Examples of diseases included within the scope of this embodiment include male sterility, impotence, erectile dysfunction, cancer, and germ cell tumours. NsG31 may also possess potential as a male contraceptive. Other growth factors with expression in both the central nervous system and testis have been shown to possess therapeutic potential in treating testes-related disorders, including use as a male contraceptive (WO 00/10594);

The term “subject” used herein is taken to mean any mammal to which NsG31 polypeptide or polynucleotide, therapeutic cells or biocompatible capsules may be administered. Subjects specifically intended for treatment with the method of the invention include humans, as well as nonhuman primates, sheep, horses, cattle, goats, pigs, dogs, cats, rabbits, guinea pigs, hamsters, gerbils, rats and mice, as well as the organs, tumors, and cells derived or originating from these hosts.

### Vb Use of NsG29 Polypeptides, Polynucleotides, and NsG29 Secreting Cells for Treatment of Disorders of the Nervous System

In one embodiment, native, variant NsG29, and fragments thereof and/or fusion proteins comprising NsG29 are provided for the treatment of disorders of the mammalian nervous system. NsG29 may be used to stimulate neural cell growth including proliferation, neural function, neural regeneration, neural differentiation, neural migration, and/or neural survival in disease situations where these cells are lost or damaged.

In one embodiment, polynucleotides and/or polypeptides of the invention may be used to treat conditions or diseases where neural growth including proliferation, differentiation, function, survival, and/or regeneration is desirable. The polypeptides of the present invention may be used directly via, e.g., injected, implanted or ingested pharmaceutical compositions to treat a pathological process responsive to the NsG29 polypeptides. This is supported by the bioinformatics analyses showing that NsG29 is a secreted growth factor and the fact that NsG29 is preferentially expressed in the nervous system, including the eye (FIGS. 19 and 20), the fact that the expression of mouse NsG29 is developmentally regulated in the developing mouse CNS, and the fact that the closely related growth factors, NsG30, NsG32a, and NsG32b, have shown a survival-enhancing (antiapoptotic) effect in a cell line with neuronal potential (PC12 cells). A survival enhancing (antiapoptotic) effect has also been demonstrated by the closely related growth factors NsG28 and NsG32a on primary mouse cerebellar granule cells.

NsG29 may act on a range of different cell types, which are present in the nervous system. In the context of the present invention, the nervous system is intended to encompass the central nervous system, the peripheral nervous system, the eye, and the cochleovestibular complex.

In one embodiment, NsG29 polypeptides may act on neurons, including but not limited to motor neurons, sensory neurons, relay cells, Purkinje cells, and interneurons.

In another embodiment, the therapeutic effect of NsG29 polypeptides may be through action on glial cells, such as oligodendrocytes and/or astrocytes. Through their action on glial cells, NsG29 polypeptides may be involved in myelination, and in the maintenance of neuron function and survival. The involvement in myelination is supported by the temporal and spatial expression in the developing mouse CNS.

In another embodiment, NsG29 polypeptides may act on sensory cells, including but not limited to retinal ganglion cells, photoreceptor cells, supportive tissue such as retinal epithelial cells, and hair cells of the ear.

In a further embodiment, NsG29 polypeptides may act on stem cells, and downstream precursor cells including but not limited to neuronal precursors and glial precursors. NsG29 polypeptides may act on stem cells and/or neuronal or glial precursors to cause growth, proliferation, enhance survival, to cause differentiation, and/or migration. Stem cell therapy may be done through in vivo or ex vivo gene therapy, or in vitro treatment of isolated stem cells, or the protein may be administered to a location with stem cells. The effect on stem cells is supported by the expression in the adult human amygdale and hippocampus as these regions show extensive plasticity and stem cell proliferation during adulthood. The effect of NsG29 on stem cells may be tested using the Neurosphere assay described herein (Example 12).

The disorder or disease or damage may be damages of the nervous system caused by trauma, surgery, ischaemia, infection, metabolic diseases, nutritional deficiency, malignancy or toxic agents, and genetic or idiopathic processes.

In one embodiment of the method of the invention, the disease or disorder or damage involves Injury to the brain, brain stem, the spinal cord, and/or peripheral nerves, resulting in conditions such as stroke, traumatic brain injury (TBI), spinal cord Injury (SCI), diffuse axonal injury (DAI), epilepsy, neuropathy, peripheral neuropathy, and associated pain and other symptoms that these syndromes may cause.

In another embodiment, the disease, disorder, or damage Involves the degeneration of neurons and their processes in the brain, brain stem, the spinal cord, and/or peripheral nerves, such as neurodegenerative disorders including but not limited to Parkinson's Disease, Alzheimer's Disease, senile dementia, Huntington's Disease, amyotrophic lateral sclerosis (ALS), neuronal/axonal injury associated with Multiple Sclerosis (MS), and associated symptoms. Preferably, the disorder is multiple sclerosis.

In another embodiment, the disease, disorder, or damage involves dysfunction, and/or loss of neurons in the brain, brain stem, the spinal cord, and/or peripheral nerves, such as dysfunction and/or loss caused by metabolic diseases, nutritional deficiency, toxic injury, malignancy, and/or genetic or idiopathic conditions, including but not limited to diabetes, renal dysfunction, alcoholism, chemotherapy, chemical agents, drug abuse, vitamin deficiencies, infection, and associated symptoms.

In another embodiment, the disease, disorder, or damage involves the degeneration or sclerosis of glia such as oligodendrocytes, astrocytes, and Schwann cells in the brain, brain stem, the spinal cord, and peripheral nervous system, including but not limited to Multiple Sclerosis (MS), optic neuritis, cerebral sclerosis, post-infectious encephalomyelitis, and epilepsy, and associated symptoms.

In another embodiment, the disease, disorder, or damage involves the retina, photoreceptors, and associated nerves including but not limited to retinitis pigmentosa, macular degeneration, glaucoma, and associated symptoms.

In another embodiment, the disease, disorder, or damage involves the sensory epithelium and associated ganglia of the vestibuloacoustic complex, including but not limited to noise induced hearing loss, deafness, tinnitus, otitis, labyrintitis, hereditary and cochleovestibular atrophies, Meniere's Disease, and associated symptoms.

In a preferred embodiment, the polypeptides, nucleic acids, expression vectors, capsules and pharmaceutical compositions of the invention are used in the treatment of Parkinson's Disease. This function is based on the finding of high levels of expression in the central midbrain in substantia nigra and the putamen (see Example 4b), the finding of expression in the developing human mesencephalon (See Example 4b); and the spatial and temporal expression in the developing mouse CNS (see example 13b). The function can be verified using the Bioassay for dopaminergic neurotrophic activities (example 10) and in vivo through the instrastriatal 6-OHDA lesion model (Example 11).

The most consistent abnormality for Alzheimer's disease, as well as for vascular dementia and cognitive impairment due to organic brain disease related to alcoholism, is the degeneration of the cholinergic system arising from the basal forebrain (BF) to both the codex and hippocampus (Bigl et al. in Brain Cholinergic Systems, M. Steriade and D. Biesold, eds., Oxford University Press, Oxford, pp. 364-386 (1990)). In chronic alcoholism the resultant organic brain disease, like Alzheimer's disease and normal aging, is also characterized by diffuse reductions in cortical cerebral blood flow in those brain regions where cholinergic neurons arise (basal forebrain) and to which they project (cerebral cortex) (Lofti et al., Cerebrovasc. and Brain Metab. Rev 1:2 (1989)). Therefore, in a preferred embodiment, the polypeptides, nucleic acids, expression vectors, capsules and pharmaceutical compositions of the invention are used in the treatment of Alzheimer's Disease. This function is based on the finding of high levels of expression in adult human hippocampus and amygdala combined with the results of the bioinformatics analyses. The therapeutic potential can be tested in an in vitro assay with basal cholinergic forebrain neurons, which are subjected to conditioned medium as described in the examples. An increase in ChAT (choline acetyltransferase) activity in basal cholinergic forebrain neurons is an indication of therapeutic effect in the treatment of Alzheimers disease. Another relevant assay is an in vitro assay with cortical neurons.

Huntington's disease (HD) is an autosomal dominant disorder that results in the progressive degeneration of various neuronal populations within the brain, particularly the GABA-ergic medium spiny neurons located in the caudate nucleus. Associated with this degeneration, the cortical glutaminergic input neurons also degenerate and the combined degeneration account for most of the characteristic symptoms of progressive dyskinetic motor movements as well as dementia.

In a preferred embodiment, the polypeptides, nucleic acids, expression vectors, capsules and pharmaceutical compositions of the invention are used in the treatment of Huntington's disease. This is based on the finding of high expression in the putamen combined with the results of the bloinformatics analyses. This function is further supported by the finding of expression in the developing mouse striatum (See example 13b). Huntington's disease is an excitotoxic disease. An excitotoxic bioassay is the assay described in Example 6 of the present invention. Another exemplary bioassay for verification of this neuroprotective effect of NsG29 include e.g. the bioassay on protection of primary hippocampal slice cultures against the excitoxic effects of NMDA (WO 03/004527, example 5). Another relevant assay is an in vitro assay with cortical neurons.

In another preferred embodiment, the polypeptides, nucleic acids, expression vectors, capsules and pharmaceutical compositions of the invention are used in the treatment of peripheral neuropathies. This is based on the finding of high expression in the dorsal root ganglion combined with the results of the bioinformatics analyses. Verification of this function can be done with the dorsal root ganglion culture assay described in example 9. Among the peripheral neuropathies contemplated for treatment with the molecules of this invention are trauma-induced neuropathies, e.g., those caused by physical Injury or disease state, physical damage to the peripheral nerves such as hermited discs, and the brain, physical damage to the spinal cord, stroke associated with brain damage, and neurological disorders related to neurodegeneration. We also contemplate treatment of chemotherapy-induced neuropathies (such as those caused by delivery of chemotherapeutic agents, e.g., taxol or cisplatin); toxin-induced neuropathies, drug-induced neuropathies, vitamin-deficiency-induced neuropathies; idiopathic neuropathies; and diabetic neuropathies.

In another embodiment, the polypeptides, nucleic acids, expression vectors, capsules, and compositions of the Invention are used in the treatment of disorders, diseases, or damages associated with the Cerebellum, including but not limited to sensory ataxia, multiple sclerosis, neurodegenerative spinocerebellar disorders, hereditary ataxia, cerebellar atrophies (such as Olivopontocerebellar Atrophy (OPCA), Shy-Drager Syndrome (multiple systems atrophy)), and alcoholism. This function is supported by expression in the adult human cerebellum and in the developing but not the adult mouse cerebellum, combined with the bioinformatics analyses. Verification of this function may be done with the assays described in Examples 6 and 7 (Protection of cerebellar granule cells from glutamate toxicity and potassium deprivation).

In another embodiment, the polypeptides, nucleic acids, expression vectors, capsules and pharmaceutical compositions of the invention are used in the treatment of amyotrophic lateral sclerosis, spinal muscular atrophy, and spinal cord injury. This is based on the finding of expression in the adult human spinal cord and the developing mouse spinal cord, combined with the results of the bioinformatics analyses. Verification of this specific therapeutic function may be done with the motorneuron assay described in example 9. Another relevant assay is an in vitro assay with cortical neurons.

In one embodiment, the polypeptides, nucleic acids, vectors, capsules, and compositions of the invention are used in the treatment of diseases, disorders, or damages involving the retina, including but not limited to retinitis pigmentosa, macular degeneration and glaucoma. This specific therapeutic use is supported by the bioinformatics and experimental analyses showing that NsG29 is a secreted growth factor expressed in the retina (FIG. 19). Example 14 provides one assay for in vitro verification of this function.

Other growth factors have important therapeutic uses in both the central and peripheral nervous system and in various eye indications associated with loss of cells in retina and/or cornea. E.g NGF, is a candidate for both Alzheimer's disease, corneal ulcer (U.S. Pat. No. 6,063,757 and EP 0 973 872), and retinopathies. Neublastin (Artemin) is a candidate for both peripheral neuropathy (WO 02/078730) and corneal wound healing (EP 1 223 966). GDNF is a candidate for Parkinson's Disease, ALS, spinal cord injury, and for wound healing, in particular in cornea (EP 1 223 966). Confirmation of such use can be obtained by using various state of the art in vitro assays (retinal explant assays, corneal cultures). Verification of function may also be performed in state of the art animal models for corneal wounds (corneal lesion in rabbits) and retina (retinitis pigmentosa mutant models available for mouse and rat).

In another preferred embodiment, the polypeptides, nucleic acids, expression vectors, capsules, and compositions of the Invention are used in the treatment of multiple sclerosis. This function is based on the finding of expression in corpus callosum as well as the spinal cord, combined with the results of the bioinformatics analyses. The corpus callosum consists primarily of glial cells. Expression in the corpus callosum thus indicates a strong association with glia. The potential function in MS therapy is further supported by the interpretation of the expression in the developing mouse CNS (example 13b).

In one embodiment, the polypeptides, nucleic acids, vectors, capsules, and compositions of the invention are used in the treatment of thalamic pain. This function is based on the finding of intermediate expression levels in the thalamus combined with the results of the bioinformatics analyses. Thalamic pain is a syndrome caused by stroke involving the thalamus.

In one embodiment, the polypeptides, nucleic acids, vectors, capsules, and compositions of the invention are used in the treatment of essential tremor. This function is based on the finding of Intermediate expression levels in the thalamus combined with the results of the bioinformatics analyses. Essential tremor is an Idiopathic syndrome, which can be treated by deep brain/thalamic stimulation in the thalamus and by thalamotomy.

In another embodiment the neurodegenerative disease is an excitotoxic disease selected from the group consisting of ischaemia, epilepsy, and trauma due to injury, cardiac arrest or stroke. The above-mentioned hippocampal slice culture assay and the assay of Example 6 of the present invention are non-limiting examples of an assay, which can be used to demonstrate a biological effect, indicative of therapeutic use for the treatment of excitotoxic diseases.

The term “subject” used herein is taken to mean any mammal to which NsG29 polypeptide or polynucleotide, therapeutic cells or biocompatible capsules may be administered. Subjects specifically intended for treatment with the method of the Invention include humans, as well as nonhuman primates, sheep, horses, cattle, goats, pigs, dogs, cats, rabbits, guinea pigs, hamsters, gerbils, rats and mice, as well as the organs, tumors, and cells derived or originating from these hosts.

### VI. Polypeptide Administration and Formulations

A target tissue for NsG29 and/or NsG31 therapy is a region selected for its retained responsiveness to NsG29 and/or NsG31. In humans, neurons, which retain responsiveness to growth factors into adulthood include the cholinergic basal forebrain neurons, the entorhinal cortical neurons, the thalamic neurons, the locus coeruleus neurons, the spinal sensory neurons, the spinal motor neurons, neurons of substantia nigra, sympathetic neurons, dorsal root ganglia, retina neurons, otic neurons, cerebellar neurons, and ciliary ganglia. Stem cells, such as stem cells of the subventricular zone, and neural and glial progenitor cells also retain responsiveness to growth factors into adulthood. Also myelinating oligodendrocytes retain responsiveness to growth factors into adulthood.

NsG29 and/or NsG31 polypeptides may be administered in any manner, which is medically acceptable. This may include Injections, by parenteral routes such as intravenous, intravascular, intraarterial, subcutaneous, intramuscular, intratumor, intraperitoneal, intraventricular, intraepidural, intertracheal, intrathecal, intracerebroventricular, intercerebral, interpulmonary, or others as well as nasal, ophthalmic, rectal, or topical. Sustained release administration is also specifically included in the invention, by such means as depot injections or erodible implants. Peroral administration is also conceivable provided the protein is protected against degradation in the stomach.

Administration of an NsG29 and/or NsG31 according to this invention may be achieved using any suitable delivery means, including:

pump (see, e.g., Annals of Pharmacotherapy, 27:912 (1993); Cancer, 41:1270 (1993); Cancer Research, 44:1698 (1984), incorporated herein by reference),

microencapsulation (see, e.g., U.S. Pat. Nos. 4,352,883; 4,353,888; and 5,084,350, herein incorporated by reference),

continuous release polymer implants (see, e.g., Sabel, U.S. Pat. No. 4,883,666, incorporated herein by reference),

encapsulated cells (see, Section X),

naked or unencapsulated cell grafts to the CNS (see, e.g., U.S. Pat. Nos. 5,082,670 and 5,618,531, each incorporated herein by reference);

injection, either subcutaneously, intravenously, intra-arterially, intramuscularly, or to other suitable site;

inhalation; and

oral administration, in capsule, liquid, tablet, pill, or prolonged release formulation.

Administration may be by periodic injections of a bolus of the preparation, or may be made more continuous by intravenous or intraperitoneal administration from a reservoir which is external (e.g., an IV bag) or internal (e.g., a bioerodable implant, a bioartificial organ, a biocompatible capsule of NsG29 and/or NsG31 production cells, or a colony of implanted NsG29 and/or NsG31 production cells). See, e.g., U.S. Pat. Nos. 4,407,957, 5,798,113, and 5,800,828, each incorporated herein by reference. Intrapulmonary delivery methods and apparatus are described, for example, in U.S. Pat. Nos. 5,654,007, 5,780,014, and 5,814,607, each incorporated herein by reference.

Apart from systemic delivery, delivery directly to the CNS or the eye behind the blood-brain or blood-retina barriers is also contemplated.

Localised delivery may be by such means as delivery via a catheter to one or more arteries, such as the ophthalmic artery to the eye, and the cerebral artery to the CNS. Methods for local pump-based delivery of protein formulations to the CNS are described in U.S. Pat. No. 6,042,579 (Medtronic). Another type of localised delivery comprises delivery using encapsulated cells (see Section XI). A further type of localised delivery comprises local delivery of gene therapy vectors, which are normally injected.

For the treatment of eye disorders, delivery may be systemic, or local such as delivery via the ophthalmic artery. In another embodiment, delivery is via Encapsulated Cell Therapy, where the encapsulated cells are implanted intravitreally. Delivery of protein formulations or gene therapy vector may be done using subretinal injections, intravitreal injection, or transcleral injection.

For the treatment of Parkinson's Disease, various delivery routes can be taken. Protein formulations can be administered with pumps intracerbroventricularly or intraparenchymally, preferably to the striatum and/or substantia nigra, more preferably to the intraputamen. However, a more preferred delivery method comprises encapsulated cell therapy, where the capsules are implanted intracerebroventricularly, or intraparenchymally, preferably into the striatum, and/or substantia nigra, and more preferably into the putamen. In one embodiment relating to treatment of Parkinson's Disease, gene therapy vector is administered to the striatum of the brain. Injection into the striatum can label target sites located in various distant regions of the brain, for example, the globus pallidus, amygdala, subthalamic nucleus or the substantia nigra. Transduction of cells in the pallidus commonly causes retrograde labelling of cells in the thalamus. In a preferred embodiment the (or one of the) target site(s) is the substantia nigra.

In an embodiment to treat HD, NsG29 and/or NsG31 is applied to the striatum, preferably the caudate nucleus in order to protect the neurons from degeneration, resulting in both protection of the caudate neurons and the cortical input neurons. In a preferred embodiment, the application should occur before the onset of major degenerative changes. The treatment would involve the genetic diagnosis of the disease through family history and DNA analysis of the blood followed by the local application of NsG29 and/or NsG31. This would be accomplished by delivering the NsG29 and/or NsG31 to the striatum via pumping of the protein with the use of medically applicable infusion pumps and catheters, e.g. Medtronic Synchrotron pump. In a second strategy, direct gene therapy using viral or non-viral vectors could be utilized to modify the host cells in the striatum or other affected neurons to secrete NsG29 and/or NsG31. In a third strategy, naked or encapsulated cells genetically modified to make and secrete NsG29 and/or NsG31 can be applied locally to deliver NsG29 and/or NsG31 behind the blood-brain-barrier and within the diseased region, preferably the striatum, even more preferred, the caudate nucleus.

In ALS, both upper and lower motor neurons degenerate, causing progressive paralyses, eventually leading to death, most commonly through respiratory complications. To treat ALS, NsG29 and/or NsG31 would be delivered to the CNS including the spinal cord through the infusion of NsG29 and/or NsG31 into the lumbar intrathecal space thereby mixing with the cerebrospinal fluid (CSF), which bathes the spinal cord and brain. The delivery could be accomplished through the implantation of pump and catheters, e.g. Medtronic Synchrotron pump or through the use of encapsulated cell devices implanted into the lumbar inthrathecal space. Direct gene therapy could also be used by injecting DNA carrying vectors into the CSF, thereby transferring the gene to cells lining the CSF space. In addition, gene transfer vectors can be injected into the cervical or lumbar spinal cord or intracerebral, thereby secreting NsG29 and/or NsG31 in the anatomical regions containing the majority of the motor neurons involved in motor paralyses and respiratory function. These injections would occur under surgical navigation and could be performed relatively safely.

In subjects with neurodegenerative diseases such as AD, neurons in the Ch4 region (nucleus basalis of Meynert) which have nerve growth factor (NGF) receptors undergo marked atrophy as compared to normal controls (see, e.g., Kobayashi, et al., Mol. Chem. Neuropathol., 15: 193-206 (1991)). In normal subjects, neurotrophins prevent sympathetic and sensory neuronal death during development and prevents cholinergic neuronal degeneration in adult rats and primates (Tuszynski, et al., Gene Therapy, 3: 305314 (1996)). The resulting loss of functioning neurons in this region of the basal forebrain is believed to be causatively linked to the cognitive decline experienced by subjects suffering from neurodegenerative conditions such as AD (Tuszynski, et al., supra and, Lehericy, et al., J. Comp. Neurol., 330: 15-31 (1993)). In general it is contemplated, that AD can be treated with NsG29 and/or NsG31 protein formulations delivered intracerebroventricularly, or intraparenchymally. Within the intraparenchymal area, delivery is preferably to the basal forebrain, and to the hippocampus. Gene therapy vector, encapsulated or naked cells secreting NsG29 and/or NsG31 can also be administered to the basal forebrain or the hippocampus.

For the treatment of spinal cord injury, protein, gene therapy vector or encapsulated or naked cells secreting NsG29 and/or NsG31 can be delivered intrathecally at the position of the Injury as described above for the treatment of ALS.

For the treatment of peripheral neuropathy, delivery is either systemic (using protein formulations), intrathecally using protein formulations, gene therapy vectors, or encapsulated or naked cells secreting NsG29 and/or NsG31, or intramuscularly depending on retrograde transport to the spinal cord.

For the treatment of multiple sclerosis, delivery is either systemic (using protein formulations), or intraventricular, intrathecal or intralessional using protein formulations, gene therapy vectors, or encapsulated or naked cells secreting NsG29 and/or NsG31.

For the treatment of epilepsy NsG29 and/or NsG31 protein could be delivered intraparenchymally in the epilepsy focus. This may be done with encapsulated or naked cells, with protein formulation administered with catheter or pump or with gene therapy vector delivered to this site.

For the treatment of stroke or trauma, delivery is intrathecal, intracerbroventricular, or preferably intralessionar.

The term “pharmaceutically acceptable carrier” means one or more organic or inorganic ingredients, natural or synthetic, with which NsG29 and/or NsG31 polypeptide is combined to facilitate its application. A suitable carrier includes sterile saline although other aqueous and non-aqueous isotonic sterile solutions and sterile suspensions known to be pharmaceutically acceptable are known to those of ordinary skill in the art. An “effective amount” refers to that amount which is capable of ameliorating or delaying progression of the diseased, degenerative or damaged condition. An effective amount can be determined on an individual basis and will be based, in part, on consideration of the symptoms to be treated and results sought. An effective amount can be determined by one of ordinary skill in the art employing such factors and using no more than routine experimentation.

A liposome delivery system may be any variety of unilamellar vesicles, multilamellar vesicles, or stable plurilamellar vesicles, and may be prepared and administered according to methods well known to those of skill in the art, for example in accordance with the teachings of U.S. Pat. Nos. 5,169,637, 4,762,915, 5,000,958 or 5,185,154. In addition, it may be desirable to express the novel polypeptides of this invention, as well as other selected polypeptides, as lipoproteins, in order to enhance their binding to liposomes. A recombinant NsG29 and/or NsG31 protein is purified, for example, from CHO cells by immunoaffinity chromatography or any other convenient method, then mixed with liposomes and incorporated into them at high efficiency. The liposome-encapsulated protein may be tested in vitro for any effect on stimulating cell growth.

Any of the NsG29 and/or NsG31 polypeptides of this invention may be used in the form of a pharmaceutically acceptable salt. Suitable acids and bases which are capable of forming salts with an NsG29 and/or NsG31 polypeptide are well known to those of skill in the art, and include inorganic and organic acids and bases.

In addition to the active ingredients, the pharmaceutical compositions may comprise suitable ingredients. Further details on techniques for formulation and administration may be found in the latest edition of Remington's Pharmaceutical Sciences (Maack Publishing Co., Easton, Pa.).

Various dosing regimes for systemic administration are contemplated. In one embodiment, methods of administering to a subject a formulation comprising an NsG29 and/or NsG31 polypeptide include administering NsG29 and/or NsG31 at a dosage of between 1 μg/kg to 30,000 μg/kg body weight of the subject, per dose. In another embodiment, the dosage is between 10 μg/kg to 30,000 μg/kg body weight of the subject, per dose. In a further embodiment, the dosage is between 10 μg/kg to 10,000 μg/kg body weight of the subject, per dose. In a different embodiment, the dosage is between 25 μg/kg to 10,000 μg/kg body weight of the subject, per dose. In yet another embodiment, the dosage is between 25 μg/kg to 3,000 μg/kg body weight of the subject, per dose. In a most preferable embodiment, the dosage is between 50 μg/kg to 3,000 μg/kg body weight of the subject, per dose.

Guidance as to particular dosages and methods of delivery is provided in the literature; see, for example, U.S. Pat. No. 4,657,760; 5,206,344; or 5,225,212. It is anticipated that different formulations will be effective for different treatment compounds and different disorders, that administration targeting one organ or tissue, for example, may necessitate delivery in a manner different from that to another organ or tissue.

Where sustained-release administration of an NsG29 and/or NsG31 polypeptide is desired in a formulation with release characteristics suitable for the treatment of any disease or disorder requiring administration of an NsG29 and/or NsG31 polypeptide, microencapsulation of an NsG29 and/or NsG31 polypeptide is contemplated. Microencapsulation of recombinant proteins for sustained release has been successfully performed with human growth hormone (rhGH), interferon-(rhIFN-), interleukin-2, and MN rgp120. Johnson et al., Nat. Med., 2:795-799 (1996); Yasuda, Biomed. Ther., 27:1221-1223 (1993); Hora et al., Bio/Technology, 8:755-758 (1990); Cleland, “Design and Production of Single Immunization Vaccines Using Polylactide Polyglycolide Microsphere Systems,” in Vaccine Design: The Subunit and Adjuvant Approach, Powell and Newman, eds, (Plenum Press: New York, 1995), pp. 439-462; WO 97/03692, WO 96/40072, WO 96/07399; and U.S. Pat. No. 5,654,010.

The sustained-release formulations of these proteins were developed using poly-lactic-coglycolic acid (PLGA) polymer due to its biocompatibility and wide range of biodegradable properties. The degradation products of PLGA, lactic and glycolic acids, can be cleared quickly within the human body. Moreover, the degradability of this polymer can be adjusted from months to years depending on its molecular weight and composition. Lewis, “Controlled release of bioactive agents from lactide/glycolide polymer,” in: M. Chasin and R. Langer (Eds.), Biodegradable Polymers as Drug Delivery Systems (Marcel Dekker: New York, 1990), pp. 141.

The dose administered should be carefully adjusted to the age, weight and condition of the individual being treated, as well as the route of administration, dosage form and regimen, and the result desired, and the exact dosage should be determined by the practitioner.

### VII. Pharmaceutical Preparations for Gene Therapy

To form an NsG29 and/or NsG31 composition for gene therapy use in the invention, NsG29 and/or NsG31 encoding expression viral vectors may be placed into a pharmaceutically acceptable suspension, solution or emulsion. Suitable mediums include saline and liposomal preparations.

More specifically, pharmaceutically acceptable carriers may include sterile aqueous of non-aqueous solutions, suspensions, and emulsions. Examples of nonaqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringers dextrose, dextrose and sodium chloride, lactated Ringer's or fixed oils.

Intravenous vehicles include fluid and nutrient replenishers, electrolyte replenishers (such as those based on Ringer's dextrose), and the like.

Preservatives and other additives may also be present such as, for example, antimicrobials, antioxidants, chelating agents, and Inert gases and the like. Further, a composition of NsG29 and/or NsG31 transgenes may be lyophilized using means well known in the art, for subsequent reconstitution and use according to the invention.

A colloidal dispersion system may also be used for targeted gene delivery. Colloidal dispersion systems include macromolecule complexes, nanocapsules, microspheres, beads, and lipid-based systems including oil-in-water emulsions, micelles, mixed micelles, and liposomes. Liposomes are artificial membrane vesicles which are useful as delivery vehicles in vitro and in vivo. It has been shown that large unilamellar vesicles (LUV), which range in size from 0.2-4.0 μm can encapsulate a substantial percentage of an aqueous buffer containing large macro molecules. RNA, DNA and intact virions can be encapsulated within the aqueous interior and be delivered to cells in a biologically active form (Fraley, et al., Trends Biochem. Sci., 6: 77, 1981). In addition to mammalian cells, liposomes have been used for delivery of operatively encoding transgenes in plant, yeast and bacterial cells. In order for a liposome to be an efficient gene transfer vehicle, the following characteristics should be present: (1) encapsulation of the genes encoding the NsG29 and/or NsG31 at high efficiency while not compromising their biological activity; (2) preferential and substantial binding to a target cell in comparison to non-target cells; (3) delivery of the aqueous contents of the vesicle to the target cell cytoplasm at high efficiency; and (4) accurate and effective expression of genetic information (Mannino, et al., Biotechniques, 6: 682, 1988).

The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations.

Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Particularly useful are diacylphosphatidylglycerols, where the lipid moiety contains from 14-18 carbon atoms, particularly from 16-18 carbon atoms, and is saturated. Illustrative phospholipids include egg phosphatidylcholine, dipalmitoylphosphatidylcholine and distearoylphosphatidylcholine.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs which contain sinusoidal capillaries.

Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted gene delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand.

A further example of a delivery system includes transplantation into the therapeutic area of a composition of packaging cells capable of producing vector particles as described in the present invention. Methods for encapsulation and transplantation of such cells are known in the art, in particular from WO 97/44065 (Cytotherapeutics). By selecting a packaging cell line capable of producing lentiviral particles, transduction of non-dividing cells in the therapeutic area is obtained. By using retroviral particles capable of transducing only dividing cells, transduction is restricted to de-novo differentiated cells in the therapeutic area.

### VIII. Dosing Requirements and Delivery Protocol for Gene Therapy

An important parameter is the dosage of NsG29 and/or NsG31 gene therapy vector to be delivered into the target tissue. For viral vectors, the concentration may be defined by the number of transducing units/ml. Optimally, for delivery using a viral expression vector, each unit dosage will comprise 2.5 to 25 μL of a composition, wherein the composition includes a viral expression vector in a pharmaceutically acceptable fluid and provides from 10⁸ up to 10¹⁰ NsG29 and/or NsG31 transducing units per ml.

Importantly, specific in vivo gene delivery sites are selected so as to cluster in an area of loss, damage, or dysfunction of neural cells, glial cells, retinal cells, sensory cells, or stem cells. Such areas may be identified clinically using a number of known techniques, including magnetic resonance imaging (MRI) and biopsy. In humans, non-invasive, in vivo imaging methods such as MRI will be preferred. Once areas of neuronal loss are identified, delivery sites are selected for stereotaxic distribution so each unit dosage of NsG29 and/or NsG31 is delivered into the brain at, or within 500 μm from, a targeted cell, and no more than about 10 mm from another delivery site.

Within a given target site, the vector system may transduce a target cell. The target cell may be a cell found in nervous tissue, such as a neuron, astrocyte, oligodendrocyte, microglia, stem cells, neural precursor cells, or ependymal cell.

The vector system is preferably administered by direct injection. Methods for injection into the brain are well known in the art (Bilang-Bleuel et al (1997) Proc. Acad. Natl. Sci. USA 94:8818-8823; Choi-Lundberg et al (1998) Exp. Neurol. 154:261-275; Choi-Lundberg et al (1997) Science 275:838-841; and Mandel et al (1997)) Proc. Acad. Natl. Sci. USA 94:14083-14088). Stereotaxic injections may be given.

As mentioned above, for transduction in tissues such as the brain, it is necessary to use very small volumes, so the viral preparation is concentrated by ultracentrifugation. The resulting preparation should have at least 10⁸ t.u./ml, preferably from 10⁸ to 10¹⁰ t.u./ml, more preferably at least 10⁹ t.u./ml. (The titer is expressed in transducing units per ml (t.u./ml) as described in example 6). It has been found that improved dispersion of transgene expression can be obtained by increasing the number of injection sites and decreasing the rate of injection (Horellou and Mallet (1997) as above). Usually between 1 and 10 Injection sites are used, more commonly between 2 and 6. For a dose comprising 1-5×10⁹ t.u./ml, the rate of injection is commonly between 0.1 and 10 μl/min, usually about 1 μl/min.

The virus composition is delivered to each delivery cell site in the target tissue by microinjection, infusion, scrape loading, electroporation or other means suitable to directly deliver the composition directly into the delivery site tissue through a surgical incision. The delivery is accomplished slowly, such as over a period of about 5-10 minutes (depending on the total volume of virus composition to be delivered).

### IX. Viral Vectors

Broadly, gene therapy seeks to transfer new genetic material to the cells of a patient with resulting therapeutic benefit to the patient. Such benefits include treatment or prophylaxis of a broad range of diseases, disorders and other conditions.

Ex vivo gene therapy approaches involve modification of isolated cells (including but not limited to stem cells, neural and glial precursor cells, and foetal stem cells), which are then infused, grafted or otherwise transplanted into the patient. See, e.g., U.S. Pat. Nos. 4,868,116, 5,399,346 and 5,460,959. In vivo gene therapy seeks to directly target host patient tissue in vivo.

Viruses useful as gene transfer vectors include papovavirus, adenovirus, vaccinia virus, adeno-associated virus, herpesvirus, and retroviruses. Suitable retroviruses include the group consisting of HIV, SIV, FIV, EIAV, MoMLV.

Preferred viruses for treatment of disorders of the nervous system are lentiviruses and adeno-associated viruses. Both types of viruses can integrate into the genome without cell divisions, and both types have been tested in pre-clinical animal studies for indiations of the nervous system, in particular the central nervous system.

Methods for preparation of MV are described in the art, e.g. U.S. Pat. No. 5,677,158. U.S. Pat. No. 6,309,634 and U.S. Pat. No. 6,683,058 describe examples of delivery of MV to the central nervous system.

Preferably, a lentivirus vector is a replication-defective lentivirus particle. Such a lentivirus particle can be produced from a lentiviral vector comprising a 5′ lentiviral LTR, a tRNA binding site, a packaging signal, a promoter operably linked to a polynucleotide signal encoding said fusion protein, an origin of second strand DNA synthesis and a 3′ lentiviral LTR. Methods for preparation and in vivo administration of lentivirus to neural cells are described in US 20020037281 (Methods for transducing neural cells using lentiviral vectors).

Retroviral vectors are the vectors most commonly used in human clinical trials, since they carry 7-8 kb and since they have the ability to infect cells and have their genetic material stably integrated into the host cell with high efficiency. See, e.g., WO 95/30761; WO 95/24929. Oncovirinae require at least one round of target cell proliferation for transfer and integration of exogenous nucleic acid sequences into the patient. Retroviral vectors integrate randomly into the patient's genome. Retroviruses can be used to target stem cells of the nervous system as very few cell divisions take place in other cells of the nervous system (in particular the CNS).

Three classes of retroviral particles have been described; ecotropic, which can infect murine cells efficiently, and amphotropic, which can infect cells of many species. The third class includes xenotrophic retrovirus which can infect cells of another species than the species which produced the virus. Their ability to integrate only into the genome of dividing cells has made retroviruses attractive for marking cell lineages in developmental studies and for delivering therapeutic or suicide genes to cancers or tumours.

For use in human patients, the retroviral vectors should be replication defective. This prevents further generation of infectious retroviral particles in the target tissue-instead the replication defective vector becomes a “captive” transgene stable incorporated into the target cell genome. Typically in replication defective vectors, the gag, env, and pol genes have been deleted (along with most of the rest of the viral genome). Heterologous DNA is inserted in place of the deleted viral genes. The heterologous genes may be under the control of the endogenous heterologous promoter, another heterologous promoter active in the target cell, or the retroviral 5′ LTR (the viral LTR is active in diverse tissues). Typically, retroviral vectors have a transgene capacity of about 7-8 kb.

Replication defective retroviral vectors require provision of the viral proteins necessary for replication and assembly in trans, from, e.g., engineered packaging cell lines. It is important that the packaging cells do not release replication competent virus and/or helper virus. This has been achieved by expressing viral proteins from RNAs lacking the V signal, and expressing the gag/pol genes and the env gene from separate transcriptional units. In addition, in some 2. and 3. generation retriviruses, the 5′ LTR's have been replaced with non-viral promoters controlling the expression of these genes, and the 3′ promoter has been minimised to contain only the proximal promoter. These designs minimize the possibility of recombination leading to production of replication competent vectors, or helper viruses.

### X. Expression Vectors

Construction of vectors for recombinant expression of NsG29 and/or NsG31 polypeptides for use in the Invention may be accomplished using conventional techniques, which do not require detailed explanation to one of ordinary skill in the art. For review, however, those of ordinary skill may wish to consult Maniatis et al., In Molecular Cloning: A Laboratory Manual, Cold Spring Harbor Laboratory, (NY 1982). Expression vectors may be used for generating producer cells for recombinant production of NsG29 and/or NsG31 polypeptides for medical use, and for generating therapeutic cells secreting NsG29 and/or NsG31 polypeptides for naked or encapsulated therapy.

Briefly, construction of recombinant expression vectors employs standard ligation techniques. For analysis to confirm correct sequences in vectors constructed, the genes are sequenced using, for example, the method of Messing, et al., (Nucleic Acids Res., 9: 309-, 1981), the method of Maxam, et al., (Methods in Enzymology, 65: 499, 1980), or other suitable methods which will be known to those skilled in the art.

Size separation of cleaved fragments is performed using conventional gel electrophoresis as described, for example, by Maniatis, et al., (Molecular Cloning, pp. 133-134, 1982).

For generation of efficient expression vectors, these should contain regulatory sequences necessary for expression of the encoded gene in the correct reading frame. Expression of a gene is controlled at the transcription, translation or post-translation levels. Transcription initiation is an early and critical event in gene expression. This depends on the promoter and enhancer sequences and is Influenced by specific cellular factors that interact with these sequences. The transcriptional unit of many genes consists of the promoter and in some cases enhancer or regulator elements (Banerji et al., Cell 27: 299 (1981); Corden et al., Science 209: 1406 (1980); and Breathnach and Chambon, Ann. Rev. Biochem. 50: 349 (1981)). For retroviruses, control elements involved in the replication of the retroviral genome reside in the long terminal repeat (LTR) (Weiss et al., eds., The molecular biology of tumor viruses: RNA tumor viruses, Cold Spring Harbor Laboratory, (NY 1982)). Moloney murine leukemia virus (MLV) and Rous sarcoma virus (RSV) LTRs contain promoter and enhancer sequences (Jolly et al., Nucleic Acids Res. 11: 1855 (1983); Capecchi et al., In: Enhancer and eukaryotic gene expression, Gulzman and Shenk, eds., pp. 101-102, Cold Spring Harbor Laboratories (NY 1991). Other potent promoters include those derived from cytomegalovirus (CMV) and other wild-type viral promoters.

Promoter and enhancer regions of a number of non-viral promoters have also been described (Schmidt et al., Nature 314: 285 (1985); Rossi and deCrombrugghe, Proc. Natl. Acad. Sci. USA 84: 5590-5594 (1987)). Methods for maintaining and increasing expression of transgenes in quiescent cells include the use of promoters including collagen type I (1 and 2) (Prockop and Kivirikko, N. Eng. J. Med. 311: 376 (1984); Smith and Niles, Biochem. 19: 1820 (1980); de Wet et al., J. Biol. Chem., 258: 14385 (1983)), SV40 and LTR promoters.

According to one embodiment of the invention, the promoter is a constitutive promoter selected from the group consisting of: ubiquitin promoter, CMV promoter, JeT promoter (U.S. Pat. No. 6,555,674), SV40 promoter, and Elongation Factor 1 alpha promoter (EF1-alpha).

Examples of inducible/repressible promoters include: Tet-On, Tet-Off, Rapamycin-inducible promoter, Mx1.

In addition to using viral and non-viral promoters to drive transgene expression, an enhancer sequence may be used to increase the level of transgene expression. Enhancers can increase the transcriptional activity not only of their native gene but also of some foreign genes (Armelor, Proc. Natl. Acad. Sci. USA 70: 2702 (1973)). For example, in the present invention collagen enhancer sequences may be used with the collagen promoter 2 (I) to increase transgene expression. In addition, the enhancer element found in SV40 viruses may be used to increase transgene expression. This enhancer sequence consists of a 72 base pair repeat as described by Gruss et al., Proc. Natl. Acad. Sci. USA 78: 943 (1981); Benoist and Chambon, Nature 290: 304 (1981), and Fromm and Berg, J. Mol. Appl. Genetics, 1: 457 (1982), all of which are incorporated by reference herein. This repeat sequence can increase the transcription of many different viral and cellular genes when it is present in series with various promoters (Moreau et al., Nucleic Acids Res. 9: 6047 (1981).

Further expression enhancing sequences include but are not limited to Woodchuck hepatitis virus post-transcriptional regulation element, WPRE, SP163, CMV enhancer, and Chicken [beta]-globin insulator or other insulators.

Transgene expression may also be increased for long term stable expression using cytokines to modulate promoter activity. Several cytokines have been reported to modulate the expression of transgene from collagen 2 (I) and LTR promoters (Chua et al., connective Tissue Res., 25: 161-170 (1990); Elias et al., Annals N.Y. Acad. Sci., 580: 233-244 (1990)); Seliger et al., J. Immunol. 141: 2138-2144 (1988) and Seliger et al., J. Virology 62: 619-621 (1988)). For example, transforming growth factor (TGF), interleukin (IL)-I, and interferon (INF) down regulate the expression of transgenes driven by various promoters such as LTR. Tumor necrosis factor (TNF) and TGF 1 up regulate, and may be used to control, expression of transgenes driven by a promoter. Other cytokines that may prove useful include basic fibroblast growth factor (bFGF) and epidermal growth factor (EGF).

Collagen promoter with the collagen enhancer sequence (Coll (E)) may also be used to increase transgene expression by suppressing further any immune response to the vector which may be generated in a treated brain notwithstanding its immune-protected status. In addition, anti-inflammatory agents including steroids, for example dexamethasone, may be administered to the treated host immediately after vector composition delivery and continued, preferably, until any cytokine-mediated inflammatory response subsides. An immunosuppression agent such as cyclosporin may also be administered to reduce the production of interferons, which downregulates LTR promoter and Coll (E) promoter-enhancer, and reduces transgene expression.

The vector may comprise further sequences such as a sequence coding for the Cre-recombinase protein, and LoxP sequences. A further way of ensuring temporary expression of the NsG29 and/or NsG31 is through the use of the Cre-LoxP system which results in the excision of part of the inserted DNA sequence either upon administration of Cre-recombinase to the cells (Daewoong et al, Nature Biotechnology 19:929-933) or by incorporating a gene coding for the recombinase into the virus construct (Plück, Int J Exp Path, 77:269-278). Incorporating a gene for the recombinase in the virus construct together with the LoxP sites and a structural gene (an NsG29 and/or NsG31 in the present case) often results in expression of the structural gene for a period of approximately five days.

### XI. Biocompatible Capsules

Encapsulated cell therapy is based on the concept of isolating cells from the recipient host's immune system by surrounding the cells with a semipermeable biocompatible material before implantation within the host. The invention includes a device in which cells capable of expressing and secreting NsG29 and/or NsG31 are encapsulated in an immunoisolatory capsule. An “immunoisolatory capsule” means that the capsule, upon implantation into a recipient host, minimizes the deleterious effects of the host's immune system on the cells in the core of the device. Cells are immunoisolated from the host by enclosing them within implantable polymeric capsules formed by a microporous membrane. This approach prevents the cell-to cell contact between host and implanted tissues, eliminating antigen recognition through direct presentation. The membranes used can also be tailored to control the diffusion of molecules, such as antibody and complement, based on their molecular weight (Lysaght et al., 56 J. Cell Biochem. 196 (1996), Colton, 14 Trends Biotechnol. 158 (1996)). Using encapsulation techniques cells can be transplanted into a host without immune rejection, either with or without use of immunosuppressive drugs. Useful biocompatible polymer capsules usually contain a core that contains cells, either suspended in a liquid medium or immobilized within an immobilizing matrix, and a surrounding or peripheral region of permselective matrix or membrane (“jacket”) that does not contain isolated cells, that is biocompatible, and that is sufficient to protect cells in the core from detrimental immunological attack. Encapsulation hinders elements of the immune system from entering the capsule, thereby protecting the encapsulated cells from immune destruction. The semipermeable nature of the capsule membrane also permits the biologically active molecule of interest to easily diffuse from the capsule into the surrounding host tissue.

The capsule can be made from a biocompatible material. A “biocompatible material” is a material that, after implantation in a host, does not elicit a detrimental host response sufficient to result in the rejection of the capsule or to render it Inoperable, for example through degradation. The biocompatible material is relatively impermeable to large molecules, such as components of the host's immune system, but is permeable to small molecules, such as insulin, growth factors such as NsG29 and/or NsG31 polypeptides, and nutrients, while allowing metabolic waste to be removed. A variety of biocompatible materials are suitable for delivery of growth factors by the composition of the invention. Numerous biocompatible materials are known, having various outer surface morphologies and other mechanical and structural characteristics. Preferably the capsule of this invention will be similar to those described in WO 92/19195 or WO 95/05452, incorporated by reference; or U.S. Pat. Nos. 5,639,275; 5,653,975; 4,892,538; 5,156,844; 5,283,187; or U.S. Pat. No. 5,550,050, incorporated by reference. Such capsules allow for the passage of metabolites, nutrients and therapeutic substances while minimizing the detrimental effects of the host immune system. Components of the biocompatible material may include a surrounding semipermeable membrane and the internal cell-supporting scaffolding. Preferably, the genetically altered cells are seeded onto the scaffolding, which is encapsulated by the permselective membrane. The filamentous cell-supporting scaffold may be made from any biocompatible material selected from the group consisting of acrylic, polyester, polyethylene, polypropylene polyacetonitile, polyethylene teraphthalate, nylon, polyamides, polyurethanes, polybutester, silk, cotton, chitin, carbon, or biocompatible metals. Also, bonded fiber structures can be used for cell implantation (U.S. Pat. No. 5,512,600, incorporated by reference). Biodegradable polymers include those comprised of poly(lactic acid) PLA, poly(lactic-coglycolic acid) PLGA, and poly(glycolic acid) PGA and their equivalents. Foam scaffolds have been used to provide surfaces onto which transplanted cells may adhere (WO 98/05304, incorporated by reference). Woven mesh tubes have been used as vascular grafts (WO 99/52573, incorporated by reference). Additionally, the core can be composed of an immobilizing matrix formed from a hydrogel, which stabilizes the position of the cells. A hydrogel is a 3-dimensional network of cross-linked hydrophilic polymers in the form of a gel, substantially composed of water.

Various polymers and polymer blends can be used to manufacture the surrounding semipermeable membrane, including polyacrylates (including acrylic copolymers), polyvinylidenes, polyvinyl chloride copolymers, polyurethanes, polystyrenes, polyamides, cellulose acetates, cellulose nitrates, polysulfones (including polyether sulfones), polyphosphazenes, polyacrylonitriles, poly(acrylonitrile/covinyl chloride), as well as derivatives, copolymers and mixtures thereof. Preferably, the surrounding semipermeable membrane is a biocompatible semipermeable hollow fiber membrane. Such membranes, and methods of making them are disclosed by U.S. Pat. Nos. 5,284,761 and 5,158,881, incorporated by reference. The surrounding semipermeable membrane is formed from a polyether sulfone hollow fiber, such as those described by U.S. Pat. No. 4,976,859 or U.S. Pat. No. 4,968,733, incorporated by reference. An alternate surrounding semipermeable membrane material is poly(acrylonitrile/covinyl chloride).

The capsule can be any configuration appropriate for maintaining biological activity and providing access for delivery of the product or function, including for example, cylindrical, rectangular, disk-shaped, patch-shaped, ovoid, stellate, or spherical. Moreover, the capsule can be coiled or wrapped into a mesh-like or nested structure. If the capsule is to be retrieved after it is implanted, configurations which tend to lead to migration of the capsules from the site of implantation, such as spherical capsules small enough to travel in the recipient host's blood vessels, are not preferred. Certain shapes, such as rectangles, patches, disks, cylinders, and flat sheets offer greater structural integrity and are preferable where retrieval is desired.

When macrocapsules are used, preferably between 10³ and 10⁸ cells are encapsulated, most preferably 10⁵ to 10⁷ cells are encapsulated in each device. Dosage may be controlled by implanting a fewer or greater number of capsules, preferably between 1 and 10 capsules per patient.

The scaffolding may be coated with extracellular matrix (ECM) molecules. Suitable examples of extracellular matrix molecules include, for example, collagen, laminin, and fibronectin. The surface of the scaffolding may also be modified by treating with plasma irradiation to impart charge to enhance adhesion of cells.

Any suitable method of sealing the capsules may be used, including the use of polymer adhesives or crimping, knotting and heat sealing. In addition, any suitable “dry” sealing method can also be used, as described, e.g., in U.S. Pat. No. 5,653,687, incorporated by reference.

The encapsulated cell devices are implanted according to known techniques. Many implantation sites are contemplated for the devices and methods of this invention. These implantation sites include, but are not limited to, the central nervous system, including the brain, spinal cord (see, U.S. Pat. Nos. 5,106,627, 5,156,844, and 5,554,148, incorporated by reference), and the aqueous and vitreous humors of the eye (see, WO 97/34586, incorporated by reference).

Methods and apparatus for implantation of capsules into the CNS are described in U.S. Pat. No. 5,487,739. Methods and apparatus for implantation of capsules into the eye are described in U.S. Pat. No. 5,904,144, U.S. Pat. No. 6,299,895, U.S. Pat. No. 6,439,427, and US 20030031700.

In one aspect the invention relates to a biocompatible capsule comprising: a core comprising living packaging cells that secrete a viral vector for infection of a target cell, wherein the viral vector is a vector according to the invention; and an external jacket surrounding said core, said jacket comprising a permeable biocompatible material, said material having a porosity selected to permit passage of retroviral vectors of approximately 100 nm diameter thereacross, permitting release of said viral vector from said capsule.

Preferably, the core additionally comprises a matrix, the packaging cells being immobilized by the matrix. According to one embodiment, the jacket comprises a hydrogel or thermoplastic material.

Examples of suitable cells for packaging cell lines include HEK293, NIH3T3, PG13, and ARPE-19 cells. Preferred cells include PG13 and 3T3 cells.

Packaging cell lines may be encapsulated and administered using the methods and compositions disclosed in U.S. Pat. No. 6,027,721 and WO 97/01357 hereby incorporated by reference in their entirety.

### XII Support Matrix for NsG29 and/or NsG31 Producing Cells

The present invention further comprises culturing NsG29 and/or NsG31 producing cells in vitro on a support matrix prior to implantation into the mammalian nervous system. The preadhesion of cells to microcarriers prior to implantation is designed to enhance the long-term viability of the transplanted cells and provide long term functional benefit.

To increase the long term viability of the transplanted cells, i.e., transplanted NsG29 and/or NsG31 secreting cells, the cells to be transplanted can be attached in vitro to a support matrix prior to transplantation. Materials of which the support matrix can be comprised include those materials to which cells adhere following in vitro incubation, and on which cells can grow, and which can be implanted into the mammalian body without producing a toxic reaction, or an inflammatory reaction which would destroy the implanted cells or otherwise interfere with their biological or therapeutic activity. Such materials may be synthetic or natural chemical substances, or substances having a biological origin.

The matrix materials include, but are not limited to, glass and other silicon oxides, polystyrene, polypropylene, polyethylene, polyvinylidene fluoride, polyurethane, polyalginate, polysulphone, polyvinyl alcohol, acrylonitrile polymers, polyacrylamide, polycarbonate, polypentent, nylon, amylases, natural and modified gelatin and natural and codified collagen, natural and modified polysaccharides, including dextrans and celluloses (e.g., nitrocellulose), agar, and magnetite. Either resorbable or non-resorbable materials may be used. Also intended are extracellular matrix materials, which are well-known in the art. Extracellular matrix materials may be obtained commercially or prepared by growing cells which secrete such a matrix, removing the secreting cells, and allowing the cells which are to be transplanted to interact with and adhere to the matrix. The matrix material on which the cells to be implanted grow, or with which the cells are mixed, may be an indigenous product of RPE cells. Thus, for example, the matrix material may be extracellular matrix or basement membrane material, which is produced and secreted by RPE cells to be implanted.

To improve cell adhesion, survival and function, the solid matrix may optionally be coated on its external surface with factors known in the art to promote cell adhesion, growth or survival. Such factors include cell adhesion molecules, extracellular matrix, such as, for example, fibronectin, laminin, collagen, elastin, glycosaminoglycans, or proteoglycans or growth factors.

Alternatively, if the solid matrix to which the implanted cells are attached is constructed of porous material, the growth- or survival promoting factor or factors may be incorporated into the matrix material, from which they would be slowly released after implantation in vivo.

When attached to the support according to the present invention, the cells used for transplantation are generally on the “outer surface” of the support. The support may be solid or porous. However, even in a porous support, the cells are in direct contact with the external milieu without an intervening membrane or other barrier. Thus, according to the present invention, the cells are considered to be on the “outer surface” of the support even though the surface to which they adhere may be in the form of internal folds or convolutions of the porous support material which are not at the exterior of the particle or bead itself.

The configuration of the support is preferably spherical, as in a bead, but may be cylindrical, elliptical, a flat sheet or strip, a needle or pin shape, and the like. A preferred form of support matrix is a glass bead. Another preferred bead is a polystyrene bead.

Bead sizes may range from about 10 μm to 1 mm in diameter, preferably from about 90 μm to about 150 μm. For a description of various microcarrier beads, see, for example, isher Biotech Source 87-88, Fisher Scientific Co., 1987, pp. 72-75; Sigma Cell Culture Catalog, Sigma Chemical Co., St, Louis, 1991, pp. 162-163; Ventrex Product Catalog, Ventrex Laboratories, 1989; these references are hereby incorporated by reference. The upper limit of the bead's size may be dictated by the bead's stimulation of undesired host reactions, which may interfere with the function of the transplanted cells or cause damage to the surrounding tissue. The upper limit of the bead's size may also be dictated by the method of administration. Such limitations are readily determinable by one of skill in the art.

### XIII. Host Cells

In one aspect the invention relates to isolated host cells genetically modified with the vector according to the invention.

According to one embodiment, the host cells are prokaryotic cells such as E. coli which are capable producing recombinant protein in high quantities and which can easily be scaled up to industrial scale. The use of prokaryotic producer cells may require refolding and glycosylation of the NsG29 and/or NsG31 in order to obtain a biologically active protein. In another embodiment, the host cells are eukaryotic producer cells from non-mammals, including but not limited to known producer cells such as yeast (Saccharomyces cerevisiae), filamentous fungi such as aspergillus, and insect cells, such as Sf9.

According to another embodiment, the cells preferably are mammalian host cells because these are capable of secreting and processing the encoded NsG29 and/or NsG31 correctly. Preferred species include the group consisting of human, feline, porcine, simian, canina, murine, rat, rabbit, mouse, and hamster.

Examples of primary cultures and cell lines that are good candidates for transduction or transfection with the vectors of the present invention include the group consisting of CHO, CHO-K1, HEI193T, HEK293, COS, PC12, HiB5, RN33b, neuronal cells, foetal cells, ARPE-19, C2C12, HeLa, HepG2, striatal cells, neurons, astrocytes, and interneurons. Preferred cell lines for mammalian recombinant production include CHO, CHO-1, HEI193T, HEK293, COS, PC12, HiB5, RN33b, and BHK cells.

For ex vivo gene therapy, the preferred group of cells include neuronal cells, neuronal precursor cells, neuronal progenitor cells, stem cells and foetal cells.

The invention also relates to cells suitable for biodelivery of NsG29 and/or NsG31 via naked or encapsulated cells, which are genetically modified to overexpress NsG29 and/or NsG31, and which can be transplanted to the patient to deliver bioactive NsG29 and/or NsG31 polypeptide locally. Such cells may broadly be referred to as therapeutic cells.

In a preferred embodiment of the invention, a therapeutic cell line has not been immortalised with the insertion of a heterologous immortalisation gene. As the invention relates to cells which are particularly suited for cell transplantation, whether as naked cells or—preferably as encapsulated cells, such immortalised cell lines are less preferred as there is an Inherent risk that they start proliferating in an uncontrolled manner inside the human body and potentially form tumours.

Preferably, the cell line is a contact inhibited cell line. By a contact inhibited cell line is intended a cell line which when cultured in Petri-dishes grow to confluency and then substantially stop dividing. This does not exclude the possibility that a limited number of cells escape the monolayer. Contact inhibited cells may also be grown in 3D, e.g. inside a capsule. Also inside the capsules, the cells grow to confluency and then significantly slow down proliferation rate or completely stop dividing. A particularly preferred type of cells includes epithelial cells which are by their nature contact-inhibited and which form stable monolayers in culture.

Even more preferred are retinal pigment epithelial cells (RPE cells). The source of RPE cells is by primary cell isolation from the mammalian retina. Protocols for harvesting RPE cells are well-defined (Li and Turner, 1988, Exp. Eye Res. 47:911-917; Lopez et al., 1989, Invest. Opthalmol. Vis. Sci. 30:586-588) and considered a routine methodology. In most of the published reports of RPE cell cotransplantation, cells are derived from the rat (Li and Turner, 1988; Lopez et al., 1989). According to the present invention RPE cells are derived from humans. In addition to isolated primary RPE cells, cultured human RPE cell lines may be used in the practice of the invention.

For encapsulation, the cells need to be able to survive and maintain a functional NsG29 and/or NsG31 secretion at the low oxygen tension levels of the CNS. Preferably the cell line of the invention is capable of surviving at an oxygen tension below 5%, more preferably below 2%, more preferably below 1%. 1% oxygen tension corresponds approximately to the oxygen level in the brain.

To be a platform cell line for an encapsulated cell based delivery system, the cell line should have as many of the following characteristics as possible: (1) The cells should be hardy under stringent conditions (the encapsulated cells should be functional in the vascular and avascular tissue cavities such as in the central nervous system intraparenchymally or within the ventricular or intrathecal fluid spaces or the eye, especially in the intra-ocular environment). (2) The cells should be able to be genetically modified to express NsG29 and/or NsG31. (3) The cells should have a relatively long life span (the cells should produce sufficient progenies to be banked, characterised, engineered, safety tested and clinical lot manufactured). (4) The cells should be of human origin (which increases compatibility between the encapsulated cells and the host). (5) The cells should exhibit greater than 80% viability for a period of more than one month in vivo in device (which ensures long-term delivery). (6) The encapsulated cells should deliver an efficacious quantity of NsG29 and/or NsG31 (which ensures effectiveness of the treatment). (7) when encapsulated the cells should not cause a significant host immune reaction (which ensures the longevity of the graft). (8) The cells should be non-tumourigenic (to provide added safety to the host, in case of device leakage).

For encapsulation the preferred cells include retinal pigmented epithelial cells, including ARPE-19 cells; human immortalised fibroblasts; and human immortalised astrocytes.

The ARPE-19 cell line is a superior platform cell line for encapsulated cell based delivery technology and is also useful for unencapsulated cell based delivery technology. The ARPE-19 cell line is hardy (i.e., the cell line is viable under stringent conditions, such as implantation in the central nervous system or the intra-ocular environment). ARPE-19 cells can be genetically modified to secrete a substance of therapeutic interest. ARPE-19 cells have a relatively long life span. ARPE-19 cells are of human origin. Furthermore, encapsulated ARPE-19 cells have good in vivo device viability. ARPE-19 cells can deliver an efficacious quantity of growth factor. ARPE-19 cells elicit a negligible host immune reaction. Moreover, ARPE-19 cells are non-tumorigenic. Methods for culture and encapsulation of ARPE-19 cells are described in U.S. Pat. No. 6,361,771.

In another embodiment the therapeutic cell line is selected from the group consisting of: human fibroblast cell lines, human astrocyte cell lines, human mesencephalic cell lines, and human endothelial cell lines, preferably immortalised with TERT, SV40T or vmyc.

The method for generating an immortalised human astrocyte cell lines has previously been described (Price T N, Burke J F, Mayne L V. A novel human astrocyte cell line (A735) with astrocyte-specific neurotransmitter function. In Vitro Cell Dev Biol Anim. 1999 May; 35(5):279-88). This protocol may be used to generate astrocyte cell lines.

The following three modifications of that protocol are preferably made to generate additional human astrocyte cell lines.

Human foetal brain tissue dissected from 5-12 weeks old foetuses may be used instead of 12-16 weeks old tissue.

The immortalisation gene v-myc, or TERT (telomerase) may be used instead of the SV40 T antigen.

Retroviral gene transfer may be used instead of transfection with plasmids by the calcium phosphate precipitation technique.

### EXAMPLES

The following examples are offered by way of illustration and not by way of limitation. It will be understood that the specific conditions of the examples can be varied without departing from the purpose of the examples.

### Example 1a

### NsG31 Sequences

SEQ ID NO 1, human NsG31 genomic sequence with 100 extra basepairs added in the ends of 5′ and 3′.
SEQ ID NO 2, human NsG31 cDNA
SEQ ID NO 3, human NsG31 full length amino acid sequence
SEQ ID No. 4 human NsG31, mature protein
SEQ ID No. 5, human NsG31, Cys1-Cys10 polypeptide fragment
SEQ ID No. 6, human NsG31, core polypeptide fragment
SEQ ID NO 7, Mouse NsG31 genomic nucleotide sequence with 100 extra basepairs added in the 5′ end and 100 extra basepairs added in the 3′ end.
SEQ ID NO 8, mouse NsG31 cDNA
SEQ ID NO 9, mouse NsG31 full length amino acid sequence
SEQ ID No. 10, mouse NsG31 mature protein
SEQ ID no. 11, mouse NsG31, Cys1-Cys10 polypeptide fragment
SEQ ID no. 12, mouse NsG31, core polypeptide fragment
SEQ ID NO 13, human NsG31-long genomic sequence with 100 extra basepairs added in the ends of 5′ and 3′.
SEQ ID NO 14, human NsG31-long cDNA
SEQ ID NO 15, human NsG31-long full length amino acid sequence
SEQ ID No. 16, human NsG31-long mature protein

| Human NsG31 genomic nucleotide sequence (exons in CAPS) | |
|---|---|
| (SEQ ID NO 1) | |
| ccaccccctt gccctctccc aggttctcct tcaccccctg cagagccatc | 50 |
| ggctccctct atcctttgca accccctatg aatctttcct acttgtccag | 100 |
| GTGGAGTGAG TCTGAGGACA GCAGATGAAC AGACAGAAAC TGAAAGATCC | 150 |
| CCAAAAAGgt aagtggggag acattgaaga tggggagtag cgggggtgga | 200 |
| cagagatcaa gtaccctatc tgaggagcaa ctgggtccta gcagttggtt | 250 |
| tggcctctgg tattcccaaa gctgcactgg cacttgtggg ggcccagcag | 300 |
| tggtccaggc tcacccctga gcttttggcc ttgctgcatc ctgggacttc | 350 |
| tcctgctccc ttcaatggct gggcagctcc tagctactct gtgcctcttg | 400 |
| cttccttagg attccctcca tttccagcct acccttcagg gtagctttgg | 450 |
| gaggcccaaa ccatcaacga tgaaagagga aaaggaaagg ggaagagaac | 500 |
| agaggcaagg tggtgcagtg ggcagagggc aggcttctag gctctgaact | 550 |
| cttccaccca catgctgcct ggccttgggc aagtcatttg acttctgtta | 600 |
| gcctcggttt cacctcttcc tttttaaatt taaattctgt ttaaattgaa | 650 |
| gtgctactgc attcccgtgg ttggcagctc aaaaagcaag aaataaatca | 700 |
| ggagaaaaag tctctcttct acctccatct cccaaccaac cagtttccct | 750 |
| tttggatggc aaacaatatt actagtttta aaatatattt ttcaaagcta | 800 |
| gtttatgcat atctgagcaa catagataat ccttccttta tacacatttt | 850 |
| attttattta tttttgagct agggtctcac tctgttgccc aggctggagt | 900 |
| gcagtagcac agtcatagat cactgcagcc tcaaactcct gatcctctca | 950 |
| cctcagcctc tggagtagct gggactatag gtgtgcacca ccatgcctga | 1000 |
| ctaatttttt aattttttgt agagatgggg tcttgctatg ttgcctaggc | 1050 |
| tggtctcgaa cttctggcct caagtgatcc tcctgccttg gcctcccaaa | 1100 |
| gtgctggaat tagaggcatg aaccaccact cctggcctgc cttatacaca | 1150 |
| tcgtatacac accattctac actttgcttt ttttccccct taacagtgga | 1200 |
| ttatcactgg agatcactgc atgtgtcagg acataaagca attccccatt | 1250 |
| ggttttttta cagctatctg gatgggactg catttgtaca gactctttac | 1300 |
| gatgtattca gccagtcttt tactgatggt tgtttaggtt atgtcctagc | 1350 |
| tttttgagct ttggttttct catctgtaaa acaggtaata ataatgttgc | 1400 |
| tttgtaggac agttgtgaag aattcaagag tgtgcatgtg aagtgctgga | 1450 |
| accaagcact caaagagtag gagtcagtat tattgtccct cccatgtcac | 1500 |
| gaccctccat gggcccatag ctagttcatc ctccaagatg cctcttaaat | 1550 |
| gttgtctcct ccaaaggctc tccttttctt cctgcagcac tcactactgc | 1600 |
| ctgcatgact ctgtgccagg tcgaaagatg cctggggaca aggtgtgcct | 1650 |
| cctccccatc tttgtgcctc ccagagtgtg tggcacagag aaggtgccca | 1700 |
| ggtgcacagg ggagcttctg cctggagctg agcagaatgt gtgcttctct | 1750 |
| ccgcagGATG AGTGAGAGGG TCGAGCGGAA CTGGAGCACG GGCGGCTGGC | 1800 |
| TGCTGGCACT GTGCCTGGCC TGGCTGTGGA CCCACCTGAC CTTGGCTGCC | 1850 |
| tTGCAGCCTC CCACTGCCAC AGgtttggag gaggtggcag ggcccgggga | 1900 |
| gggagtttcc caggacacct ggggagctgg acaccactcc accgtgttcc | 1950 |
| atagcaggca ggtctcccag gcagagtaaa atccagagat tctaggggtt | 2000 |
| ccggctcctc ctctgctctt tacccaggaa tctctgccca ccccagctgt | 2050 |
| cccgccccag agaaggtctg ggcagaccct ttgctcacct ctccttccag | 2100 |
| gctggaaggt gttctgtagt gagagaggcc ctcgggtgag ggcttatctc | 2150 |
| tagcctcctg gcctaattct atggttttcc ttctgggatg gctaaagcct | 2200 |
| gtgtaagcgg atgatcctcc ccacgcattg taagccctgg ggcccttgcc | 2250 |
| ctcaccctga ggaagctggg gaggcggggc tggtgcggga ctctgtcttc | 2300 |
| tgcccagggg ccgtgctctg ccaaagggtg aagagcacag gcctcacagg | 2350 |
| ggagggagga agaataggag ctctgtgaag cggcgggaag atttcatcca | 2400 |
| cttcctcctc ccctgccact ggcagtctct tccatagccc tccctgcagc | 2450 |
| agggaggagg ggctggaggc cagcccgggt gggcgggagg gagcggggtc | 2500 |
| gggcgtgttg ggcgtctgat cctgggcggc tcccctcagT GCTTGTGCAG | 2550 |
| CAGGGCACCT GCGAGGTGAT TGCGGCTCAC CGCTGCTGCA ACCGGAACCG | 2600 |
| CATCGAGGAG CGCTCCCAGA CGGTGAAATG CTCCTGTTTT TCTGGCCAGG | 2650 |
| TGGCCGGCAC CACGCGGGCA AAGCCCTCCT GCGTGGACGg tgagtgagag | 2700 |
| gccaggaccc gggcagggcc ctgagcagag ccataaggag gcctgttcag | 2750 |
| gcctcgccat ctgctgggga tacaatttca tacccgtctc agggctttca | 2800 |
| tgagcagtct caaatggtgg ggccccctct gggagaagag ggcagacgtg | 2850 |
| gccatccctg tgtcatagat gagaaaagct cttgttatgc gcacagcctc | 2900 |
| ccacactcac gtgccctctg cctgttgcac acacaggagc gttccctcac | 2950 |
| acagccccac tcctctcccc acacacttcc tccagctcct tcccctgctg | 3000 |
| ctccttcccc tggctgtctc agcctactct cctctccttc tcagcccttc | 3050 |
| tctcacactt tcctatgtgg acacgtgtga tccttcacac ccacactcat | 3100 |
| taccctaaca ctcatttggc accccataca catgcccact gtgtcacgcg | 3150 |
| cataccagct atgccagcta tgcactcctg gtctgtagac ccctccagac | 3200 |
| gactcacacc cttgctcaca caccacagag ggagtatact ggttgtgcat | 3250 |
| cgatgctagt gacatggtca ccttagagac atttttgcct gccttcaccc | 3300 |
| gtcactgtct ggggagatgc agggatcggt gggattaaag ttgagagggt | 3350 |
| tacgtcccca ggcctcaggc tgagtactgc ctctctgggc agcctggagt | 3400 |
| aggggaaaga caagggaaat gcatggctca aggccagggg tgtgtggaaa | 3450 |
| cgtgcacctc attcgcagac ctgctcttgg ctgcccactg tgctcgtaga | 3500 |
| gaccctagag ctgcactccg cctcctgctc ccacagCCTC CATCGTCCTG | 3550 |
| CAGAGATGGT GGTGTCAGAT GGAGCCCTGC CTGCCGGGGG AGGAGTGTAA | 3600 |
| GGTGCTCCCG GACCTGTCGG GATGGAGCTG CAGCAGTGGA CACAAAGTCA | 3650 |
| AAACCACCAA Ggtaccctgg gtgggcacct catcccaccc tccccttccc | 3700 |
| tctctaccaa gggcagaaaa gggggcacag gagccttgga ggtctaggat | 3750 |
| gtcagtgtcc caaggtgaga tcgtgaacca tgaaattcac agacagtcct | 3800 |
| ctcaggtctg ttctcactgg ggactggagt taggggggtc aggacttgaa | 3850 |
| atccagtgtg tcctgggaag attcttgatg tccatatacg ctggtttcct | 3900 |
| cctgtctgcc tggaattagt ttgcctccag aaaatatcag gcctataaaa | 3950 |
| acatagtcca tggaatacta tgcagccata aaaaatgatg agttcatgtc | 4000 |
| ctttgtaggg acatggatga aattggaaat catcattctc agtaaactat | 4050 |
| cgcaagaaca aaaaaccaaa caccgcatat tctcactcat aggtgggaac | 4100 |
| tgaacaatga gatcacatgg acacaggaag gggaatatca cactctgggg | 4150 |
| actgtggtgg ggtgggggga ggggggaggg gtagcattgg gagatatacc | 4200 |
| taatgctaga tgacgagtta gtgggtgcag cgcaccagca tggcacatgt | 4250 |
| atacatatgt aactaacctg cacaatgtgc acatgtaccc taaaacgtaa | 4300 |
| agtataattt aaaaaaaaaa aaacaacata ttagagcagc aaaaaaaaaa | 4350 |
| aaaaaaaaaa aaaagagctt aaaacagaac taccattcaa cccagccatc | 4400 |
| ctattactgg tatataccca aaggaaaata aatcattttg ccaaaaagac | 4450 |
| acatgcactt gtatgttcat caaagcagta ttcataatag caagggcata | 4500 |
| taatcagtct aggtgccaat caacattgaa taggataaag aaaatgtggc | 4550 |
| acttatatac catagaatac tacacagcca taaaaaaaat aatgttttct | 4600 |
| gtaacaccct ggttgcagct gaaggccatg atcctaaggg acttagcatg | 4650 |
| gcaacggaaa atcaaatacc acatgttctc acttctgaga gctaaacatt | 4700 |
| gggtacacat ggacataaag atgggaacag tggacgatgg tgactaccag | 4750 |
| agcagtgaga gagggagggg agcaagggct gaaaaactac ctattgggtc | 4800 |
| ctatgcttac tacatgggta atgagatcaa tggcactcca aacctcagtg | 4850 |
| tcatgcaata tactcatgta acaaacctgc acatgtaatc cctgaatcta | 4900 |
| aaataaaagt tgaaatatta aaaaaaaaaa aaaaaaaaac atagtcgaga | 4950 |
| atgggaaaaa ttataagata attgagggag gaattgctac ttctacctga | 5000 |
| gctccaggta cagtagtcac tctgtcactc acctctgccg tgaccatccc | 5050 |
| ccacccccac cccattccca ctgttgttga ctccctacag ttcataccta | 5100 |
| ccctcctcta gtcagatttc tctgtccact gtcacagcac ccaagaggat | 5150 |
| tattattttc atggtttgct gcctgctcca cagaacaagg ggtaattaaa | 5200 |
| ttttttatca aactcgttag cagctccatg tgtgtcatgg ctaatcagcc | 5250 |
| gcattgctct gccgtcacca ttggtacaga agagtgacac tgcagggcaa | 5300 |
| gccagggttc tgcctttggc tttccttccc tcccaccaca cacagcaaac | 5350 |
| aagcaaacac ataagaaaat agagaaggag ggctgggtgc ggtggctcat | 5400 |
| gcctgtaatc ccagcacttt gggagacaga ggtgggtgaa tcaagaggtc | 5450 |
| aggagttcaa gaccaacctg gtcaacatgg tgaaaacccg tctctactaa | 5500 |
| aaatacaaaa attagctggg cataatggca cacgcctgta atcccagcta | 5500 |
| cttgggaggc tgaggcagga gaattgcttg aacctaggag gcagaagttg | 5600 |
| cggtaagcag agatcatgcc actgcactcc agtctgggcg acagagtgag | 5650 |
| actccatctc aaaaaaagaa aaaaagaaaa aaaaatagag aaggaaaggc | 5700 |
| agcctttggc ttctgaagag agcaaccatt ttcacagagc acacaacaaa | 5750 |
| aggagctaca gcaagaaaaa gtttggctct aaagtagagc atcctctctc | 5800 |
| tatccggagg cagggatgag ggacagccat tctgagtata tttaagatca | 5850 |
| gaagagaatg agacccacta cagagcttgt gacatttgtc accatgttat | 5900 |
| aggtttgggt ttgtggcctg taaggaagtg cttgcaattt gggtagctac | 5950 |
| tctgaagcaa acagtgggga ggcttgtctg acgtatgttt gcatgggtca | 6000 |
| ccaggccatg ctttcccatt ccggcctgct ttagggtgag aggattggca | 6050 |
| gcaatgtcct ctaagtgacc tgcatattct gggtagtctc tggcatgctg | 6100 |
| gaatggaata ggcattccct tctctaacct taccctctcg cttcttcacc | 6150 |
| agGTCACACG ATAGCTCTTG GGGGTCACGG CCTGGACAAG AAAGGCTTGA | 6200 |
| CTGAGCCGTG AACTGAAGAA TGGTATCCAG TCATCAGCCA GGAAAGATGG | 6250 |
| GGATTCACTT ACATGCCTCA TGTCAAATGC AGCATCAGTC TTTCCGGGGC | 6300 |
| ATTTCAGTTA AGCTGCTCAG CAGATATGGA TGGATCTGCA ATCACATACC | 6350 |
| TAATGTGGAG CTGGGCTTTT CTGGAGACAC GAAGGTCAAC ACACAATTCC | 6400 |
| TGCCCTTAAG GAATGTCCAG TTGAATTGGA GAGTTGATGA CAGACAATTT | 6450 |
| AGATAATTTA GGTTAAAGTA CTTGATACCA GACTGCGGCT TCTGGGCCAC | 6500 |
| ATGCTATGGC ATGATGGGGG TTTGGGAATG AGATTCCCAC AGTTCTTCAG | 6550 |
| ATACCCTGTG GCCACAGGGC ATAGAAACAA GAGGTCACAT TCAGCACCCA | 6600 |
| CCACCTCCCT CTTTCGCATC AGTTCTGAAT CCCCAGCAAG CTGTTAACAT | 6650 |
| GTTGCAGGAA AACACTCTCC CCTTATtCCA GACCAGCAGT ATCTCATTTG | 6700 |
| GATGGGATTG GATTGACTTG CGGAAGGAAA GTAAAAATAA AGCCAAATAA | 6750 |
| CTTCCCAAAt ctttggcaca ggtatgggcc attcattggg tatgggatgg | 6800 |
| acaggacttt aaaatcatct ccatccaagc acctcaggtt tttgtttgtt | 6850 |
| tgcttattt | 6859 |
| Human NsG31 (1077 bp; CDS = 70-471) | |

(SEQ ID NO 2)
>gi|32967230|gb|AY325116.1|Homo sapiens TAFA3 mRNA, complete
cds
GGTGGAATTCGTGGAGTGAGTCTGAGGACAGCAGATGAACAGACAGAAACTGAAAGATCC
CCAAAAAGGATGAGTGAGAGGGTCGAGCGGAACTGGAGCACGGGCGGCTGGCTGCTGGCA
CTGTGCCTGGCCTGGCTGTGGACCCACCTGACCTTGGCTGCCCTGCAGCCTCCCACTGCC
ACAGTGCTTGTGCAGCAGGGCACCTGCGAGGTGATTGCGGCTCACCGCTGCTGCAACCGG
AACCGCATCGAGGAGCGCTCCCAGACGGTGAAATGCTCCTGTTTTTCTGGCCAGGTGGCC
GGCACCACGCGGGCAAAGCCCTCCTGCGTGGACGCCTCCATCGTCCTGCAGAGATGGTGG
TGTCAGATGGAGCCCTGCCTGCCGGGGGAGGAGTGTAAGGTGCTCCCGGACCTGTCGGGA
TGGAGCTGCAGCAGTGGACACAAAGTCAAAACCACCAAGGTCACACGATAGCTCTTGGGG
GTCACGGCCTGGACAAGAAAGGCTTGACTGAGCCGTGAACTGAAGAATGGTATCCAGTCA
TCAGCCAGGAAAGATGGGGATTCACTTACATGCCTCATGTCAAATGCAGCATCAGTCTTT
CCGGGGCATTTCAGTTAAGCTGCTCAGCAGATATGGATGGATCTGCAATCACATACCTAA
TGTGGAGCTGGGCTTTTCTGGAGACACGAAGGTCAACACACAATTCCTGCCCTTAAGGAA
TGTCCAGTTGAATTGGAGAGTTGATGACAGAcAATTTAGATAATTTAGGTTAAAGTACTT
GATACCAGACTGCGGCTTCTGGGCCACATGCTATGGCATGATGGGGGTTTGGGAATGAGA
TTCCCACAGTTCTTCAGATACCCTGTGGCCACAOGGCATAGAAACAAGAGGTCACATTCA
GCACCCACCACCTCCCTCTTTCGCATCAGTTCTGAATCCCCAGCAAGCTGTTAACATGTT
GCAGGAAAACACTCTCCCCTTATGCCAGACCAGCAGTATCTCATTTGGATGGGATTGGAT
TGACTTGCGGAAGGAAAGTAAAAATAAAGCCAAATAACTTCCCAAAAAAAAAAAAAA
Human NsG31 full length amino acid sequence
(SEQ ID NO 3)
>IPI00334480.1 REFSEQ_NP:NP_877436 ENSEMBL:ENSP00000333581
Tax_Id = 9606 TAFA3
MSERVERNWS TGGWLLALCL AWLWTHLTLA ALQPPTATVL VQQGTCEVIA
AHRCCNRNRI EERSQTVKCS CFSGQVAGTT RAKPSCVDAS IVLQRWWCQM
EPCLPGEECK VLPDLSGWSC SSGHKVKTTK VTR
Human NsG31 mature protein
(SEQ ID NO 4)
ALQPPTATVL VQQGTCEVIA AHRCCNRNRI EERSQTVKCS CFSGQVAGTT
RAKPSCVDAS IVLQRWWCQM EPCLPGEECK VLPDLSGWSC SSGHKVKTTK
VTR
Human NsG31 Cys1-Cys10 fragment
(SEQ ID NO 5)
CEVIAAHRCC NRNRIEERSQ TVKCSCFSGQ VAGTTRAKPS CVDASIVLQR
WWCQMEPCLP GEECKVLPDL SGWSC
Human NsG31 core polypeptide fragment
(SEQ ID NO 6)
GTCEVIAAHR CCNRNRIEER SQTVRCSCFS GQVAGTTRAK PSCVDASIVL
QRWWCQMEPC LPGEECKVLP DLSGWSCSSG HKVKTTK

Mouse NsG31 Genomic Nucleotide Sequence with 100 Extra Basepairs Added in the 6 and 3′ Ends.

| (SEQ ID NO 7) | |
|---|---|
| Genomic chr3 (reverse strand); exons in CAPS: | |
| aatggctgcc tttgccttgg gacagcctaa gctggagttc ctcatcctcc | 50 |
| ctttctctta ctctcttcca agcttcctct tagcccctcc aggggtctgg | 100 |
| TCTCTCTCCA TTCTGAACAA CCGCCATATT GTTCATCAGA tGGAACAAGT | 150 |
| CTGAGACCAG CAGATGAACA GGCAGAGGCC TGAAGACCCC AGGAACAgta | 200 |
| agtgagacat ctggagatga gaattgagga gaagactgcc cctccaaggg | 250 |
| agcagctgtt tccttctttt tggcccctgg tattcctaga actgtgctgt | 300 |
| caattgctgg ggacagtgac caggctaacc cgtggcctcc tggggctctt | 350 |
| cctgtcccct tctgtggcta gtttctctca gcttcttttt gttgctactt | 400 |
| ttcgggtttg gcctctggtt cttacccact tctggtacag atgttggagg | 450 |
| tcttgcttgg ttcatagaag acaggaggaa gagaaagcct caggcaggct | 500 |
| tctgggtcct gacccatgac cctggccctt gggcaaagct ttcaacttct | 550 |
| aatgggggag gaggtggtct tccttccttc cttccttcct tccttccttc | 600 |
| cttccttcct tccttccttc tttccttcct tctttccttc cttccttcct | 650 |
| tccttccttc cttccttcct tctttccttc cttccttctt tccttccttc | 700 |
| cttccttcct tctttccttc cttccttcct tcctttcttc cttccttcct | 750 |
| tctttccttc cttccattta tttatttatt tatttattta ttcattcatt | 800 |
| cattcattca ttcattcatt cattcattca ttctttttca cgtgtgtgca | 850 |
| tgatgtgtgt gcatgtctgc atccacatga aagccagagg aggcttattt | 900 |
| gatagagaca tcctgctctg tcacttgcta ccttgctccg ccttggtgag | 950 |
| ggtctttcac tgactctagc tctaggctgt ccagcaatcc agcaatcttc | 1000 |
| ttacgtagtc tagcttttta tatctgtgat ggtgttccca cgcttgccct | 1050 |
| tactcactga gccatccccc tcagctctgt gctttgtgtt tgtttgtttg | 1100 |
| tttgtttgtt tgtttgtttg tttgagacag ggtcttccta tagagtccta | 1150 |
| gctagacttg gaactcacta tgtagatcag gctgtcctca aactttcaat | 1200 |
| gatccgtctg ctgttgcttc tagagtgcta ggaatacgtt tgccataata | 1250 |
| acaagccttc attcctttct gttaaaaatt taaattgtgt ttagactgaa | 1300 |
| ttgtactgct actacagtcc cgtgatttga aacttaaaca cgtaaaagaa | 1350 |
| agacattggg ggaaaataac tcatcttctt acaacttctt ttttgttttt | 1400 |
| gtttttgttt ttcgagacag ggtttctcta tatagccctg gctgtcctgg | 1450 |
| aactcacttt gtagaccagg ctggcctcga actcagaaat cctcctgcct | 1500 |
| ctgcctccca aatgctgggg ttaaaggtgt gcgccaccac gcccggctta | 1550 |
| cttacaactt ctagccaagc aaaaagtttc ctttctcaat ggcaaacaga | 1600 |
| gcaggtatgt acatattaag caacacacat acacactctc tccctcatac | 1650 |
| acacttgttg cttttttccc atcctttaca ggccgactgg tttctcagta | 1700 |
| aagtctctgt ttgtgtcatt acaaaattcc tcgatggctt tggttttctc | 1750 |
| tgtgttccca gcagccatct ggagggaact gcattttcaa gttctccatg | 1800 |
| gtgtactcag cccctccctc tactgatggc tggtcaggtt gtatccagac | 1850 |
| ttttggagct ttatttacaa acgatcatag caatctctcg tgtagcgaga | 1900 |
| tatcaccaag agcacacgag tgaagtgttc cactcaagag ctcaaagagt | 1950 |
| gggagtcggc atcattgtcc agtccatggc acagccctta atttactctg | 2000 |
| cgagatctct gaaatgtctc ctccacagGC TCTGCCTTCC TGTTCCTTGA | 2050 |
| TCCCTCTCTG TCAATCATCA TATCCTACAG TGACATCCCG CCCCACCGAA | 2100 |
| AGCTGCCTGG AGACAAGgtc tgtatcttgc ctaaccttgg gcttctagcg | 2150 |
| catggcatag agaaggggct gagtgcagga gcagagggag tgtctgcttc | 2200 |
| gagctgagta gggtgtatac ttctctccac agACCGATGG AGAGGCCCAC | 2250 |
| CAGCAACTGG AGCGCAGGCA GCTGGGTGCT TGCACTGTGC CTCGCCTGGC | 2300 |
| TGTGGACGTG TCCGGCCTCT GCTTCCTTGC AGCCTCCAAC ATCCGCAGgt | 2350 |
| ctggggaagg tggtgagacg agggaagggg tttttacagg atgcttggag | 2400 |
| agcaagaagg cactccagcc catcccacaa agcaggcaga tctctggggc | 2450 |
| agagtaaaat ctgcagatgt gactcctcca gtcattaccc aggaaactgt | 2500 |
| cctcttcccg tccccagtct agagaaggcc tgggaagacc cattggcacc | 2550 |
| atctcttcca aggggaaggt gttctgcagt gaggccctcg ggcggaggct | 2600 |
| taagtctagt ctcctggcct aattctgggg ttttccttct ggaatggcta | 2650 |
| aagcctctgt aagcggatga tcctccccca cgcattgtaa gctctggggc | 2700 |
| ccttgccctc accctgagaa agtggaaggt gggggctggt gtgggactcc | 2750 |
| agctttcaaa gagtcatgaa cagtgtgtgt tgaaggtctg atcccagcct | 2800 |
| tcacctatat gctagaaaac actgctgctg agccgtagtc ccagcgctgg | 2850 |
| gtttctctta aatgactttt ccatgagcaa ggaggagggg gttgagggcc | 2900 |
| tgcagcaggg taggagagag ggaggagggc agggttggag acctgggtct | 2950 |
| cctgagcttg taattgctgg ctgcatcagT CCTAGTGAAG CAGGGCACCT | 3000 |
| GCGAGGTGAT TGCTGCACAT CGCTGCTGCA ACCGGAACCG CATTGAGGAG | 3050 |
| CGCTCCCAGA CGGTCAAATG CTCCTGCCTG TCCGGCCAGG TGGCTGGCAC | 3100 |
| CACCAGAGCA AAGCCCTCCT GCGTGGACGg tgagtgagag gccaggccct | 3150 |
| gggcagagca gagataagaa agtccactta ggcctcacca tctgctgagg | 3200 |
| gtctagtttc atagctatct taaggctgac cttatctgtc tcaagtggag | 3250 |
| aagagggcag acatggcccg ccctgtgtca tagaggaaga aagctcttgt | 3300 |
| tacagaccat gtacacacct gccacctggc ccattccctc aagccagctc | 3350 |
| ctctggctaa acacttctcg agggaggtcc tcttccacac gtctcttatt | 3400 |
| ctcaacctgc tctgtgagga acatgggatc cctgatacag cacacccact | 3450 |
| ctccttcctt tctgtgcaca ctgtgttctc acgctctggt ggcatccaga | 3500 |
| gcgtcaccat acctgtgctc ccatgccagc ctgtgcccgt ctggatgtgg | 3550 |
| cttggatact agctcatata ccctaaagag ggcactgaag atggtacctc | 3600 |
| tgtgcgaacg acagctgccc ctagcttgat gccctcctcc cgagttggtg | 3650 |
| tgatgcagtg atgaagtttc ctgggataga ccccagtcca cagcctgtgt | 3700 |
| acagtgctgc ctccttggac agcccagagt agggggatcc cagggggaag | 3750 |
| gtggtaatca gcgcctgcag ttggtgggaa ggaagctaca cctcattcac | 3800 |
| agaccagcta tgaccggtgc tgtgctccca gaccttagag ccacacctgc | 3850 |
| ctcctgcccc acagCCTCCA TCGTCCTGCA GAAGTGGTGG TGTCAGATGG | 3900 |
| AGCCCTGCCT GCTGGGAGAG GAGTGTAAGG TGCTCCCGGA CCTGTCAGGG | 3950 |
| TGGAGCTGCA GCAGTGGACA CAAGGTCAAA ACCACCAAGg tattgagacg | 4000 |
| ggtggctcac cctcccctcc tgctcctccc tcacccatga tagcacaggg | 4050 |
| gccaaaccct ggagggccag gccctgctac tggtgatggc atgcctgcct | 4100 |
| tgactttctc ctcttcgatt cccatgggtt tacccctaga aaatatcagg | 4150 |
| ccttttaaaa caaaatggag cagggcacag aggcacacat ctgtaatcct | 4200 |
| agcactcagg aacaagatca ggagttcagt tagcctgggt tgcatgatac | 4250 |
| ctgtatccaa aaatacaaaa acaaacaaac aaaaacaaaa caaaacagga | 4300 |
| aaaaaaaaac cctaaggaaa aaaaatgtag agaactgaga aaattaccta | 4350 |
| gatcaaccag ggatgaattt atccttctat ctgagttcca gaaactgtat | 4400 |
| cctctctgcc tgccacctca ccacagactg tcccctgtca cctacaccac | 4450 |
| tcctacccct tcccctccca cttcctgcct gccctcccct agtcggatgc | 4500 |
| tctgtcctca gtaacagcac ctaagaagat tattatcgtc acagcttgcc | 4550 |
| gcctgcccca caaaacaagg ggtaattaaa attttatcaa actctttagc | 4600 |
| ggttccacaa gtgccgtggc taattagttg tatgtcattg ccaccatcac | 4650 |
| ctgtgcaagt gagtgacatt tccaggcaag ccaaggcagt gtccccgcca | 4700 |
| ccagccccac tacacacagc aagcaggtta actaacagaa gcaaaaccac | 4750 |
| ttctggggtt ctgaggaaaa tggaggtttt cacagaacac acgacactgg | 4800 |
| gaacttcagc agaaaaaggt ttgggggctg gagaggtagc tcagtggtta | 4850 |
| ggagcacttt ttgcttttac agaagaccca ggttcaattt ccagcacctc | 4900 |
| cagtagctta gtactctctg taacacagtc ctaaaggatc caataccctc | 4950 |
| ttctggcctc cttgggcacc acacatgtgg tggacagatg ttcatgcaga | 5000 |
| cacagttctc atatacataa aacttaaaat gtggggaggg agcagttggc | 5050 |
| tgactaaaag gcagggcagc ccatctgtag gcagtaaagg ggggcagcca | 5100 |
| ttctgagaat gtttattaag acttgcaatg tgagacctat cacagagcca | 5150 |
| gggacatttg gcatactgtt acaggcctga aagacagatc actgcctttg | 5200 |
| caatccgtgt agtgactcag aagcaaacgg cagtgaggtt tgcctgacgg | 5250 |
| gtgccagcat ggatggatca ccaggccaca ggtgctcagc ttgggtggtg | 5300 |
| ttaggagact ggcagcggtg acctctcggt gactccccta tgctggggct | 5350 |
| ctctggcatg ccacaatggc cggttccctg ttgctttctc aagccttccc | 5400 |
| ctacctgacg atttgctttc tagGTCACAC GGTAACTCTC GGAGGTCATG | 5450 |
| GCTTAGGTAG GACAGCCTTG ACTGAGCTCG GGACTGAAGA AAGGCCTGGT | 5500 |
| CACCAGACAG CAGATAAGAG GACTTACCTG GACATGTGCC CATGTCAAGT | 5550 |
| GTAACATAGA CCGGCCAGGG CCCTGGGCAG CACTCTGTTC AGCTAAAATG | 5600 |
| CTTGGATCTT TGGCCACACA CTTGAGAGAC CTGTGCTCTC CTATGaacaa | 5650 |
| agtcaacaca caaacctatc cttaaggaat atcttgtcaa gttaaggatg | 5700 |
| ggatgacagg caatttcaac agtttaaagt gtttggtacc gtggc | 5745 |
| Mouse NsG31 (801 bp; CDS = 192-590) | |

(SEQ ID NO 8)
>gi|32967242|gb|AY325122.1|Mus musculus TAFA3 mRNA, complete
cds
TCTCTCTCCATTCTGAACAACCGCCATATTGTTCATCAGACGGAACAAGTCTGAGACCAG
CAGATGAACAGGCAGAGGCCTGAAGACCCCAGGAACAGCTCTGCCTTCCTTGTTCCTTGA
TCCCTCTCTGTCAATCATCATATCCTACAGTGACATCCCGCCCCACCGAAAGCTGCCTGG
AGACAAGACCGATGGAGAGGCCCACCAGCAACTGGAGCGCAGGCAGCTGGGTGCTTGCAC
TGTGCCTCGCCTGGCTGTGGACGTGTCCGGCCTCTGCTTCCTTGCAGCCTCCAACATCCG
CAGTCCTAGTGAAGCAGGGCACCTGCGAGGTGATTGCTGCACATCGCTGCTGCAACCGGA
ACCGCATTGAGGAGCGCTCCCAGACGGTCAAATGCTCCTGCCTGTCCGGCCAGGTGGCTG
GCACCACCAGAGCAAAGCCCTCCTGCGTGGACGCCTCCATCGTCCTGCAGAAGTGGTGGT
GTCAGATGGAGCCCTGCCTGCTGGGAGAGGAGTGTAAGGTGCTCCCGGACCTGTCAGGGT
GGAGCTGCAGCAGTGGACACAAGGTCAAAACCACCAAGGTCACACGGTAACTCTCGGAGG
TCATGGCTTAGGTAGGACAGCCTTGACTGAGCTCGGGACTGAAGAAAGGCCTGGTCACCA
GACAGCAGATAAGAGGACTTACCTGGACATGTGCCCATGTCAAGTGTAACATAGACCGGC
CAGGGCCCCTGGGCAGCACTCTGTTCAGCTAAAATGCTTGGATCTTTGGCCACACACTTG
AGAGACCTGTGCTCTCCTATG
Mouse NsG31 full length amino acid
(SEQ ID NO 9)
>IPI00380405.1 REFSEQ_NP: NP_899047 TREMBL:Q7TPG6 Tax_Id =
10090 Ensembl_locations (Chr-bp): None TAFA3
MERPTSNWSA GSWVLALCLA WLWTCPASAS LQPPTSAVLV KQGTCEVIAA
HRCCNRNRIE ERSQTVKCSC LSGQVAGTTR AKPSCVDASI VLQKWWCQME
PCLLGEECKV LPDLSGWSCS
SGHKVKTTKV TR
Mouse NsG31 mature protein
(SEQ ID NO 10)
SLQPPTSAVL VKQGTCEVIA AHRCCNRNRI EERSQTVKCS CLSGQVAGTT
RAKPSCVDAS IVLQKWWCQM EPCLLGEECK VLPDLSGWSC SSGHKVKTTK
VTR
Mouse NsGS1 Cys1-Cys10 fragment
(SEQ ID NO 11)
CEVIAAHRCC NRNRIEERSQ TVKCSCLSGQ VAGTTRAKPS CVDASIVLQK
WWCQMEPCLL GEECKVLPDL SGWSC
Mouse NsG31 core polypeptide fragment
(SEQ ID NO 12)
GTCEVIAAHR CCNRNRIEER SQTVKCSCLS GQVAGTTRAK PSCVDASIVL
QKWWCQMEPC LLGEECKVLP DLSGWSCSSG HKVKTTK

In human beings an alternative splice variant exists, in which the transcript contains an extra 68 bases in exon 4, resulting in a frameshift mutation compared to NsG31. The corresponding protein is referred to in the following as NsG31-long

### Analysis of Splice Variants of NsG31.

NetGene2 was used to predict potential acceptor splice sites around the variant splice forms of NsG31-long and NsG31 (http://www.cbs.dtu.dk/services/NetGene2/, Prediction of Human mRNA Donor and Acceptor Sites from the DNA Sequence. Brunak, S., Engelbrecht, J., and Knudsen, S., Journal of Molecular Biology, 1991, 220, 49-65.)

The resulting cDNA from NsG31-long adds an additional 68 nt to the transcript and causes the mRNA to be translated in a different reading frame. The variant acceptor sites of the two splice forms received the following prediction scores from Netgene2. Predicted acceptor splice sites for human NsG31-long and human NsG31

| | position | | |
|---|---|---|---|
| Gene | seq id no. 1 | Prob. | Splice site |
| NsG31-long | 3468 | 0.26 | TCATTCGCAG\check{ }ACCTGCTCTT |
| NsG31 | 3536 | 0.90 | GCTCCCACAG\check{ }CCTCCATCGT |

| | |
|---|---|
| tacgtcccca ggcctcaggc tgagtactgc ctctctgggc agcctggagt | 3400 |
| aggggaaaga caagggaaat gcatggctca aggccagggg tgtgtggaaa | 3450 |
| cgtgcacctc attcgcagACCTGCTCTTGG CTGCCCACTG TGCTCGTAGA | 3500 |
| GACCCTAGAG CTGCACTCCG CCTCCTGCTC CCACAGCCTC CATCGTCCTG | 3550 |
| CAGAGATGGT GGTGTCAGAT GGAGCCCTGC CTGCCGGGGG AGGAGTGTAA | 3600 |
| GGTGCTCCCG GACCTGTCGG GATGGAGCTG CAGCAGTGGA CACAAAGTCA | 3650 |
| AAACCACCAA Ggtaccctgg gtgggcacct catcccaccc tccccttccc | 3700 |
| tctctaccaa gggcagaaaa gggggcacag gagccttgga ggtctaggat | 3750 |
| gtcagtgtcc caaggtgaga tcgtgaacca tgaaattcac agacagtcct | 3800 |

(positions, underlined and in bold, correspond to 3468 and 3536 in SEQ ID No. 1 and 13). Thus, the probability for the short version of NsG31 is higher than for the alternative splice variant.

| Human NsG31-long genomic nucleotide sequence; exons in CAPS. | |
|---|---|
| (SEQ ID NO 13) | |
| ccaccccctt gccctctccc aggttctcct tcaccccctg cagagccatc | 50 |
| ggctccctct atcctttgca accccctatg aatctttcct acttgtccag | 100 |
| GTGGAGTGAG TCTGAGGACA GCAGATGAAC AGACAGAAAC TGAAAGATCC | 150 |
| CCAAAAAGgt aagtggggag acattgaaga tggggagtag cgggggtgga | 200 |
| cagagatcaa gtaccctatc tgaggagcaa ctgggtccta gcagttggtt | 250 |
| tggcctctgg tattcccaaa gctgcactgg cacttgtggg ggcccagcag | 300 |
| tggtccaggc tcacccctga gcttttggcc ttgctgcatc ctgggacttc | 350 |
| tcctgctccc ttcaatggct gggcagctcc tagctactct gtgcctcttg | 400 |
| cttccttagg attccctcca tttccagcct acccttcagg gtagctttgg | 450 |
| gaggcccaaa ccatcaacga tgaaagagga aaaggaaagg ggaagagaac | 500 |
| agaggcaagg tggtgcagtg ggcagagggc aggcttctag gctctgaact | 550 |
| cttccaccca catgctgcct ggccttgggc aagtcatttg acttctgtta | 600 |
| gcctcggttt cacctcttcc tttttaaatt taaattctgt ttaaattgaa | 650 |
| gtgctactgc attcccgtgg ttggcagctc aaaaagcaag aaataaatca | 700 |
| ggagaaaaag tctctcttct acctccatct cccaaccaac cagtttccct | 750 |
| tttggatggc aaacaatatt actagtttta aaatatattt ttcaaagcta | 800 |
| gtttatgcat atctgagcaa catagataat ccttccttta tacacatttt | 850 |
| attttattta tttttgagct agggtctcac tctgttgccc aggctggagt | 900 |
| gcagtagcac agtcatagat cactgcagcc tcaaactcct gatcctctca | 950 |
| cctcagcctc tggagtagct gggactatag gtgtgcacca ccatgcctga | 1000 |
| ctaatttttt aattttttgt agagatgggg tcttgctatg ttgcctaggc | 1050 |
| tggtctcgaa cttctggcct caagtgatcc tcctgccttg gcctcccaaa | 1100 |
| gtgctggaat tagaggcatg aaccaccact cctggcctgc cttatacaca | 1150 |
| tcgtatacac accattctac actttgcttt ttttccccct taacagtgga | 1200 |
| ttatcactgg agatcactgc atgtgtcagg acataaagca attccccatt | 1250 |
| ggttttttta cagctatctg gatgggactg catttgtaca gactctttac | 1300 |
| gatgtattca gccagtcttt tactgatggt tgtttaggtt atgtcctagc | 1350 |
| tttttgagct ttggttttct catctgtaaa acaggtaata ataatgttgc | 1400 |
| tttgtaggac agttgtgaag aattcaagag tgtgcatgtg aagtgctgga | 1450 |
| accaagcact caaagagtag gagtcagtat tattgtccct cccatgtcac | 1500 |
| gaccctccat gggcccatag ctagttcatc ctccaagatg cctcttaaat | 1550 |
| gttgtctcct ccaaaggctc tccttttctt cctgcagcac tcactactgc | 1600 |
| ctgcatgact ctgtgccagg tcgaaagatg cctggggaca aggtgtgcct | 1650 |
| cctccccatc tttgtgcctc ccagagtgtg tggcacagag aaggtgccca | 1700 |
| ggtgcacagg ggagcttctg cctggagctg agcagaatgt gtgcttctct | 1750 |
| ccgcagGATG AGTGAGAGGG TCGAGCGGAA CTGGAGCACG GGCGGCTGGC | 1800 |
| TGCTGGCACT GTGCCTGGCC TGGCTGTGGA CCCACCTGAC CTTGGCTGCC | 1850 |
| TTGCAGCCTC CCACTGCCAC AGgtttggag gaggtggcag ggcccgggga | 1900 |
| gggagtttcc caggacacct ggggagctgg acaccactcc accgtgttcc | 1950 |
| atagcaggca ggtctcccag gcagagtaaa atccagagat tctaggggtt | 2000 |
| ccggctcctc ctctgctctt tacccaggaa tctctgccca ccccagctgt | 2050 |
| cccgccccag agaaggtctg ggcagaccct ttgctcacct ctccttccag | 2100 |
| gctggaaggt gttctgtagt gagagaggcc ctcgggtgag ggcttatctc | 2150 |
| tagcctcctg gcctaattct atggttttcc ttctgggatg gctaaagcct | 2200 |
| gtgtaagcgg atgatcctcc ccacgcattg taagccctgg ggcccttgcc | 2250 |
| ctcaccctga ggaagctggg gaggcggggc tggtgcggga ctctgtcttc | 2300 |
| tgcccagggg ccgtgctctg ccaaagggtg aagagcacag gcctcacagg | 2350 |
| ggagggagga agaataggag ctctgtgaag cggcgggaag atttcatcca | 2400 |
| cttcctcctc ccctgccact ggcagtctct tccatagccc tccctgcagc | 2450 |
| agggaggagg ggctggaggc cagcccgggt gggcgggagg gagcggggtc | 2500 |
| gggcgtgttg ggcgtctgat cctgggcggc tcccctcagT GCTTGTGCAG | 2550 |
| CAGGGCACCT GCGAGGTGAT TGCGGCTCAC CGCTGCTGCA ACCGGAACCG | 2600 |
| CATCGAGGAG CGCTCCCAGA CGGTGAAATG CTCCTGTTTT TCTGGCCAGG | 2650 |
| TGGCCGGCAC CACGCGGGCA AAGCCCTCCT GCGTGGACGg tgagtgagag | 2700 |
| gccaggaccc gggcagggcc ctgagcagag ccataaggag gcctgttcag | 2750 |
| gcctcgccat ctgctgggga tacaatttca tacccgtctc agggctttca | 2800 |
| tgagcagtct caaatggtgg ggccccctct gggagaagag ggcagacgtg | 2850 |
| gccatccctg tgtcatagat gagaaaagct cttgttatgc gcacagcctc | 2900 |
| ccacactcac gtgccctctg cctgttgcac acacaggagc gttccctcac | 2950 |
| acagccccac tcctctcccc acacacttcc tccagctcct tcccctgctg | 3000 |
| ctccttcccc tggctgtctc agcctactct cctctccttc tcagcccttc | 3050 |
| tctcacactt tcctatgtgg acacgtgtga tccttcacac ccacactcat | 3100 |
| taccctaaca ctcatttggc accccataca catgcccact gtgtcacgcg | 3150 |
| cataccagct atgccagcta tgcactcctg gtctgtagac ccctccagac | 3200 |
| gactcacacc cttgctcaca caccacagag ggagtatact ggttgtgcat | 3250 |
| cgatgctagt gacatggtca ccttagagac atttttgcct gccttcaccc | 3300 |
| gtcactgtct ggggagatgc agggatcggt gggattaaag ttgagagggt | 3350 |
| tacgtcccca ggcctcaggc tgagtactgc ctctctgggc agcctggagt | 3400 |
| aggggaaaga caagggaaat gcatggctca aggccagggg tgtgtggaaa | 3450 |
| cgtgcacctc attcgcagAC CTGCTCTTGG CTGCCCACTG TGCTCGTAGA | 3500 |
| GACCCTAGAG CTGCACTCCG CCTCCTGCTC CCACAGCCTC CATCGTCCTG | 3550 |
| CAGAGATGGT GGTGTCAGAT GGAGCCCTGC CTGCCGGGGG AGGAGTGTAA | 3600 |
| GGTGCTCCCG GACCTGTCGG GATGGAGCTG CAGCAGTGGA CACAAAGTCA | 3650 |
| AAACCACCAA Ggtaccctgg gtgggcacct catcccaccc tccccttccc | 3700 |
| tctctaccaa gggcagaaaa gggggcacag gagccttgga ggtctaggat | 3750 |
| gtcagtgtcc caaggtgaga tcgtgaacca tgaaattcac agacagtcct | 3800 |
| ctcaggtctg ttctcactgg ggagtggagt taggggggtc aggacttgaa | 3850 |
| atccagtgtg tcctgggaag attcttgatg tccatatacg ctggtttcct | 3900 |
| cctgtctgcc tggaattagt ttgcctccag aaaatatcag gcctataaaa | 3950 |
| acatagtcca tggaatacta tgcagccata aaaaatgatg agttcatgtc | 4000 |
| ctttgtaggg acatggatga aattggaaat catcattctc agtaaactat | 4050 |
| cgcaagaaca aaaaaccaaa caccgcatat tctcactcat aggtgggaac | 4100 |
| tgaacaatga gatcacatgg acacaggaag gggaatatca cactctgggg | 4150 |
| actgtggtgg ggtgggggga ggggggaggg gtagcattgg gagatatacc | 4200 |
| taatgctaga tgacgagtta gtgggtgcag cgcaccagca tggcacatgt | 4250 |
| atacatatgt aactaacgtg cacaatgtgc acatgtaccc taaaacgtaa | 4300 |
| agtataattt aaaaaaaaaa aaacaacata ttagagcagc aaaaaaaaaa | 4350 |
| aaaaaaaaaa aaaagagctt aaaacagaac taccattcaa cccagccatc | 4400 |
| ctattactgg tatataccca aaggaaaata aatcattttg ccaaaaagac | 4450 |
| acatgcactt gtatgttcat caaagcagta ttcataatag caagggcata | 4500 |
| taatcagtct aggtgccaat caacattgaa taggataaag aaaatgtggc | 4550 |
| acttatatac catagaatac tacacagcca taaaaaaaat aatgttttct | 4600 |
| gtaacaccct ggttgcagct gaaggccatg atcctaaggg acttagcatg | 4650 |
| gcaacggaaa atcaaatacc acatgttctc acttctgaga gctaaacatt | 4700 |
| gggtacacat ggacataaag atgggaacag tggacgatgg tgactaccag | 4750 |
| agcagtgaga gagggagggg agcaagggct gaaaaactac ctattgggtc | 4800 |
| ctatgcttac tacatgggta atgagatcaa tggcactcca aacctcagtg | 4850 |
| tcatgcaata tactcatgta acaaacctgc acatgtaatc cctgaatcta | 4900 |
| aaataaaagt tgaaatatta aaaaaaaaaa aaaaaaaaac atagtcgaga | 4950 |
| atgggaaaaa ttataagata attgagggag gaattgctac ttctacctga | 5000 |
| gctccaggta cagtagtcac tctgtcactc acctctgccg tgaccatccc | 5050 |
| ccacccccac cccattccca ctgttgttga ctccctacag ttcataccta | 5100 |
| ccttcctcta gtcagatttc tctgtccact gtcacagcac ccaagaggat | 5150 |
| tattattttc atggtttgct gcctgctcca cagaacaagg ggtaattaaa | 5200 |
| ttttttatca aactcgttag cagctccatg tgtgtcatgg ctaatcagcc | 5250 |
| gcattgctct gccgtcacca ttggtacaga agagtgacac tgcagggcaa | 5300 |
| gccagggttc tgcctttggc tttccttccc tcccaccaca cacagcaaac | 5350 |
| aagcaaacac ataagaaaat agagaaggag ggctgggtgc ggtggctcat | 5400 |
| gcctgtaatc ccagcacttt gggagacaga ggtgggtgaa tcaagaggtc | 5450 |
| aggagttcaa gaccaacctg gtcaacatgg tgaaaacccg tctctactaa | 5500 |
| aaatacaaaa attagctggg cataatggca cacgcctgta atcccagcta | 5550 |
| cttgggaggc tgaggcagga gaattgcttg aacctaggag gcagaagttg | 5600 |
| cggtaagcag agatcatgcc actgcactcc agtctgggcg acagagtgag | 5650 |
| actccatctc aaaaaaagaa aaaaagaaaa aaaaatagag aaggaaaggc | 5700 |
| agcctttggc ttctgaagag agcaaccatt ttcacagagc acacaacaaa | 5750 |
| aggagctaca gcaagaaaaa gtttggctct aaagtagagc atcctctctc | 5800 |
| tatccggagg cagggatgag ggacagccat tctgagtata tttaagatca | 5850 |
| gaagagaatg agacccacta cagagcttgt gacatttgtc accatgttat | 5900 |
| aggtttgggt ttgtggcctg taaggaagtg cttgcaattt gggtagctac | 5950 |
| tctgaagcaa acagtgggga ggcttgtctg acgtatgttt gcatgggtca | 6000 |
| ccaggccatg ctttcccatt ccggcctgct ttagggtgag aggattggca | 6050 |
| gcaatgtcct ctaagtgacc tgcatattct gggtagtctc tggcatgctg | 6100 |
| gaatggaata ggcattccct tctctaacct taccctctcg cttcttcacc | 6150 |
| agGTCACACG ATAGCTCTTG GGGGTCACGG CCTGGACAAG AAAGGCTTGA | 6200 |
| CTGAGCCGTG AACTGAAGAA TGGTATCCAG TCATCAGCCA GGAAAGATGG | 6250 |
| GGATTCACTT ACATGCCTCA TGTCAAATGC AGCATCAGTC TTTCCGGGGC | 6300 |
| ATTTCAGTTA AGCTGCTCAG CAGATATGGA TGGATCTGCA ATCACATACC | 6350 |
| TAATGTGGAG CTGGGCTTTT CTGGAGACAC GAAGGTCAAC ACACAATTCC | 6400 |
| TGCCCTTAAG GAATGTCCAG TTGAATTGGA GAGTTGATGA CAGACAATTT | 6450 |
| AGATAATTTA GGTTAAAGTA CTTGATACCA GACTGCGGCT TCTGGGCCAC | 6500 |
| ATGCTATGGC ATGATGGGGG TTTGGGAATG AGATTCCCAC AGTTCTTCAG | 6550 |
| ATACCCTGTG GCCACAGGGC ATAGAAACAA GAGGTCACAT TCAGCACCCA | 6600 |
| CCACCTCCCT CTTTCGCATC AGTtCTGAAT CCCCAGCAAG CTGTTAACAT | 6650 |
| GTTGCAGGAA AACACTCTCC CCTTATGCCA GACCAGCAGT ATCTCATTTG | 6700 |
| GATGGGATTG GATTGACTTG CGGAAGGAAA GTAAAAATAA AGCCAAATAA | 6750 |
| CTTCCCAAAt ctttggcaca ggtatgggcc attcattggg tatgggatgg | 6800 |
| acaggacttt aaaatcatct ccatccaagc acctcaggtt tttgtttgtt | 6850 |
| tgcttattt | 6859 |

NB I There is an inconsistency at position 163 ‘C’ in the human NsG31-long cDNA, which in the genomic sequence is a ‘T’. This is in the coding region and therefore two different codons can appear, but the protein sequence will not be affected.

163 C = > CTG -> Leu
163 T = > TTG -> Leu
Human NsG31-long cDNA (1144 bp; CDS = 70-579)
(SEQ ID NO 14)
>gi|32967236|gb|AY325119.1|Homo sapiens TAFA3.2 mRNA, complete cds,
alternatively spliced
GGTGGAATTCGTGGAGTGAGTCTGAGGACAGCAGATGAACAGACAGAAACTGAAAGATCCCCAAAAAGGA
TGAGTGAGAGGGTCGAGCGGAACTGGAGCACGGGCGGCTGGCTGCTGGCACTGTGCCTGGCCTGGCTGTG
GACCCACCTGACCTTGGCTGCCCTGCAGCCTCCCACTGCCACAGTGCTTGTGCAGCAGGGCACCTGCGAG
GTGATTGCGGCTCACCGCTGCTGCAACCGGAACCGCATCGAGGAGCGCTCCCAGACGGTGAAATGCTCCT
GTTTTTCTGGCCAGGTGGCCGGCACCACGCGGGCAAAGCCCTCCTGCGTGGACGACCTGCTCTTGGCTGC
CCACTGTGCTCGTAGAGACCCTAGAGCTGCACTCCGCCTCCTGCTCCCACAGCCTCCATCGTCCTGCAGA
GATGGTGGTGTCAGATGGAGCCCTGCCTGCCGGGGGAGGAGTGTAAGGTGCTCCCGGACCTGTCGGGATG
GAGCTGCAGCAGTGGACACAAAGTCAAAACCACCAAGGTCACACGATAGCTCTTGGGGGTCACGGCCTGG
ACAAGAAAGGCTTGACTGAGCCGTGAACTGAAGAATGGTATCCAGTCATCAGCCAGGAAAGATGGGGATT
CACTTACATGCCTCATGTCAAATGCAGCATCAGTCTTTCCGGGGCATTTCAGTTAAGCTGCTCAGCAGAT
ATGGATGGATCTGCAATCACATACCTAATGTGGAGCTGGGCTTTTCTGGAGACACGAAGGTCAACACACA
ATTCCTGCCCTTAAGGAATGTCCAGTTGAATTGGAGAGTTGATGACAGACAATTTAGATAATTTAGGTTA
AAGTACTTGATACCAGACTGCGGCTTCTGGGCCACATGCTATGGCATGATGGGGGTTTGGGAATGAGATT
CCCACAGTTCTTCAGATACCCTGTGGCCACAGGGCATAGAAACAAGAGGTCACATTCAGCACCCACCACC
TCCCTCTTTCGCATCAGTTCTGAATCCCCAGCAAGCTGTTAACATGTTGCAGGAAAACACTCTCCCCTTA
TGCCAGACCAGCAGTATCTCATTTGGATGGGATTGGATTGACTTGCGGAAGGAAAGTAAAAATAAAGCCA
AATAACTTCCCAAAAAAAAAAAAA
Human NsG31-long full length amino acid sequence
(SEQ ID NO 15)
>emb|AY325119|AY325119 Homo sapiens TAFA3.2 mRNA, complete cds,
alternatively spliced.
MSERVERNWS TGGWLLALCL AWLWTHLTLA ALQPPTATVL VQQGTCEVIA AHRCCNRNRI
EERSQTVKCS CFSGQVAGTT RAKPSCVDDL LLAAHCARRD PRAALRLLLP QPPSSCRDGG
VRWSPACRGR SVRCSRTCRD GAAAVDTKSK PPRSHDSSWG SRPGQERLD
Human NsG31-long mature protein
(SEQ ID NO 16)
ALQPPTATVL VQQGTCEVIA AHRCCNRNRI EERSQTVKCS CFSGQVAGTT RAKPSCVDDL
LLAAHCARRD PRAALRLLLP QPPSSCRDGG VRWSPACRGR SVRCSRTCRD GAAAVDTKSK
PPRSHDSSWG SRPGQERLD

### Example 1b

### NsG29 Sequences

SEQ ID NO 17, human NsG29 cDNA
SEQ ID NO 18, human NsG29 full length amino acid sequence
SEQ ID No. 19, human NsG29, mature protein
SEQ ID No. 20, human NsG29, Cys1-Cys10 peptide fragment
SEQ ID No. 21 human NsG29, core polypeptide fragment
SEQ ID NO 22, mouse NsG29 cDNA
SEQ ID NO 23, mouse NsG29 full length amino acid sequence

Human NsG29 (815 bp; CDS = 217-618)
(SEQ ID NO 17)
>gi|32967226|gb|AY325114.1|Homo sapiens TAFA1 mRNA, complete cds
GACATCAGAACCCCAGGCTCTCCAGCCTTTGGACTTCAGGACTGACACAAGCAACCTGCTGGGTTCTTAG
GCCTTTGGCTTGGTACTGAGACTTACACCATCAGCTTCCCTGGTCCTGAGACTTTTGGACTTGGATTGAG
CCACGCTACTGGCATCCCAGGATCTCCAGCTTGCAGACAGCCTGTCGTGGGACTTCACAGCCTCCATAAT
TATAGAATGGCAATGGTCTCTGCGATGTCCTGGGTCCTGTATTTGTGGATAAGTGCTTGTGCAATGCTAC
TCTGCCATGGATCCCTTCAGCACACTTTCCAGCAGCATCACCTGCACAGACCAGAAGGAGGGACGTGTGA
AGTGATAGCAGCACACCGATGTTGTAACAAGAATCGCATTGAGGAGCGGTCACAAACAGTAAAGTGTTCC
TGTCTACCTGGAAAAGTGGCTGGAACAACAAGAAACCGGCCTTCTTGCGTCGATGCCTCCATAGTGATTG
GGAAATGGTGGTGTGAGATGGAGCCTTGCCTAGAAGGAGAAGAATGTAAGACACTCCCTGACAATTCTGG
ATGGATGTGCGCAACAGGCAACAAAATTAAGACCACGAGAATTCACCCAAGAACCTAACAGAAGCATTTG
TGGTAGTAAAGGAAAACCAACCCTCTGGAAAATACATTTTGAGAATCTCAAACATCTCACATATATACAA
GCCAAATGGATTTCTTACTTGCACTTTGACTGGCTACCAGATAATCACAGTGCGTTTACTGTGTGTAACG
AAATATCCTACAGTGAGAAGACACAGCGTTTTGGCAACACCATGG
Human NsG29 full length amino acid sequence
(SEQ ID NO 18)
>IPI00376089.1 TREMBL: Q7Z5A9 ENSEMBL: ENSP00000328220 Tax_Id = 9606
TAFA1
MAMVSAMSWV LYLWISACAM LLCHGSLQHT FQQHHLHRPE GGTCEVIAAH RCCNKNRIEE
RSQTVKCSCL PGKVAGTTRN RPSCVDASIV IGKWWCEMEP CLEGEECKTL PDNSGWMCAT
GNKIKTTRIH PRT

A possible SNP has been found at position 92, resulting in W instead of G.

Human NsG29, mature protein
(SEQ ID No 19)
SLQHTFQQHH LHRPEGGTCE VIAAHRCCNK NRIEERSQTV KCSCLPGKVA GTTRNRPSCV
DASIVIGKWW CEMEPCLEGE ECKTLPDNSG WMCATGNKIK TTRIHPRT
Human NsG29, Cys1-Cys10 fragment
(SEQ ID No. 20)
CEVIAAHRCC NKNRIEERSQ TVKCSCLPGK VAGTTRNRPS CVDASIVIGK WWCEMEPCLE
GEECKTLPDN SGWMC
Human NsG29, core polypeptide fragment
(SEQ ID No 21)
GTCEVIAAHR CCNKNRIEER SQTVKCSCLP GKVAGTTRNR PSCVDASIVI GKWWCEMEPC
LEGEECKTLP DNSGWMCATG NKIKTTR
Mouse NsG29 (801 bp; CDS = 238-639)
(SEQ ID NO 22)
>gi|32967238|gb|AY325120.1|Mus musculus TAFA1 mRNA, complete cds
TACAGTGAACGAGCCCAACGTCTTCACAACTGTGGAAACGGGACTGGAAGAGAAAAGCTTGCCTTTTTTT
CTTTTCTTTTCTTTTCTTTTTTTTTTTTCAATTCTTAATAAAGAATTGCTGGGAAGCATTCTCTTTGAAA
AATCTCAGAACTGTGGCACAGATGGATTTTAAAAAGTGTTAGCTCTTTCCAATGAGCACTAGGAGGGTTC
CCTGCTCTTGGCTGGATTTTTCAGAGAATGGCAATGGTCTCTGCAATGTCCTGGGCCCTGTACTTGTGGA
TAAGTGCTTGTGCGATGCTGCTCTGCCATGGGTCACTCCAACACACCTTCCAGCAGCATCACCTGCACCG
GCCAGAAGGAGGGACCTGTGAAGTGATCGCGGCCCACAGGTGTTGTAACAAGAACCGCATCGAGGAGCGG
TCACAAACAGTGAAGTGTTCCTGTTTACCTGGGAAAGTGGCTGGGACAACAAGAAACCGACCTTCCTGTG
TGGATGCCTCCATAGTAATTGGGAAATGGTGGTGTGAGATGGAGCCCTGCCTAGAAGGAGAAGAATGTAA
GACACTCCCTGACAATTCTGGATGGATGTGTGCTACAGGCAACAAGATTAAGACTACACGAATTCACCCA
AGAACCTAACAGAAGCATTTGTTATATAAATAGGAAAAAGAACAACCTGTGGAATATACGTTGTGAGGAT
TTAAAACATCTTCCATAGTTGCAAGCCAAGTGGATCTCTTATCTGCACTTTGGTTACCAGATAACCACAG
TGCACTTACTCTGATACACAGTATCCCAAAA
Mouse NsG29 full length amino acid
(SEQ ID NO 23)
>IPI00380407.1 REFSEQ_NP: NP_877960 TREMBL: Q7TPG8 Tax_Id = 10090
Ensembl_locations (Chr-bp): None TAFA1
MAMVSAMSWA LYLWISACAM LLCHGSLQHT FQQHHLHRPE GGTCEVIAAH RCCNKNRIEE
RSQTVKCSCL PGKVAGTTRN RPSCVDASIV IGKWWCEMEP CLEGEECKTL PDNSGWMCAT
CNKIKTTRIH PRT

### Example 2a

### Bioinformatics, NsG31

NsG31 is a 133 amino acid secreted growth factor protein or hormone. The mouse (IPI00380405.1 version 1.22) homologue has a full length of 132 amino acids with a % sequence identity of 85.7.

Protein processing: Human NsG31 contains a N-terminal signal peptide sequence of 30 amino acids which is cleaved at the sequence motif TLA-AL. This signal peptide cleavage site is predicted by the SignalP method (Nielsen et al., 1997) and the output graph shown in FIG. 2. A signal peptide cleavage site is found at a similar location in the mouse NsG31 after position 29. The same signal sequence is present in the human alternative splice form NsG31-long.

The calculations of sequence identity below (Tables 3a and 4a) have been made with the align0 program, using a BLOSUM50 matrix and gap penalties −12/−2.

Table 3a shows the % sequence identity between full length human NsG31 versus mouse.

| Sequence | % id |
|---|---|
| human | — |
| mouse | 85.7 |

Table 4a shows the % sequence identity between human and mouse NsG31 sequences after removal of N-terminal signal peptide.

| Sequence | % id |
|---|---|
| human | — |
| mouse | 93.2 |

### Protein Function:

NsG31 belongs to the category of proteins acting as hormones or growth factors. This notion is supported by predictions by the ProtFun protein function prediction server (Jensen et al., 2002 & 2003), which provides odds scores above 1 for the hormone category as shown in FIG. 3. The mouse homologue provides odds scores above 1 for both the hormone and the growth factor category. Human NsG31-long also provides odds score above 1 for the hormone category.

### Example 2b

### Bioinformatics, NsG29

Human NsG29 is a 133 amino acid secreted growth factor protein or hormone and it is very similar the mouse NsG29 (IPI00380407.1 version 1.20) with a length of 133 and sequence identity of 99.2%.

The sequence identities below (Table 3b and 4b) have been calculated with the align0 program, using a BLOSUM50 matrix and gap penalties −12/-2.

Table 3b shows the % sequence identity between full length human NsG29 (SEQ ID NO 18) versus mouse sequence (SEQ ID NO 23).

| Sequence | % id |
|---|---|
| Human | — |
| Mouse | 99.2 |

Table 4b shows the % sequence identity between human and mouse NsG29 sequences after removal of N-terminal signal peptide.

| Sequence | % id |
|---|---|
| Human | — |
| Mouse | 100.0 |

Human NsG29 contains a N-terminal signal peptide sequence of 25 amino acids which is cleaved at the sequence motif CHG-SL. This signal peptide cleavage site is predicted by the SignalP method (Nielsen et al., 1997) and the output graph shown in FIG. 16. The mouse NsG29 cleavage site is also found after position 25.

Human NsG29 belongs to the category of proteins acting as hormones or growth factors. This notion is supported by predictions by the ProtFun protein function prediction server (Jensen et al., 2002 & 2003), which provides scores above 1 for both these categories for both mouse and human NsG29 as shown in FIG. 17. Also the variant human NsG29 with a G→W mutation at position 92 provides scores above 1 for both the hormone and growth factor ontology classes.

In general, an odds score of 1 indicates that the score is similar to “background” score, and no preference for a particular class is therefore predicted. Odds above 1 indicate that there is a significant prediction indicating that the protein indeed does belong to the predicted gene ontology class. The higher the odds score, the more certain the prediction.

The ProtFun method predicts protein function based on sequence-derived features as opposed to sequence similarity. Features which are important for discriminating between the ‘growth factor/hormone’ classes versus all other classes are: protein sorting potential, protein targeting potential, signal peptide potential, low complexity regions, secondary protein structure, number of negative residues and number of atoms (Jensen et al., 2003).

### REFERENCES

ProP: Prediction of proprotein convertase cleavage sites. Peter Duckert, Søren Brunak and Nikolaj Blom. Protein Engineering, Design and Selection: 17: 107-112, 2004
SignalP: Identification of prokaryotic and eukaryotic signal peptides and prediction of their cleavage sites. Henrik Nielsen, Jacob Engelbrecht, Søren Brunak and Gunnar von Heijne,
Protein Engineering 10, 1-6 (1997).
ProtFun: Ab initio prediction of human orphan protein function from post-translational modifications and localization features. L. Juhl Jensen, R. Gupta, N. Blom, D. Devos, J. Tamames, C. Kesmir, H. Nielsen, H. H. Steærfeldt, K. Rapacki, C. Workman, C. A. F. Andersen, S. Knudsen, A. Krogh, A. Valencia and S. Brunak. J. Mol. Biol., 319:1257-1265, 2002.
Prediction of human protein function according to Gene Ontology categories, L. J. Jensen, R. Gupta, H. H. Stærfeldt, S. Brunak, Bloinformatics, 19, 635-642 (2003).
align0 Optimal alignments in linear space. Myers, E. W. and Miller, W. Comput. Appl. Biosci., 4, 11-17 (1998).

### Example 3a

### Cloning of Both Splice Variants of Human NsG31, hNsG31-Long and hNsG31

Both splice variants of hNsG31 were PCR amplified in one go using human retina cDNA as template and the following primers:

5′ primer:
5′-CGGGATCCGCCACCATGAGTGAGAGGGTCGAGCGG-3′
3′ primer:
5′-TATACTCGAGGAAATGCCCCGGAAAGACTG-3′.

The first oligo included a Kozak consensus sequence (Marylin Kozak, Nucleic Acids Res. 1987 Oct. 26; 15(20): 8125-48) in addition to a BamHI restriction site. The second oligo included a XhoI restriction site. Four identical PCR reactions were set up using 4×80 ng human retina cDNA as template in a 50 μl reaction volume. A proofreading polymerase (pfu-turbo polymerase, Stratagene) was applied for the PCR amplification, with the following amplification profile: pre-denaturation step: 96° C., 1′ followed by 35 3-step cycles: denaturation step: 96° C., 30″; annealing step: 65° C., 30″; elongation step: 72° C., 90″. Then an elongation step: 72° C., 2′ followed by cooling to 4° C.

PCR reactions were pooled and the 611 bp PCR fragment (hNsG31-long) and 543 bp PCR fragment (hNsG31) were purified on a PCR purification column (Roche) and digested with BamHI and XhoI. The now 599 bp and 531 bp BamHI/XhoI-restricted hNsG31-long and BamHI/XhoI hNsG31 PCR fragments were gel-purified from the same gel. Five μg of a lentiviral transfer vector, pHsCXW, (GenBank accession #: AY468-486) is digested with BamHI and XhoI and the vector backbone was gel purified. The BamHI/XhoI hNsG31-long and BamHI/XhoI hNsG31 PCR fragments are ligated into the BamHI and XhoI sites of the pHsCXW lentiviral transfer vector followed by transformation into XL1-B electrocompetent cells.

### Example 3b

### Obtaining a Full Length Coding Sequence for NsG29

NsG29 cDNA was PCR amplified from an IMAGE clone (The I.M.A.G.E. Consortium: “An integrated molecular analysis of genomes and their expression”, Lennon, Auffray, Polymeropoulos, and Soares, [1996], Genomics 33: 151-152) obtained from RZPD, Berlin, Germany (RZPD clone ID: IRAKp961B0349Q) using the following primers:

| | |
|---|---|
| 5′ primer: | 5′-GCTCTAGAGAGAATGGCAATGGTCTCTG-3′ |
| 3′ primer: | 5′-TATACTCGAGGCTGTGTCTTCTCACTGTAG-3′ |

Three identical PCR reactions were set up with 50 ng/μl of the RZPD clone as DNA template in a 50 μl reaction volume. A proofreading polymerase (pfu-turbo polymerase, Stratagene) was applied for the PCR amplification, with the following amplification profile: pre-denaturation step: 95° C., 1′ followed by 35 3-step cycles: denaturation step: 95° C., 30″; annealing step: 55° C., 30″; elongation step: 72° C., 90″. Then an elongation step: 72° C., 2′ followed by cooling to 4° C.

PCR reactions were pooled and the 603 bp NsG29 PCR fragment was agarose gel-purified and cut with XbaI and XhoI. The now 591 bp XbaI/XhoI-restricted NsG29 PCR fragment was gel-purified. Five μg of a lentiviral transfer vector, pHsCXW, (GenBank accession #: AY468-486) was digested with XbaI and XhoI and the vector backbone was gel purified.

The XbaI/XhoI NsG29 PCR fragment was ligated into the XbaI and XhoI sites of the pHsCXW lentiviral transfer vector followed by transformation into XL1-B electrocompetent cells.

### Example 4a

### NsG31 Quantitative Expression Data

### Method

Primers amplifying cDNA fragments of 100-350 bp were designed using CloneManager software. Total RNAs derived from foetal and adult human tissues were purchased from Clontech, Dnase treated to remove residual chromosomal DNA and used as templates for cDNA synthesis using an RnaseH deficient reverse transcriptase. cDNA equivalent to 21 ng total RNA was used for each PCR reaction which were carried out using a DNA engine 2 Opticon light cycler from MJ research.

Real-time PCR was performed in an Opticon-2 thermocycler (MJ Research), using LightCycler-FastStart DNA Master SYBR Green I kit (Roche). Studies were carried out in duplicates using primers 5′ oligo: 5′-AGACCCTAGAGCTGCACTCC-3′, annealing at bp 366 in GenBank sequence: AY325119 and 3′ oligo: 5′-TTCTTGTCCAGGCCGTGACC-3′, annealing at bp 567 in GenBank sequence: AY325119 amplifying a 202 bp fragment of NsG31 spanning one intron between exons 4 and 5 of NsG31 in GenBank sequence: AY325119. This primer pair (NsG1129/1130) captures only the NsG31-long transcript. PCR cycling profile for this reaction consisted of 98° C., 10′ [symbol] 35 cycles: 98° C., 10″ [symbol] 68° C., 20′ [symbol] 72° C., 20″ [symbol] plate read 82° C., 2′.

Another reaction was carried out with another primer-pair (NsG1131/1132) to amplify both the long and the short forms of NsG31 with primers amplifying a 194 bp fragment in the fifth exon of both versions of NsG31. The primers used for this reaction were 5′ oligo: 5′-CACATGCTATGGCATGATGG-3′, annealing at bp 873 in GenBank sequence: AY325119 and 3′ oligo: 5′-TACTGCTGGTCTGGCAAG-3′, annealing at bp 1066 in GenBank sequence: AY325119. PCR cycling profile for this reaction consisted of 98° C., 10′ [symbol] 35 cycles: 98° C., 10′ [symbol] 63° C., 20′ [symbol] 72° C., 20″ [symbol] plate read 78° C., 2″

For Real-Time PCR, a standard curve was prepared by serial dilution of a gel-purified PCR product, prepared using the above primers. The standard curve was used to verify that crossing-point values (C(T)) of all samples were within the exponential range of the PCR reaction and to calculate final expression levels. All. RT-PCR amplifications were performed in a total volume of 10 μl containing 3 mM MgCl₂, 12% sucrose and 1× reaction buffer included in the LightCycler kit. The specificity of the amplification reaction was determined by performing a melting curve analysis of the PCR fragments by slowly raising the temperature from 55° C. to 95° C. with continuous data acquisition.

Expression levels were calculated from C(T) values and standard curves generated from serial dilutions of template DNA (plasmid or PCR product).

For normalization purposes, all cDNAs were subjected to real-time PCR using primers for β₂-microglobulin (B2M, 5′-TGTGCTCGCGCTACTCTCTC-3′ and 5′-CTGAATGCTCCACTTTTTCAATTCT-3′). Standard curves for β₂-microglobulin were prepared similar to NsG31. N-microglobulin gene real-time PCR was done using the same kit as for the target gene, except a different annealing temperature was used.

β₂-microglobulin expression levels were determined from the respective standard curves and the relative expression levels were used to normalize expression levels of the target genes in the tissues that were analyzed. Following normalization, relative expression levels of the target gene were calculated using the tissue with the lowest expression as a reference. Normalized data in FIG. 6 should be interpreted with caution as β₂-microglobulin levels vary between some tissues.

### Analysis of Total RNA Samples (FIGS. 5-6)

### High and Intermediate Expression (C(T) Values<26)

Retina, Dorsal Root Ganglion, Cerebellum, Substantia Nigra, testis

### Low Expression (C(T) Values>26)

Brain, Foetal Brain, Colon, Foetal Liver, Heart, Kidney, Lung, Placenta, Prostate, Salivary Gland, Skeletal muscle, spleen, Thymus, Trachea, Uterus, Small intestine, Spinal Cord, Stomach, Pancreas

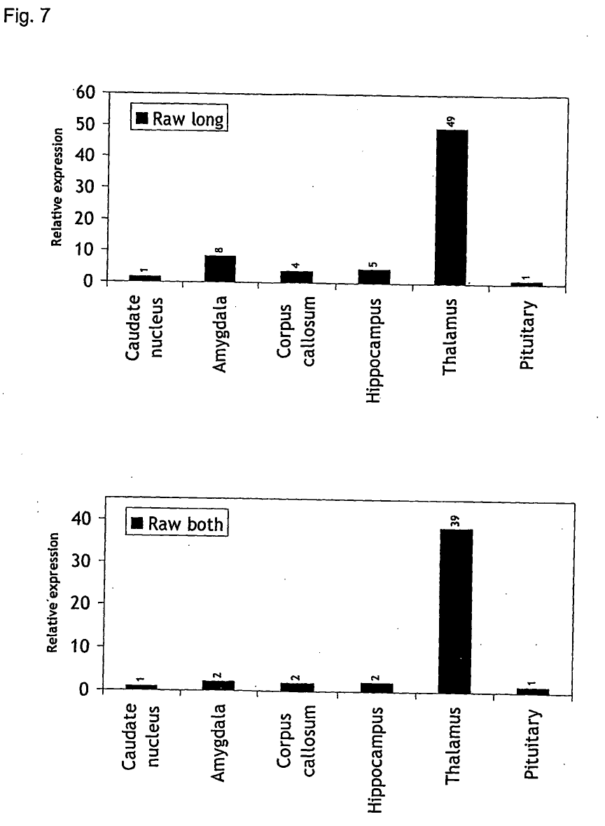

### Analysis of Poly(A)RNA (FIGS. 7-8)

### High Expression (C(T) Values<26)

### Thalamus

### Low Expression (C(T) Values≧226)

Amygdala, Caudate nucleus, Corpus Callosum, Hippocampus, Pituitary

Conclusions drawn from data obtained from analyses performed with primer set NsG1129/1130 detecting the long form. The same conclusions were drawn from data obtained with another primerset (NsG1131/1132) detecting both forms. The tissues, for which no relative expression data are shown in the lower panels of FIGS. 4-5, have not been tested in the PCR which detects both the long and short form of human NsG31.

### Example 4b

### NsG29 Quantitative Expression Data

### Method

Primers amplifying cDNA fragments of 100-350 bp were designed using CloneManager software. Total RNAs derived from foetal and adult human tissues were purchased from Clontech, Dnase treated to remove residual chromosomal DNA and used as templates for cDNA synthesis using an RnaseH deficient reverse transcriptase. cDNA equivalent to 21 ng total RNA was used for each PCR reaction which were carried out using a DNA engine 2 Opticon light cycler from MJ research.

Real-time PCR was performed in an Opticon-2 thermocycler (MJ Research), using LightCycler-FastStart DNA Master SYBR Green I kit (Roche). Studies were carried out in duplicates using primers 5′ oligo: 5′-CCTTCTGCTTCTACCAGATG-3′, annealing at bp 1371 in GenBank sequence: AL713702 and 3′ oligo: 5′-GCAGTCTAGGACAGCTATAC-3′, annealing at bp 1692 in GenBank sequence: AL713702 amplifying a 332 bp fragment in the last exon of the NsG29 cDNA in GenBank sequence: AL713702. For Real-Time PCR, a standard curve was prepared by serial dilution of a gel-purified PCR product, prepared using the above primers. The standard curve was used to verify that crossing-point values (C(T)) of all samples were within the exponential range of the PCR reaction and to calculate final expression levels. All RT-PCR amplifications were performed in a total volume of 10 μl containing 3 mM MgCl₂, 12% sucrose and 1× reaction buffer included in the LightCycler kit. PCR cycling profile consisted of 95° C., 10′ [symbol] 35 cycles: 95° C., 10″ [symbol] 62° C., 20″ [symbol] 72° C., 20″ [symbol] plate read 72° C., 2′. The specificity of the amplification reaction was determined by performing a melting curve analysis of the PCR fragments by slowly raising the temperature from 55° C. to 95° C. with continuous data acquisition.

For normalization purposes, all cDNAs were subjected to real-time PCR using primers for P2-microglobulin (B2M, 5′-TGTGCTCGCGCTACTCTCTC-3′ and 5′-CTGAATGCTCCACTTTTTCAATTCT-3′). Standard curves for P2-microglobulin were prepared similar to NsG29. β₂-microglobulin gene real-time PCR was done using the same kit as for the target gene, except a different annealing temperature was used.

β₂-microglobulin expression levels were determined from the standard curve and the relative expression levels were used to normalize expression levels of the target genes in the tissues that were analyzed. Following normalization, relative expression levels of the target gene were calculated using the tissue with the lowest expression as a reference. Normalized data in FIG. 5 should be interpreted with caution as β₂-microglobulin levels vary between some tissues.

Data are shown in FIGS. 19 and 20.

### Analysis of Total RNA Samples

### High to Moderate Expression (C(T) Values<26)

### Brain, Substantia Nigra, Dorsal Root Ganglion, Putamen

### Low Expression (30≧C(T) Values≧26)

Spinal Cord, Retina, testis, cerebellum, foetal liver

### Very Low or No Expression (C(T) Values>30)

Heart, pancreas, muscle

### Analysis of Poly(A)RNA

### High Expression (C(T) Values<21)

### Hippocampus, Amygdala

### Intermediate (26≧C(T) Values≧21)

### Thalamus, Corpus Callosum, Caudate Nucleus,

### Very Low or No Expression (C(T) Values>26)

### Pituitary

### NsG29, Gene-Chip Experiments

The human material comes from discarded tissue pieces obtained from electively terminated pregnancies using the regular vacuum aspiration technique. The collection of residual tissue for the study is approved by the Human Ethics Committee of the Huddinge University. Hospital, Karolinska Institute (Diary Nr. 259/00) and Lund University (970401), and is in accordance with the guidelines of the Swedish National Board of Health and Welfare (Socialstyrelsen), including an informed consent from the pregnant women seeking abortions. Recovered nervous tissue is micro-dissected within 2 hours of surgery and appropriate tissue fragments are further dissociated for cell isolation.

### RNA Isolation:

Human foetal tissue (8 weeks) was obtained in two rounds, both 8 weeks gestation age. Dissected VM and DM regions were used for total RNA isolation with good results and yields.

Total RNA was isolated with the Trizol extraction following the manufacturer's instruction (Invitrogen) from ventral and dorsal mesencephalic regions subdissected from human foetal tissue, 8 weeks gestational age. To concentrate RNA and to remove traces of chromosomal DNA, Rneasy columns combined with the RNase-Free DNase Set are used following the manufacturer's instructions.

From 5 μg of total RNA, biotinylated cRNA is prepared and fragmented as described in Affymetrix protocols (GeneChip Expression Analysis, Technical Manual 2000) and hybridized (15 μg) to Affymetrix Human U133B GeneChips (containing approximately 22,000 genes) according to manufacturers instructions. Scanned images are analyzed and converted to expression index values using the GenePublisher analysis software package (Knudsen S, Workman C, Sicheritz-Ponten T, Friis C. (2003) “GenePublisher: Automated analysis of DNA microarray data.”, Nucleic Acids Res. 31(13):3471-6).

In the Table below, probe signals obtained by hybridizing 15 μg biotinylated cRNA to Affymetrix Human U133B Gene Chips are shown.

| NsG | Probe-id | Mes1 | Mes2 | Mes3 | Mes4 | Mes5 | Mes6 |
|---|---|---|---|---|---|---|---|
| 29 | 230923 at | 167 | 177 | 137 | 174 | 153 | 159 |

Probe regions for NsG29 with the id's given in the Table were located in the 3′UTRs. The probe signals indicated significant expression of NsG29 in the developing human midbrain.

### Example 5

### Testing for General Neuroprotective Effect (PC12 Assay)

### Generation of Virus Stock:

NsG29 and/or NsG31 and/or NsG31-long coding sequences are subcloned into pHsCXW using appropriate restriction sites as described in Example 3a and 3b. To generate virus stocks, the resulting lentiviral transfer vector is cotransfected into 293T cells with two helper plasmids (PMD.G and pBR8.91) providing the necessary viral genes, gag-pol and env, respectively, in trans. Briefly, 2×10⁸ 293T cells are seeded in each of 20 T75 culture flasks. The next day, each T75 flask is transfected with 15 μg ppBR8.91, 5 μg pMD.G and 20 μg of transfer vector using Lipofectamine+following the manufacturers instructions (invitrogen). Virus-containing medium is harvested 2-3 days after the transfection and filter-sterilized through a 0.45 μm cellulose acetate or polysulphonic filter. The virus is pelleted by double ultracentrifugation at 50,000×g for 90 minutes at 4° C. and then resuspended in DMEM medium. Virus is titrated using a reverse transcription (RT) assay (Current Protocols in Molecular Biology, Editors: Ausubel et al., Willey). The number of transducing units (TU)/ml is calculated from the resulting RT activity and frequency of fluorescent cells obtained by transduction of 293T cells with an equivalent GFP lentivirus. The virus stock is stored in aliquots at −80° C. until use.

### Transduction of PC12 Cells:

PC12 cells are cultured in Dulbecco's modified Eagle's medium (DMEM) with 4.5 g/l glucose and glutamax (Life Technologies #32430-027) with 7.5% donor horse serum (Life Technologies #16050-098) and 7.5% FBS (Life Technologies # 10099-141) in the presence of 5% CO₂ at 37° C. Medium is changed every 2-3 days and cells are subcultured 1:3-1:6 twice a week by tapping the flask and dispensing into new flasks. The day before transduction, cells are seeded in 48-well or 6-well plates coated with collagen. Virus is added from the stock solution to 1 ml cell culture medium together with or without 5 μg/ml (final conc.) polybrene. The virus is incubated with the cells for 24 hours in a CO₂ incubator. A lentiviral GFP vector is added to a parallel culture to estimate transduction efficiency and to serve as control.

### Effect on PC12 Differentiation:

Cultures in 6-well plates are followed and scored for the number of neurite bearing cells after 2-5 days.

### Effect on PC12 Survival

After transduction of cells in 48-well plates, medium is changed to serum-free DMEM and cell viability is measured after 3-4 days using the MTS assay following the manufacturer's instructions (Promega).

A positive effect in either the neurite outgrowth and/or the survival assay is indicative of a potential therapeutic effect of the encoded secreted protein in treating neurodegenerative disorders.

### Example 6

### Protection of Cerebellar Granule Cells from Glutamate Toxicity

Testing for survival effects is carried out by transducing cultures of cerebellar granule cells that subsequently is exposed to toxic concentrations of glutamate essentially as described (Daniels and Brown, 2001; J. Biol. Chem. 276: 22446-22452).

Cerebellar granule neurons (CGN) are dissected from 78 days old mouse pups. Cells are dissociated from freshly dissected cerebella by enzymatic disruption in the presence of trypsin and DNase and then plated in poly-D-lysine-precoated 24-well plates (Nunc) at a density of 1-2×10⁶ cells/cm² in DMEM medium supplemented with 10% heat-inactivated foetal calf serum. Cells are cultured at 37° C. in a humidified atmosphere and Cytosine arabinoside (10 μM) is added to the culture medium after 24 hr to arrest the growth of non-neuronal cells.

Cultures are transduced with an NsG29 and/or NsG31 containing lenti-virus prepared as described in Example 5 on DIV1 by the addition of virus stock solution to DMEM medium containing 10% Foetal bovine serum and 4 μg/ml Polybrene. Parallel control cultures are transduced with a Green Fluorescent Protein (GFP) lentivirus. Five hours after the transduction, medium is replaced with medium preconditioned on CGNs.

At DIV5, glutamate (0.1-1 mM) is added the culture and after two additional days cell survival is assayed using the MTT assay. The extent of MTT reduction to formazane is measured spectrophotometrically at 570 nm. Briefly, culture medium is removed, and cells are washed in sodium saline solution (140 mM NaCl, 5 mM KCl, 1 mM MgCl₂. 6H₂O, 1 mM NaH₂PO₄, 1.5 mM CaCl₂, 5.6 mM glucose, 20 mM HEPES, pH 7.4). MTT (final concentration 0.5 mg/ml), prepared just before using and maintained in the dark in sodium saline solution, is then added to the cells. After a 3 h incubation at 37° C., an equal volume of acid-isopropanol (0.04 M HCl in isopropanol) is added and mixed thoroughly at room temperature until all formazan crystals were dissolved. Cell viability is expressed as a percentage of the optical densitly of control cells. Parallel cultures are left untreated.

This assay can be considered as a general assay for testing of protection against excitotoxic damage as well as an assay predictive for factors with therapeutic potential in the treatment of cerebellar disorders.

### Example 7

### Protection of Cerebellar Granule Cells from Apoptosis Induced by Potassium Deprivation

Testing for survival effects is carried out by transducing cerebellar granule cells deprived of potassium essentially as described (Nomura et al., 2001; Dev. Neurosci. 23: 145-152).

Cerebellar granule neurons (CGN) are dissected from 8-d-old Sprague-Dawley rat pups. Cells are dissociated from freshly dissected cerebella by enzymatic disruption in the presence of trypsin and DNase and then plated in poly-L-lysine-precoated 96-well plates (Nunc) at a density of 3.5×10⁵ cells/cm² in Eagle's basal medium containing 25 mM KCl and supplemented with 10% heat-inactivated foetal calf serum, 2 mM glutamine. Cells are cultured at 37° C. in a humidified atmosphere and Cytosine arabinoside (10 μM) is added to the culture medium after 24 hr to arrest the growth of non-neuronal cells.

Cultures are transduced with an NsG29 and/or NsG31 containing lenti-virus prepared as described in Example 5 [“Testing in PC12 cells”] on DIV1 by the addition of virus stock solution to DMEM medium containing 10% Foetal bovine serum and 4 μg/ml Polybrene. Parallel control cultures are transduced with a GFP lentivirus. Five hours after the transduction, medium is replaced with medium preconditioned on CGNs.

At DIV2, apoptosis is induced in immature cultures by switching the cells to serum-free medium containing 5 mM KCl, while the untreated cells received conditioned medium containing 25 mM KCl. Survival is measured on DIV3, using the MTS assay.

At DIV8, apoptosis is induced in differentiated (neuronal) cultures by switching the cells to serum-free medium containing 5 mM KCl, while the untreated cells received conditioned medium containing 25 mM KCl. Survival is measured after 24-72 hr, using the MTS assay.

The MTS assay is carried out using the The CellTiter 96® AQᵤₑₒᵤₛ Non-Radioactive Cell Proliferation Assay (Promega) following the manufacturer's instructions.

This assay can be considered as a general assay for neuroprotective effects as well as an assay predictive for factors with therapeutic potential in the treatment of cerebellar disorders.

### Example 8

### Effect on DRG cultures

Preparation of conditioned media from transduced ARPE-19 cells. To transduce ARPE-19 cells with a lentivirus containing cDNA encoding the NsG29 and/or NsG31 gene, cells are plated at a density of 1×10⁵ cells/well in a 6-well plate in DMEM/F12 medium supplemented with 10% Foetal Bovine Serum. Next day virus is added from the stock solution to the cell culture medium together with 5 μg/ml (final conc.) polybrene. The virus is incubated with the cells overnight in a CO₂ incubator. GFP lentivirus is added to a parallel culture. The next day, cultures are changed to serum-free UltraCULTURE medium (1 ml/well) and conditioned media are harvested after two additional days of incubation.

Isolation and culture of P1 DRG cells. DRGs from all spinal levels are removed from P1 (post-natal day 1) Sprague-Dawley. Tissues are enzymatically dissociated in 125-250 U/ml type 1 collagenase (Worthington, Freehold, N.J.) at 37° C. for 30 minutes. Samples are triturated with fire-polished Pasteur pipettes and filtered though 70 μm sterile mesh to produce single cell suspensions. Cells are pre-plated on non-coated tissue-culture-ware dishes for 2 hours to remove non-neuronal cells. Non adherent cells are plated at 15,000 cells/well in 24-well tissue culture dishes that had been coated with poly-d-ornithine (Life Technologies) and laminin (Collaborative Biomedical). Negative controls are cultured in UltraCULTURE™ serum-free media, (BioWhittaker, Walkersville, Md.) containing 2.5 μg/ml sheep-neutralizing anti-NGF pAb (Chemicon, Temecula, Calif.). NGF-treated positive controls lacked the neutralizing anti-NGF pAb. Different dilutions of conditioned medium collected from NsG29 and/or NsG31-transduced or GFP-transduced ARPE-19 cells are added to the cultures after centrifugation and filtering through a 0.4 μm sterilfilter. Cultures are fed every second day by replacing the media.

Immunocytochemistry. After seven days in culture, cells are fixed in 4% formaldehyde in PBS for 10 minutes at room temperature. Cells are pre-blocked in 4% goat serum, 0.1% NP40 for 30 minutes at room temperature and then incubated with mouse anti-βIII tubulin (1:100) overnight at 4° C. After rinsing in pre-block solution, the cultures are incubated with a secondary Cy-3 coupled anti-murine antibody for 1 hour at room temperature. Following a final rinse in pre-block solution, cells from a strip through the middle of each well are counted using fluorescence optics. All βIII-tubulin positive cells are scored as neurons and survival is determined by the number of neurons counted per well. All antibodies are diluted in pre-block solution.

Interpretation of results: Protective effects in this assay indicates therapeutic potential in peripheral neuropathies and neuropathic pain.

### Example 9

### Effect on Motoneuron Cultures

Testing for survival effects on motoneuron cultures is carried out using NsG29 and/or NsG31 containing lentivirus essentially as described in Cisterni et al. 200 (J. Neurochem. 74, 1820-1828). Briefly, ventral spinal cords of embryonic day 14.5 (E14.5) Sprague Dawley rat embryos are dissected and dissociated. Motoneurons are purified using a protocol based on the immunoaffinity purification of motoneurons with antibodies against the extracellular domain of the neurotrophin receptor, p75, followed by cell sorting using magnetic microbeads (Arce et al. 1999). Purified motoneurons are seeded on 4-well tissue culture dishes precoated with polyornithine/laminin at density of 500 cells per well. Culture medium is Neurobasal culture medium (Life Technologies) supplemented with the B27 supplement (Life Technologies), horse serum (2% v/v), L-glutamine (0.5 mM), and 2-mercaptoethanol (25 μM). L-Glutamate (25 μM) is added to the medium during the first 4 d of culture and subsequently omitted.

Motoneurons cultured for 16 h are transduced with an NsG29 and/or NsG31 containing lenti-virus prepared as described above by the addition of virus stock solution to the culture medium (corresponding to MOI=4). Parallel control cultures are transduced with a GFP lentivirus. Eight hours after the transduction, medium is replaced with fresh medium (DIV1).

Motoneuron survival is quantified at DIV3 by counting the number of large phase-bright neurons with long axonal processes in a predetermined area of 1.5 cm² in the center of duplicate dishes.

Interpretation of results Protective effects in this assay indicates therapeutic potential in motoneuron diseases including ALS, Spinal Cord injury, SMA (spinal muscular atrophy), DMD (Duchenne muscular dystrophy).

### Example 10

### Bioassay for Dopaminergic Neurotrophic Activities

### Culture Conditions:

Dissociated mesencephalic cell cultures are prepared as previously described (Friedman and Mytilineou 1987 Neurosci. Lett. 79:65-72), with minor modifications. Briefly, rostral mesencephalic tegmentum from brains of Sprague-Dawley rat embryos, at the 13^{th}-16^{th} day of gestation, are dissected under the microscope in sterile conditions, collected in Ca²⁺- and Mg²⁺-free Dulbecco's phosphate buffered saline (Gibco, Gaithersburg, Md.) and dissociated mechanically by mild trituration. The cells are plated in 100 μl per well onto 16-mm diameter tissue culture wells (Falcon, Lincoln Park, N.J., 24-well plate) containing 400 μl medium to give a density of 2.5-3.5×10⁵ cells per well. The culture wells have been previously exposed to 0.1 mg/ml solution of poly L-ornithine in 10 mM sodium borate, pH 8.4, for 3 hours at 37° C., washed 3 times in milli-Q H₂O and once in Earle's balanced salt solution (Gibco). The feeding medium (10/10) consists of minimal essential medium (MEM, Gibco) supplemented with glucose (33 mM), sodium bicarbonate (24.5 mM), glutamine (2 mM), HEPES (15 mM), penicillin G (5 U/ml), streptomycin (5 μg/ml), 10% heat-inactivated foetal calf serum (Gibco) and 10% heat inactivated horse serum (Gibco). The cultures are kept at 37° C. in a water-saturated atmosphere containing 6.5% CO₂. After 3 hours, when most of the cells have adhered to the bottom of the well, the medium is replaced with 500 μl of fresh medium. At this time, a serial dilution of the sample to be assayed for dopaminergic neurotrophic activity (conditioned medium) is added to each well in duplicate and the plates are incubated in the 37° C. incubator. After a week, the cultures are treated for 24 hours with fluorodeoxyuridine (13 μg/ml) and uridine (33 μg/ml) to prevent excessive glial proliferation and subsequently fed with the above medium without foetal calf serum. The feeding medium is changed weekly.

Alternatively, chemically defined serum-free medium is used in which serum is replaced by a mixture of proteins, hormones and salts. The defined medium (DM) consists of a mixture of MEM and F12 nutrient mixture (both Gibco, 1:1; vol/vol) with glucose (33 mM), glutamine (2 mM) NaHCO₃ (24.5 mM), HEPES (15 mM), supplemented with transferrin (100 μg/ml), insulin (25 μg/ml), putrescine (60 μM), progesterone (20 nM), sodium selenite (30 nM), penicillin G (5 U/ml) and streptomycin (5 μg/ml). The osmolarity of the DM is adjusted to 325 by the addition of milli-Q H₂O. (110-125 ml H₂O/I).

The functional status of the dopaminergic neurons may be assayed in these cultures by measuring dopamine uptake through specific “scavenger” transporters in the dopaminergic neurons and by counting the number of neurons positive for the dopamine synthetic enzyme tyrosine hydroxylase using immunohistochemistry as described in Karlsson et al, 2002, Brain Res. 2002 Nov. 15; 955(1-2):268-80.

### Sample Preparation:

Prior to being assayed for dopaminergic neurotrophic activity in the mesencephalic cell cultures, all the samples of conditioned medium are desalted as follows. One hundred μl of the medium 10/10 (as a carrier) is added to a Centricon-10 (Amicon) and allowed to sit for 10 minutes. Aliquots of the sample to be assayed are added to the Centricon, followed by 1 ml of Dulbecco's high glucose Modified Eagle medium, without bicarbonate, but containing 10 mM HEPES, pH 7.2 (solution A), and centrifuged at 5,000×g for 70 minutes. The retentate (about 0.1 ml) is brought back to 1.1 ml with fresh solution A and reconcentrated twice. The sample is filtered through a 0.11 μm Ultrafree-MC sterile Durapore unit (Millipore, Bedford Mass.) prior to being added to the culture well.

### ³H-Dopamine Uptake:

Uptake of tritiated dopamine (³H-DA) is performed in cultures at day 6 or day 7 as described previously (Friedman and Mytilineou (1987) Neurosci. Lett. 79:65-72) with minor modifications, and all the solutions are maintained at 37° C. Briefly, the culture medium is removed, rinsed twice with 0.25 ml of the uptake buffer which consists of Krebs-Ringer's phosphate buffer, pH 7.4, containing 5.6 mM glucose, 1.3 mM EDTA, 0.1 mM ascorbic acid and 0.5 mM pargyline, an inhibitor of monoamine oxidase. The cultures are incubated with 0.25 ml of 50 nM ³H-DA (New England Nuclear, Boston, Mass. sp. act 36-37 Ci/mmol) for 15 minutes at 37° C. ³H-DA uptake is stopped by removing the incubation mixture and cells are then washed twice with 0.5 ml of the uptake buffer. In order to release ³H-DA from the cells, the cultures are incubated with 0.5 ml of 95% ethanol for 30 min at 37° C., and then added to 10 ml of EcoLite (ICN, Irvine, Calif.) and counted on a scintillation counter. Blank values are obtained by adding to the uptake buffer 0.5 mM GBR-12909 (RBI), a specific inhibitor of the high-affinity uptake pump of the dopamine neurons (Heikkila et al. 1984 Euro J. Pharmacol. 103:241-48).

The number of TH positive neurons can be quantified by staining for TH as described previously by Grasbon-Frodl and Brundin, Experimental brain research, 1997 113:138-143 and quantified according to the method described by Karlsson et al, Brain Research, 1998, 805:155-168.

An increase in the number of TH positive neurons and/or an increase in 3H-dopamine uptake compared to a control treatment is an indication of a possible function of NsG29 and/or NsG31 in the treatment of Parkinson's disease.

The mesencephalic cultures described here also comprise motoneurons. By measuring the increase in ChAT (Choline acetyltransferase), an indication of the effect of NsG29 and/or NsG31 on the cholinergic system can be obtained. An activation of ChAT over background is an indication of potential therapeutic use in the treatment of human motoneruron diseases such as ALS (Zurn et al, Neuroreport, 1994, 30:113-118).

### Example 11

### Assessment of Neuroprotection of Nigral Dopamine Neurons In Vivo in the Instrastriatal 6-OHDA Lesion Model

VSV-G pseudotyped (rLV) vectors are produced as described in Example 5. All work involving experimental animals are conducted according to the guidelines set by the Ethical Committee for Use of Laboratory Animals at Lund University. Animals are housed in 12:12 hour light/dark cycle with access to rat chow and water. Female Sprague Dawley rats (˜2209 by the time of surgery) are used. For stereotaxic surgery animals are anesthetized using halothane and a total of two microliters rLV-GFP (n=8) or rLV-NsG29 and/or LV-NsG31 of a 1:2 viral stock (1.0-1.2×10⁵ TU) are Injected into two tracts in the right striatum at the following coordinates: (1) AP=+1.0 mm, ML=−2.6 mm, DV=−5.0 and −4.5 mm, Tb=0.0 and (2) AP=0.0 mm, ML=−3.7 mm, DV=−5.0 and −4.5 mm, Tb=0.0. After two weeks the animals are again anesthetized and placed in the stereotaxic frame. An injection of 6-hydroxydopamine (20 μg [calculated as free base] per 3 μl vehicle [saline with 0.2% ascorbic acid]) is made into the right striatum at the following coordinates: AP=+0.5 mm, ML=−3.4 mm, DV=−5.0 and −4.5 mm, Tb=0.0.

At four weeks post-lesion the animals are deeply anesthetized with pentobarbital (70 mg/kg, Apoteksbolaget, Sweden), and transcardially perfused with 50 ml saline at room temperature, followed by 200 ml ice-cold phosphate-buffered 4% paraformaldehyde (pH 7.2-7.4). The brains are postfixed for 3-6 hours in the same fixative, transferred to 30% sucrose for 24 hours and cut into 6 series of 40 μm thick sections on a freezing microtome.

Immunohistochemistry for detection of tyrosine hydroxylase-immunoreactive, in the substantia nigra is performed as described previously (Rosenblad et al., Molecular and Cellular Neuroscience, 2000, 15:199-214). The number of TH-IR and VMAT-IR nigral neurons is assessed by counting under microscope all immunoreactive neurons lateral to the medial terminal nucleus of the accessory optic tract in three consecutive sections through the SN, as described previously (Sauer & Oertel, Neuroscience 1994, 59:401-415).

An increase in the number of TH-IR compared to the GFP control is a strong indication of a function in the treatment of Parkinson's disease. An increase in the number of VMAT-IR further strengthens the conclusion.

### Example 12

### Effect on Neuronal Differentiation of Human Neural Progenitor Cells

To test for effect on neuronal differentiation, human neural progenitor cells were plated on different substrates after lenti-viral transduction.

Establishment of cultures from first trimester human forebrain tissue has been described in the literature (Carpenter et al., 1999 Exp. Neurol. 158, 265-278). Cells are growing as floating spheres in N2 medium supplemented with 20 ng/ml human EGF (R&D Systems), 20 ng/ml human bFGF (R&D Systems), 10 ng/ml human CNTF (R&D Systems) and 2 μg/ml heparin. In some cases EGF are omitted from the growth medium. N2 medium consists of DMEM: F12 (1:1) (Life Technologies) supplemented with 0.6% glucose, 2 mM glutamine, 5 mM HEPES and N2 supplement (containing insulin, transferrin, progesterone, putrescine and selenium chloride available from Life Technologies).

Cultures are transduced with an NsG29 and/or NsG31 and/or NsG31-long containing lenti-virus prepared as described in Example 5. Furthermore, parallel transduction with a control virus containing EGFP are carried out. Briefly, cultures are triturated to a single cell suspension three days before transduction. On the day of transduction, the small spheres of cells are collected by centrifugation and resuspended in growth medium supplemented with 10% human serum albumin (HSA) at a density of 1×10⁶ cells per ml. Virus is added (MOI=1-5) and cultures are incubated for 4 h. After transduction, cells are collected by centrifugation, resuspended in N2 medium without growth factors and plated on 12-mm glass coverslips coated with poly-L-lysine (PLL, from Sigma, 100 μg/ml) and laminin (LN, from Sigma, 50 μg/ml). Approximately 100,000 cells are plated per coverslip.

After 1-4 days after plating, cells are fixed in 4% paraformaldehyde in PBS for 20 min at room temperature. The cells are then washed three times with PBS, followed by overnight incubation with primary antibodies diluted in PBS incubation buffer which contained 10% normal goat serum, 0.3% Triton X-100 and 1% BSA at 4° C. in a humidified chamber. The cells are washed with PBS, and incubated for 1 h at room temperature in the dark with secondary antibodies diluted in incubation buffer. After washing with PBS, nuclei are counterstained with Hoechst 33342. Negative controls (omission of the primary antibody) are included in each experiment.

The primary antibodies used are; mouse anti-β-tubulin (Sigma 1:400), rabbit anti-GFAP (DAKO 1:100), mouse-antiGalC (Chemicon), and rabbit anti-tyrosine hydroxylase (TH, Chemicon). The secondary antibodies used in these experiments are: anti-mouse FITC (Sigma 1:128) and anti-rabbit Cy3 (Chemicon 1:500).

### Interpretation of Results

After 1-4 days of differentiation, on PLL/laminin substrate control cells transduced with lenti-EGFP have migrated out from the spheres and differentiated to a mixture of neurons (β-tubulin positive cells) and astrocytes (GFAP positive cells). Only few or no TH- or GalC-positive cells are seen.

n increase in the neuron/astrocyte ratio indicates that NsG29 and/or NsG31 and/or NsG31-long has an effect on neuronal differentiation and/or survival.

The presence of GalC positive cells after differentiation indicates that NsG29 and/or NsG31-long and/or NsG31 has an effect on oligodendrocyte specification/differentiation.

The presence of TH positive cells after differentiation indicates that NsG29 and/or NsG31-long and/or NsG31 has an effect on dopaminergic specification/differentiation.

### Example 13

### Real-Time PCR on Cys10 Family Mouse Orthologues

### Materials & Methods:

Primers: The following primers were used for real-
time PCR:
mNsG29:
mNsG29 bp28 intronspan 5′:
5′-AACTGTGGMACGGGACTGG-3′
mNsG29 bp341 intronspan 3′:
5′-TGATGCTGCTGGAAGGTGTG-3′
mNsG31:
mNsG31S + L bp469 intronsp 5′:
5′-AGAAGTGGTGGTGTCAGATG-3′
mNsG31S + L bp626 intronsp 3′:
5′-TCAAGGCTGTCCTACCTAAG-3′
GAPDH:
mGAPDH-s904:
5′-AACAGCAACTCCCACTCTTC-3′
mGAPDH-as1067:
5′-TGGTCCAGGGTTTCTTACTC-3′
mALDH1A1:
mALDH1A1_590fwd,
5′-AAACTCCTCTCACGGCTCTTC-3′
mALDH1A1_859rev,
5′-CAATGTCCAAGTCGGCATCTG-3′
mOTX2:
mOTX2_269fwd,
5′-CCGCCTTACGCAGTCAATGG-3′
mOTX2_611rev,
5′-TCACTTCCCGAGCTGGAGAG-3′
mGDNF:
mGDNF_95s
TATGGGATGTCGTGGCTGTC
mGDNF_341as,
GCTGCCGCTTGTTTATCTGG

### Preparation of Expression Panel:

Tissue from different brain regions of E10.5, E11.5, E13.5, P1 and adult mice was Isolated and RNA prepared by Trizol extraction. Subsequent on-column DNAse treatment using RNeasy spin columns was done to remove traces of gDNA and to further clean the RNA. Aliquots of 2.5 μg RNA was used as template for cDNA synthesis with an RNAseH deficient reverse transcriptase derived from MOMLV (SuperScript) and poly-dT pimer. cDNA from all samples were synthesised at the same time using the same mastermix to avoid variations. The final volume of the cDNA reaction was 120 μl, which was stored in aliquots at −80° C. to avoid repeated thawing and freezing. The expression panel consists of cDNA prepared from the following tissues; dorsal forebrain (DFB), ventral forebrain (VFB), ventral mesencephalon (VM), dorsal mesencephalon (DM) and spinal cord (SC) from 10.5 and 11.5 days old embryos. In addition, cortex (CTX), medial and lateral ganglionic eminences (MGE/LGE), DM, VM and SC from 13.5 days old embryos were included. Furthermore, from newborn mouse (P1), cerebellum (Cb), CTX, VM, DM, and MGE/LGE were used and finally Cb, CTX, VM, DM, and SC were used from adult mouse.

### Real Time PCR Expression Analysis:

For real-time PCR expression analysis, approximately 20 ng of each cDNA was used as template. Real-time PCR was performed in an Opticon-2 thermocycler (MJ Research), using LightCycler-FastStart DNA Master SYBR Green I kit (Roche). Studies were carried out in duplicates using the primers described above. For real-time PCR, a standard curve was prepared by serial dilution of a gel-purified PCR product, prepared using the above primers. The standard curve was used to verify that crossing-point values (CT) of all samples were within the exponential range of the PCR reaction and to calculate final expression levels. All real-time PCR amplifications were performed in a total volume of 10 μl containing 3 mM MgCl₂, 12% sucrose and 1× reaction buffer included in the LightCycler kit. PCR cycling profile consisted of a 10 minutes pre-denaturation step at 98° C. and 35 three-step cycles at 98° C. for 10 seconds, at 62° C. (mGAPDH), 65° C. (mALDH1A1), 65° C. (mOTX2), 60° C. (mGDNF), 62° C. (mNsG28-32) for 20 seconds and at 72° C. for 20 seconds. Following the extension step of each cycle, a plate reading step was added (80° C., 2 seconds) to quantify the newly formed PCR products. The specificity of the amplification reaction was determined by performing a melting curve analysis of the PCR fragments by slowly raising the temperature from 52° C. to 95° C. with continuous data acquisition.

For normalization purposes, all cDNAs were subjected to real-time PCR using primers for the housekeeping gene GAPDH. Real-time PCR analysis of GAPDH was done as for the target genes. Housekeeping expression pattern was determined from the respective standard curves and the relative expression levels were used to normalize expression levels of the target genes in the tissues that were analysed. Following normalization with GAPDH, relative expression levels of the target genes were calculated using the tissue with the lowest expression as a reference.

### Results:

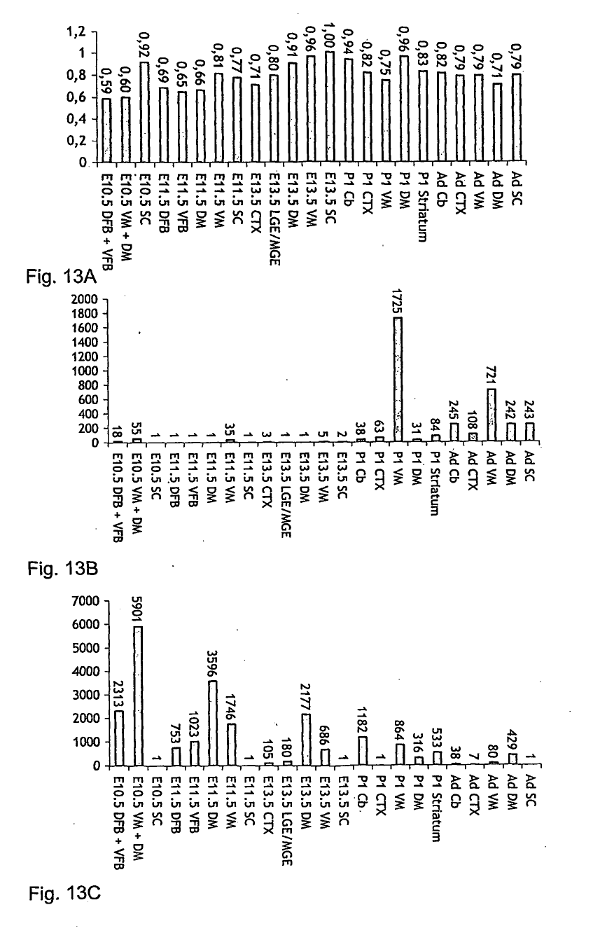

To verify tissue dissections and subsequent RNA isolation and cDNA preparation, the expression profile of the marker genes OTX2 and ALDH1A were investigated. OTX2 is expressed in the forebrain and primarily in the dorsal part of the midbrain with a posterior boundary at the isthmic organiser. The retinoid synthesizing enzyme ALDH1A1, is a specific marker of developing dopaminergic neurons in the ventral midbrain. Hence, the expression profile of these two genes can be used to validate the cDNA panel. It is apparent from FIG. 13 that the expression level of the housekeeping gene GAPDH differs less that 50% between tissues. In contrast, very differentiated expression profiles are observed for ALDH1A1 and OTX2. As expected, in the fetal tissues and at P1, ALDH1A1 is expressed almost exclusively in the ventral midbrain. Also as expected, during development, OTX2 is expressed in the forebrain and (dorsal) midbrain but not in the spinal cord. Together, this is evidence of a high quality expression panel of the developing mouse central nervous system (CNS).

Several proteins have been described that have been shown to have therapeutic properties relevant to the central nervous system. Common to these molecules are conservation across species, features of growth factors, and expression during development in specific regions of the nervous system. The regional and temporal expressions are also predictive for the therapeutic indications in a majority of cases. For example, GDNF is one growth factor that is known to be therapeutically relevant for the nervous system. This molecule is conserved across species and, during development, GDNF is expressed in the ventral mesencephalon and the striatum at the time of terminal differentiation of the nigro-striatal dopaminergic system (FIG. 14). GDNF is in fact a therapeutic molecule for Parkinson's Disease and its therapeutic properties have been demonstrated in several animal models of PD.

The real-time PCR results for mouse NsG31 are shown in FIG. 15. The CT values ranged from 30 to 35, i.e. an overall low expression level compared to the expression levels estimated for human tissue (example 4). This could be caused by the primer-set. From this figure it is apparent that the expression peaks in P1 VM (from which the substantia nigra develops) and P1 LGE/MGE (from which the striatum develops). This regional and temporal expression pattern indicates a role in the differentiation and termination of the projections between the VM and the striatum. A therapeutic effect on neurons involved in Huntingtons' and Parkinson's disease is thus indicated.

The real-time PCR results for mouse NsG29 are shown in FIG. 23. CT values for NsG29 ranged from 20 to 31. From this figure it is apparent that the expression peaks in P1 VM (from which the substantia nigra develops) and P1 LGE/MGE (from which the striatum develops). mNSG29 additionally displays strong expression in cerebellum at P1.

NsG29 is also a secreted protein conserved across species with features of a growth factor or hormone. Its regional and temporal expression pattern during development indicate a peak in expression in various areas of the nervous system during the late foetal and neonatal period that are involved in terminal differentiation. The pattern of expression indicates a peaking in the caudal areas of the CNS, e.g. spinal cord before expression in the rostral regions. This coincides with the myelination of the nervous system. The retention of partial expression in the adult CNS may indicate a role in the myelination and maintenance of the axon sheaths and oligodendrocytes. Expression in the adult amygdale and hippocampus may indicate a role in stem cell biology and neurogenesis as these regions show extensive plasticity and stem cell proliferation during adulthood.

### Example 14

### Promotion of Rod Photoreceptor Survival in Organotypic Cultures from Mice with Inherited Retinal Degeneration (rd1 Mouse)

Retinitis Pigmentosa (RP) comprises one of the most genetically heterogeneous groups of inherited disorders. Mutations in nearly 30 distinct genes have thus far been associated with progressive blindness both in animals and humans (http://www.sph.uth.tmc.edu/Retnet/home.htm).

Defects in the gene encoding the β subunit of rod cGMP-phosphodiesterase (β-PDE) have been identified among RP patients and accounts for 3 to 4% of recessive RP (McLaughlin M E, Ehrhart T L, Berson E L, Dryja T P (1995) Mutation spectrum of the gene encoding the beta subunit of rod phosphodiesterase among patients with autosomal recessive retinitis pigmentosa. Proc Natl Acad Sci U S A 92:3249-53). Retinal degeneration 1 (Pde6b^{rd1}) found in the rd1 mouse is caused by two mutations, a nonsense mutation together with an insertion of a retrovirus in the β-PDE gene (Bowes, C., Li, T., Danciger, M., Baxter, L. C., Applebury, M. L., and Farber D. B. (1990) Retinal degeneration in the rd mouse is caused by a defect in the beta subunit of rod cGMP-phosphodiesterase. Nature 347, 677-680; Bowes C, Li T, Frankel W N, Danciger M, Coffin J M, Applebury M L, Farber D B (1993) Localization of a retroviral element within the rd gene coding for the beta subunit of cGMP phosphodiesterase. Proc Nat Acad Sci (USA) 90:2955-9; Pittler S J, Bähr W (1991) Identification of a nonsense mutation in the rod photoreceptor cGMP phosphodiesterase beta-subunit gene of the rd mouse. Proc Nat Acad Sci (USA) 88:8322-6). Both in humans and in the mouse, defects in β-PDE result in a non-functional β-PDE protein (an enzyme localized in the photoreceptor outer segments and involved in the phototransduction processes). This, in turn, results in chronically open cGMP-gated cation channels and consequent overload of the rod cells with Ca²⁺, leading to rapid degeneration. In mice homozygous for the mutation, the retinal rod photoreceptor cells begin degenerating at about postnatal days (P) 7-8. Cell loss is detectable at around P11, and by P20, nearly all rod photoreceptor cells (98%) have been lost (Carter-Dawson L D, LaVail M M, Sidman R L (1978) Differential effect of the rd mutation on rods and cones in the mouse retina. Invest Opthalmol V is Sci 17:489-498; Rohrer B, Pinto F R, Hulse K E, Lohr H R, Zhang L, Almeida J S (2004) Multidestructive pathways triggered in photoreceptor cell death of the rd mouse as determined through gene expression profiling. J Biol Chem 279:41903-10).

The rd1 mouse is extensively used as a model for human RP in studies aimed at elucidating the mechanisms of cell death and for the screening of substances with neuroprotective properties. The early onset and the rapid progression of the degeneration in the rd1 mouse makes it also suitable for in vitro studies. A culture system has been developed in which a retinal explant can be kept under serum-free and long-term (up to 4 weeks) conditions (see below). As the rod photoreceptor cells of rd1 mice degenerate also in culture, the effects of protective substances or combinations of substances can be tested by controlled addition of such molecules to the culture medium. Organotypic cultures of rd1 mouse retinas are typically established from 5-day-old mice (P5) and maintained for 15-25 days (P20-30), thus covering the period of maximal photoreceptor cell death.

The following example refers to test of NsG31. NsG31-long and NsG29 may be tested using similar methods.

### Tissue Culture Preparation

The preparation of organotypic retinal cultures has been previously described (CafféAR, Ahuja P, Holmqvist B, Azadi S, Forsell J, Hoimqvist I, Söderpalm AK, van Veen T (2001a) Mouse retina explants after long-term culture in serum free medium. J Chem Neuroanat 22:263-73). Briefly, eyes from homozygous rd1 mice (C3H) and congenic wild-type mice (P5) are quickly enucleated under sterile conditions and placed in dishes containing R16 serum-free basal medium (R16-SFBM; Invitrogen Life Technologies, Paisley, UK) (Romijn H J (1988) Development and advantages of serum-free, chemically defined nutrient media for culturing of nerve tissue. Biol Cell 63:263-8; Romijn H J, de Jong B M, Ruljter J M (1988) A procedure for culturing rat neocortex explants in a serum-free nutrient medium. J Neurosci Methods 23:75-83.) supplemented with 0.12% proteinase K (MP Biomedicals, LLC, Eschwege, Germany) at 37° C. for 15 min. Eyes are then transferred to R16 basal medium containing 10% Fetal Calf Serum (FCS, Invitrogen, Stockholm, Sweden) for 2 min at room temperature and subsequently to R16-SFBM.

Eyes are hemisected (˜0.5 mm behind the limbus) under R16-SFBM and the anterior segment, lens and vitreous body discarded. Retinas are carefully peeled off leaving the neural retina intact with the retinal pigment epithelium (RPE) attached. Four radial cuts are made perpendicular to the edges of each retina-RPE, allowing the explants to be mounted flat with the photoreceptor side down onto Millicell® Culture Plate Inserts (Millipore, Sweden). The inserts are placed on 6-well culture dishes, each containing 1.5 mL of conditioned medium from 293T cells transfected with NsG31 or MOCK transfected (see description below), diluted 1:10 in R16 serum-free complete medium (R16-SFCM) (Caffé AR, Ahuja P, Holmqvist B, Azadi S, Forsell J, Holmqvist I, Söderpalm AK, van Veen T (2001a) Mouse retina explants after long-term culture in serum free medium. J Chem Neuroanat 22:263-73). Retina-RPE explants are cultured for 15-25 days at 37° C. (5% CO₂; 100% humidity), with changes of medium every second day. In another series of experiments, 10 ng/mL recombinant human brain derived neurotrophic factor (BDNF, PeproTech EC Ltd, London, UK) is added to the NsG31/MOCK medium. BDNF has previously been shown to act synergistically with other growth factors (ciliary neurotrophic factor, CNTF) in this culture system (Caffé AR, Söderpalm AK, Holmqvist I, van Veen T (2001b) A combination of CNTF and BDNF rescues rd photoreceptors but changes rod differentiation in the presence of RPE in retinal explants. Invest Opthalmol V is Sci 42:275-82).

The assay may also be run using purified NsG31 protein (with or without tag). In that case one or more concentrations of NsG31 protein is dissolved in the R16 serum-free medium and the assay proceeds as otherwise described. NsG31 is preferably tested at a concentration corresponding to BDNF and at least one concentration above and at least one above.

### Conditioned NsG31 Medium

293T cells were seeded in T75 flasks (2×10⁶ cells per flask). The next day, cells were transfected with a plasmid containing cDNA for human NsG31, using LipofectAMINE PLUS according to the manufacturer's instructions. In parallel, cells in a T75 flask were transfected with a C-terminally His-tagged version of human NsG31 and a His-tagged version of GDNF. After 24 hrs, medium was changed to pre-heated HE-SFM (10 mL per T75 flask) and incubated for 24 hrs. Medium was then harvested and centrifuged at 500 rpm for 10 min to spin cells and debris down. Conditioned medium from human NsG31 and MOCK transfected cells was frozen down in aliquots for testing in retinal cultures. Samples from cells transfected with cDNA for human NsG31 and GDNF-His was analysed by His-Tag WB analysis to estimate the concentration of growth factors in the medium. The concentration of human NsG31 was estimated to be 60-70 nM. Cells transfected with human NsG31, human NsG31-His and MOCK-transfected cells were washed in PBS and harvested in Trizol and processed for RNA isolation and cDNA synthesis. RT-PCR analysis using primers for human NsG31 was performed to ensure expression of human NsG31 mRNA.

### Tissue Processing

At the end of the culturing period, the medium is removed from the culture dish and replaced with 4% paraformaldehyde (PFA) in 0.1 M phosphate buffer (PBS, pH 7.4). After 2 hours, the fixative is removed and replaced with Sörensen's phosphate buffer (2×15 min), and thereafter with 0.1 M Sörensen's phosphate buffer (pH 7.3) containing increasing concentrations of sucrose (5/10/25% sucrose; ≧5 hrs each). Explants are embedded, cryosectioned, and stored at −20° C. until processed.

### Determining Photoreceptor Survival

Cryostat sections (16 μm each) are obtained from entire NsG31-treated and control explants and stained with hematoxylin-eosin. In a first analysis, the number of rows of photoreceptor cell nuclei in the outer nuclear layer is determined and used as a measure of photoreceptor survival. The number of rows is counted in every 9^{th} section (i.e., at 144 μm intervals) in 5 points (at 500 μm intervals) in each section. This analysis yields 65-75 points of measurement per retinal explant, covering an area of approximately 2400 μm². Specimens exhibiting poor structure or pyknotic cells in any of the retinal layers are excluded from the analysis. The data obtained from each group is analyzed using one-way ANOVA at 5% significance level, followed by Fisher's protected least significant difference post-hoc comparisons. A difference between groups is regarded as significant at *p<0.05; **p<0.01; ***p<0.0001. Damaged DNA is also detected in retina-RPE explants cultured for 15 days by the indirect TUNEL (Terminal deoxynucleotidyl Transferase Biotin-dUTP Nick End Labelling) method, using the ApopTag® Fluorescein Direct In Situ Apoptosis Detection Kit (Chemicon International Inc. Temecula, Calif., USA), according to the manufacturer's instructions.

In addition, some sections are processed by immunocytochemistry using an antibody against rhodopsin, in order to assess whether the surviving photoreceptors express the specific photoreceptor cell marker. The cryostat sections are pre-incubated for 90 min with 0.1 M PBS containing 1% BSA, 0.25% Triton X-100 (PBTX), and 5% normal serum. This is followed by overnight incubation at 4° C. with mouse monoclonal anti-rhodopsin (Rho-1D4; 1:400; gift from R. S. Molday, Canada), diluted in PBTX containing 2% normal serum. After rinsing, sections are incubated for 90 min with Texas Red Sulfonyl Chloride conjugated to donkey anti-mouse (1:100; Jackson immunoResearch, USA). After completing the staining procedure, sections are rinsed, coverslipped with buffered glycerol containing the anti-fading, phenylenediamine, and viewed using a light microscope equipped for fluorescence microscopy.

An increased survival rate will indicate that NsG31 may be useful for the treatment of inherited retinal degenerations, such as Retinitis Pigmentosa.

1-99. (canceled)

100. An isolated polypeptide for medical use, said polypeptide comprising an amino acid sequence selected from the group consisting of:
a) the amino acid sequence selected from the group consisting of SEQ ID No. 3, 4, 5, 6, 9, 10, 11, 12, 15, 16, 18, 19, 20, 21, and 23;
b) a sequence variant of the amino acid sequence selected from the group consisting of SEQ ID No. 3, 4, 5, 6, 9, 10, 11, 12, 15, 16, 18, 19, 20, 21, and 23 wherein the variant has at least 95% sequence identity to said SEQ ID No.; and
c) a biologically active fragment of at least 50 contiguous amino acids of any of a) through b).

101. The polypeptide of claim 100, having at least 95% sequence identity to the protein having the sequence of SEQ ID No. 4.

102. The polypeptide of claim 100, having at least 95% sequence identity to the protein having the sequence of SEQ ID No. 16.

103. The polypeptide of claim 100, having at least 95% sequence identity to the protein having the sequence of SEQ ID No. 19.

104. The polypeptide of claim 100, wherein the fragment is selected from the group consisting of:
i) SEQ ID No 5, and polypeptides having from one to five additional amino acids from the mature polypeptide sequence in the C- and/or N-terminal, up to AA₁₀-AA₉₅ of SEQ ID No 4; and
ii) variants of said polypeptides, wherein any amino acid specified in the chosen sequence is changed to a different amino acid, provided that no more than 10 of the amino acid residues in the sequence are so changed.

105. The polypeptide of claim 100, wherein the fragment is selected from the group consisting of:
i) SEQ ID No. 6, and polypeptides having from one to five additional amino acids from the mature polypeptide sequence in the C- and/or N-terminal, up to AA₈-AA₁₀₃ of SEQ ID No 4; and
ii) variants of said polypeptides, wherein any amino acid specified in the chosen sequence is changed to a different amino acid, provided that no more than 10 of the amino acid residues in the sequence are so changed.

106. The polypeptide of claim 100, wherein the fragment consists of AA₁₄-AA₁₃₉ of SEQ ID No. 16 or AA₁₆-AA₁₃₉ of SEQ ID No. 16 or variants of said fragments, wherein any amino acid specified in the chosen sequence is changed to a different amino acids, provided that no more than 15 of the amino acid residues in the sequence are so changed.

107. The polypeptide of claim 100, wherein the fragment is selected from the group consisting of:
i) SEQ ID No. 20, and polypeptides having from one to five additional amino acids from the mature polypeptide sequence in the C- and/or N-terminal, up to AA₁₄-AA₉₈ of SEQ ID No 19; and
ii) variants of said polypeptides, wherein any amino acid specified in the chosen sequence is changed to a different amino acid, provided that no more than 10 of the amino acid residues in the sequence are so changed.

108. The polypeptide of claim 100, selected from the group consisting of:
i) SEQ ID No. 21, and polypeptides having from one to five additional amino acids from the mature polypeptide sequence in the C- and/or N-terminal, up to AA₁₂-AA₁₀₈ of SEQ ID No. 19; and
ii) variants of said polypeptides, wherein any amino acid specified in the chosen sequence is changed to a different amino acid, provided that no more than 10 of the amino acid residues in the sequence are so changed.

109. The polypeptide of claim 100, wherein the changed amino acids are selected from those designated as unconserved, weakly conserved or strongly conserved in FIG. 4 or 18.

110. The polypeptide of claim 100, wherein the changed amino acids are selected from those designated as unconserved, weakly conserved or strongly conserved in FIG. 1B, more preferably in FIG. 1A.

111. The polypeptide of claim 100, wherein any changed amino acid residue is changed to a residue found at the same or corresponding position in another Cys10 protein (FIG. 1A or 1B).

112. The polypeptide of claim 100, comprising the ten conserved cysteine residues of the Cys10 family at positions corresponding to the position of mature NsG29 or NsG31.

113. The polypeptide of claim 100, comprising the following sequence:
G-T-C-E-[V/I]-[V/I]-x(3)-R-x(5)-[R/K]-x(5)-Q-T-
[V/A]-[K/R]-C-x-C-x(2)-G-x-[V/I]-A-G-T-T-R-x(2)-P-
x-C-V-[D/E]-A-x-I-[V/I]-x(2)-[K/R]-x-W-C-x-M-x-P-
C-L-x-G-E-x-C-x(2)-L-x(4)-G-W-x-C-x(2-3)-G-x-
[K/R]-[V/I]-K-T-T.

114. The polypeptide of claim 113, comprising the following sequence:
G-T-C-E-V-[V/I]-A-x-H-R-C-C-N-[K/R]-N-[R/K]-I-E-E-
R-S-Q-T-V-K-C-S-C-x(2)-G-x-V-A-G-T-T-R-x(2)-P-S-C-
V-[D/E]-A-x-I-V-x(2)-[K/R]-W-W-C-x-M-x-P-C-L-x-G-
E-[E/D]-C-K-x-L-P-D-x(2)-G-W-x-C-x-[S/T]-G-x-K-
[V/I]-K-T-T-[R/K].

115. The polypeptide of claim 100, being capable of forming at least one intramolecular cystine bridge.

116. The polypeptide according to claim 100, further comprising an affinity tag, such as a polyhis tag, a GST tag, a HA tag, a Flag tag, a C-myc tag, a HSV tag, a V5 tag, a maltose binding protein tag, a cellulose binding domain tag.

117. An isolated nucleic acid molecule comprising a nucleotide sequence selected from the group consisting of:
a) a nucleotide sequence coding for a polypeptide having the amino acid sequence selected from the group consisting of SEQ ID No. 3, 4, 5, 6, 9, 10, 11, 12, 15, 16, 18, 19, 20, 21, and 23;
b) a nucleotide sequence coding for a sequence variant of the amino acid sequence selected from the group consisting of SEQ ID No. 3, 4, 5, 6, 9, 10, 11, 12, 15, 16, 18, 19, 20, 21, and 23 wherein the variant has at least 95% sequence identity to said SEQ ID No.;
c) a nucleotide sequence coding for a biologically active fragment of at least 50 contiguous amino acids of any of a) through b);
d) a nucleotide sequence selected from the group consisting of SEQ ID No. 2, 8, 14, 17 and 22;
e) a nucleotide sequence having at least 95% sequence identity to a coding sequence selected from the group consisting of the coding sequence of SEQ ID No. 2, 8, 14, 17, and 22;
f) a nucleic acid sequence of at least 150 contiguous nucleotides of a coding sequence selected from the group consisting of the coding sequence of SEQ ID No. 2, 8, 14, 17 and 22;
g) the complement of a nucleic acid capable of hybridising with a nucleic acid molecule having the sequence of the coding sequence of SEQ ID No.: 2, 8, 14, 17 and 22 under conditions of high stringency; and
h) the nucleic acid sequence of the complement of any of the above.

118. The nucleic acid molecule of claim 117, wherein the nucleic acid molecule comprises the nucleotide sequence of a naturally occurring allelic nucleic acid variant.

119. The nucleic acid molecule of claim 117, wherein the encoded polypeptide has at least 95% sequence identity to the protein having the sequence of SEQ ID No. 4.

120. The nucleic acid molecule of claim 117, wherein the encoded polypeptide has at least 95% sequence identity to the protein having the sequence of SEQ ID No. 16.

121. The nucleic acid molecule of claim 117, wherein the encoded polypeptide has at least 95% sequence identity to the protein having the sequence of SEQ ID No. 19.

122. The nucleic acid molecule of claim 117, having at least 95% sequence identity to the nucleic acid molecule having the sequence of the coding sequence of SEQ ID No. 2.

123. The nucleic acid molecule of claim 117, having at least 95% sequence identity to the nucleic acid molecule having the sequence of the coding sequence of SEQ ID No. 14.

124. The nucleic acid molecule of claim 117, having at least 95% sequence identity to the nucleic acid molecule having the sequence of SEQ ID No. 17.

125. The nucleic acid molecule of claim 117, having the nucleotide sequence of nucleotides 160-468 of SEQ ID No. 2.

126. The nucleic acid molecule of claim 117, having the nucleotide sequence of nucleotides 160-576 of SEQ ID No. 14.

127. The nucleic acid molecule of claim 117, having the nucleotide sequence of nucleotides 217-618 of SEQ ID No. 17 (human NsG29 CDS).

128. The nucleic acid molecule of claim 117, being codon optimised for expression in E. coli, Chinese Hamster, Baby Hamster, Yeast, insect and/or fungus.

129. A vector comprising the nucleic acid molecule of claim 117.

130. The vector of claim 129, further comprising a promoter operably linked to the nucleic acid molecule.

131. An isolated host cell transfected or transduced with the vector of claim 129.

132. The host cell of claim 131, being attached to a matrix.

133. A packaging cell line capable of producing an infective virus particle, said virus particle comprising a Retroviridae derived genome comprising a 5′ retroviral LTR, a tRNA binding site, a packaging signal, a promoter operably linked to a polynucleotide sequence encoding the polypeptide of claim 100, an origin of second strand DNA synthesis, and a 3′ retroviral LTR.

134. An implantable biocompatible cell device, the device comprising:
i) a semipermeable membrane permitting the diffusion of a protein as defined by claim 100 and/or a virus vector; and
ii) an host cell transfected or transduced with the vector comprising a polynucleotide sequence encoding the polypeptide of claim 100.

135. An implantable biocompatible cell device, the device comprising:
i) a semipermeable membrane permitting the diffusion of a protein as defined by claim 100 and/or a virus vector; and
ii) a packaging cell line capable of producing an infective virus particle, said virus particle comprising a Retroviridae derived genome comprising a 5′ retroviral LTR, a tRNA binding site, a packaging signal, a promoter operably linked to a polynucleotide sequence encoding the polypeptide of claim 100, an origin of second strand DNA synthesis, and a 3′ retroviral LTR.

136. The device of claim 135, wherein the semipermeable membrane is immunoisolatory.

137. The device of claim 135, wherein the semipermeable membrane is microporous.

138. The device of claim 135, wherein the device further comprises a matrix disposed within the semipermeable membrane.

139. The device of claim 135, wherein the device further comprises a tether anchor.

140. The device of claim 135, wherein said device comprises a core comprising living packaging cells that secrete a viral vector for infection of a target cell, wherein the viral vector is a retrovirus, the vector comprising a heterologous gene encoding a polypeptide according to claim 100, operably linked to a promoter that regulates the expression of said polypeptide in the target cell; and an external jacket surrounding said core, said jacket comprising a permeable biocompatible material, said material having a porosity selected to permit passage of retroviral vectors of approximately 100 nm diameter thereacross, permitting release of said viral vector from said capsule.

141. The device of claim 140, wherein the core additionally comprises a matrix, the packaging cells being immobilized by the matrix.

142. The device of claim 140, wherein the jacket comprises a hydrogel or thermoplastic material.

143. A pharmaceutical composition comprising the polypeptide of claim 100 and a pharmaceutically acceptable carrier.

144. A pharmaceutical composition comprising the isolated nucleic acid sequence of claim 117 and a pharmaceutically acceptable carrier.

145. A pharmaceutical composition comprising the vector of claim 129 and a pharmaceutically acceptable carrier.

146. A pharmaceutical composition comprising a composition of host cells according to any of the claims 131 and a pharmaceutically acceptable carrier.

147. A pharmaceutical composition comprising a packaging cell line according to claim 133 and a pharmaceutically acceptable carrier.

148. A pharmaceutical composition comprising an implantable biocompatible cell device according to any of the claims 134 and a pharmaceutically acceptable carrier.

149. A pharmaceutical composition comprising an implantable biocompatible cell device according to any of the claims 135 and a pharmaceutically acceptable carrier.

150. A method of treatment of a pathological condition in a subject comprising administering to an individual in need thereof a therapeutically effective amount of a polypeptide of claim 100.

151. The method of claim 150, wherein said medicament is for the treatment of a disease, disorder, or damage associated with the nervous system.

152. The method of claim 151, wherein said disease, disorder, or damage is characterised by neuronal apoptosis.

153. The method of claim 151, wherein said medicament is for the treatment of a disease, disorder, or damage involving injury to the brain, brain stem, the spinal cord, and/or peripheral nerves, including but not limited to conditions such as stroke, traumatic brain injury, spinal cord injury, diffuse axonal injury, epilepsy, neuropathy, peripheral neuropathy and associated pain and other symptoms.

154. The method of claim 153, wherein the disease is thalamic pain.

155. The method of claim 151, wherein the Nervous System disorder involves degeneration of neurons and their processes in the brain, brain stem, the spinal cord, and/or the peripheral nerves, including but not limited to Parkinson's Disease, Alzheimer's Disease, senile dementia, Huntington's Disease, amyotrophic lateral sclerosis, neuronal injury associated with multiple sclerosis, and associated symptoms.

156. The method of claim 155, wherein the neurodegenerative disease is Parkinson' Disease.

157. The method of claim 155, wherein the neurodegenerative disease is Huntington's Disease.

158. The method of claim 155, wherein the neurodegenerative disease is Alzheimer's Disease.

159. The method of claim 155, wherein the neurodegenerative disease is amyotrophic lateral sclerosis.

160. The method of claim 151, wherein the nervous system disorder is a disease, disorder, or damage involving dysfunction and/or loss of neurons in the brain, brain stem, the spinal cord, and/or peripheral nerves, including but not limited to conditions caused by metabolic diseases, nutritional deficiency, toxic injury, malignancy, and/or genetic or idiopathic conditions including but not limited to diabetes, renal dysfunction, alcoholism, chemotherapy, chemical agents, drug abuse, vitamin deficiency, and infection.

161. The method of claim 158, wherein the disease is essential tremor.

162. The method of claim 158, wherein the disease is peripheral neuropathy and associated pain.

163. The method of claim 151, wherein the nervous system disorder is a disease, disorder, or damage associated with the Cerebellum, including but not limited to sensory ataxia, multiple sclerosis, neurodegenerative spinocerebellar disorders, hereditary ataxia, cerebellar atrophies (such as olivopentocerebellar atrophy (OPCA), Shy-Drager Syndrome (multiple systems atrophy)), and alcoholism.

164. The method of claim 151, wherein the nervous system disorder is a disease, disorder, or damage involving degeneration or sclerosis of glia such as oligodendrocytes, astrocytes and Schwann cells in the brain, brain stem, the spinal cord, and the peripheral nerves, including but not limited to multiple sclerosis, optic neuritis, cerebral sclerosis, post-infectious encephalomyelitis, and epilepsy and associated symptoms.

165. The method of claim 164, wherein the disorder is multiple sclerosis.

166. The method of claim 151, wherein the nervous system disorder, disease, or damage involves the retina, photoreceptors, and associated nerves including but not limited to retinitis pigmentosa, macular degeneration, glaucoma, diabetic retinopathy, and associated symptoms.

167. The method of claim 151, wherein the nervous system disorder, disease, or damage involves the sensory epithelium and associated ganglia of the vestibuloacoustic complex including but not limited to noise-induced hearing loss, deafness, tinnitus, otitis, labyrintitis, hereditary and cochleovestibular atrophies, Menieres Disease, and associated symptoms.

168. The method of claim 150, wherein the subject is a human being.

169. The method of claim 150, wherein the pathological condition is a disease related to testis, including male sterility, impotence, erectile dysfunction, cancer, and germ cell tumours.

170. A method of treatment of a pathological condition in a subject comprising administering to an individual in need thereof a therapeutically effective amount of the isolated nucleic acid sequence of claim 117.

171. A method of treatment of a pathological condition in a subject comprising administering to an individual in need thereof a therapeutically effective amount of the expression vector of claim 129.

172. A method of treatment of a pathological condition in a subject comprising administering to an individual in need thereof a therapeutically effective amount of a composition of host cells according to claim 131.

173. A method of treatment of a pathological condition in a subject comprising administering to an individual in need thereof a therapeutically effective amount of a packaging cell line according to claim 133.

174. A method of treatment of a pathological condition in a subject comprising administering to an individual in need thereof a therapeutically effective amount of an implantable biocompatible cell device according to claim 134.

175. A method of treatment of a pathological condition in a subject comprising administering to an individual in need thereof a therapeutically effective amount of an implantable biocompatible cell device according to claim 135.

176. A method of male contraception comprising administering to a male subject a polypeptide of any of claim 100.

177. A method of male contraception comprising administering to a male subject an isolated nucleic acid sequence of claim 117.

178. A method of male contraception comprising administering to a male subject the expression vector of claim 129.

179. A method of male contraception comprising administering to a male subject a composition of host cells according to claim 131.

180. A method of male contraception comprising administering to a male subject an implantable biocompatible cell device according to claim 134.

181. A method of male contraception comprising administering to a male subject an implantable biocompatible cell device according to claim 135.

182. A method of expanding a composition of mammalian cells comprising administering to said composition a polypeptide of claim 100.

183. A method of expanding a composition of mammalian cells comprising transducing/transfecting the cells with the expression vector of any of the claims 129.

184. A method of differentiating a composition of mammalian cells comprising administering to said composition a polypeptide of claim 100.

185. A method of differentiating a composition of mammalian cells comprising transducing/transfecting the cells with the expression vector of any of the claims 129.

186. An antibody capable of binding to a polypeptide of claim 100.

187. The antibody of claim 186, being selected from the group consisting of: polyclonal antibodies, monoclonal antibodies, humanised antibodies, single chain antibodies, recombinant antibodies.

188. An immunoconjugate comprising the antibody of claim 186 and a conjugate selected from the group consisting of: a cytotoxic agent such as a chemotherapeutic agent, a toxin, or a radioactive isotope; a member of a specific binding pair, such as avidin or streptavidin or an antigen; an enzyme capable of producing a detectable product.

189. An isolated polypeptide having an amino acid sequence selected from the group consisting of SEQ ID No. 5 and 11 and variants of said polypeptides, wherein any amino acid specified in the chosen sequence is changed to a different amino acid, provided that no more than 15 of the amino acid residues in the sequence are so changed.

190. The isolated polypeptide of claim 189, further containing up to 5 additional amino acids in the C- or N-terminal, the additional amino acids preferably being selected from the amino acids at corresponding positions in mature NsG31 (SEQ ID No. 4 and 10).

191. An isolated polypeptide having an amino acid sequence selected from the group consisting of SEQ ID No 6 and 12, and variants of said polypeptides, wherein any amino acid specified in the chosen sequence is changed to a different amino acid, provided that no more than 15 of the amino acid residues in the sequence are so changed.

192. The isolated polypeptide of claim 191, further containing up to 5 additional amino acids in the C- or N-terminal, the additional amino acids preferably being selected from the amino acids at corresponding positions in mature NsG31 (SEQ ID No. 4 and 10).

193. An isolated polypeptide having an amino acid sequence of AA₁₄-AA₁₃₉ of SEQ ID No. 16, and variants of said polypeptide, wherein any amino acid specified in the chosen sequence is changed to a different amino acid, provided that no more than 15 of the amino acid residues in the sequence are so changed.

194. An isolated polypeptide having an amino acid sequence of SEQ ID No. 20, and variants of said polypeptide, wherein any amino acid specified in the chosen sequence is changed to a different amino acid, provided that no more than 15 of the amino acid residues in the sequence are so changed.

195. The isolated polypeptide of claim 189, further containing up to 5 additional amino acids in the C- or N-terminal, the additional amino acids preferably being selected from the amino acids at corresponding positions in mature NsG29.

196. An isolated polypeptide having an amino acid sequence of SEQ ID No 21, and variants of said polypeptide, wherein any amino acid specified in the chosen sequence is changed to a different amino acid, provided that no more than 15 of the amino acid residues in the sequence are so changed.

197. The isolated polypeptide of claim 191, further containing up to 5 additional amino acids in the C- or N-terminal, the additional amino acids preferably being selected from the amino acids at corresponding positions in mature NsG29.

198. An isolated polynucleotide coding for a polypeptide according to claim 189.

199. An isolated polynucleotide coding for a polypeptide according to claim 190.

200. An isolated polynucleotide coding for a polypeptide according to claim 191.

201. An isolated polynucleotide coding for a polypeptide according to claim 192.

202. An isolated polynucleotide coding for a polypeptide according to claim 193.

203. A method of preventing apoptosis in a neuronal cell comprising contacting a neuronal cell with an effective amount of the polypeptide of claim 100.

204. A method of preventing apoptosis in a neuronal cell comprising contacting a neuronal cell with an effective amount the isolated nucleic acid sequence of claim 117.

205. A method of preventing apoptosis in a neuronal cell comprising contacting a neuronal cell with an effective amount of the expression vector of claim 129.
